SEQUENCE LISTING (27 sequences)

SEQ ID NO: 1 - DNA, 1755 bp, Pagrus major
  source 1..1755: mol_type = genomic DNA
atgggagcgctgcgcccgcgctgcctccacctcctcctcctgctgctgggcttctccgtgtccacctcggcggcctgggacctggttctcctccacaccaacgacgtgcacgcccgggtggaggagaccagcaagcactccgggaaatgcagcagcagcaggaagagcggcgggtgtttcgccggagtggcccgcagagccacgatgatcaagaagatccgctccaccgacagcaacgtgctgctgctggacgccggggaccagttccagggcagcgtgtggttcaattactacaagggagccgaggctgctcacttcatgaacaaactgcagtatgatgccatggccttgggaaatcacgagttcgacaacggagtggaaggactcatgaagccgttcatggagaagatcaggtgtcctgttctgagcgccaacatcaaacctgatgaagctatggcaccgacgttcagcagctcctatctaccttataagatactgactgtgggcagcgagaaggtgggcgtagtgggatacacgtcgcaggaaactccggctctgtccagacctggacctcacctggagtttgaggacgaggtgacctcgctgcagctgcaggtgaacaaactgcagacgctgggagtgaataagatcatcgctctgggacactccggcttcacggtggatcgcgagatcgccaagaaggtccgcggagtcgacgtggtcatcggtggacacaccaacactttcctgttcacagggcatcctccctcctctgaggtcccgttgggtagttatccgttcatggtgacatcagtggacgggcgccaggttcctgtcgtgcaggcctacgcctttggaaaatatctgggtcacttgaaagtgacctttgatgatgctggaaacgtgatgaagtcaacaggaaacccaatcctgctggacagcagcgtcccacaggatccagatgttcttgctgacgtggaggaatggaaaaagagtctggccaactactcagctcaggaggtgggaaagactctggtgttcctgaacgggaccactgaggagtgtcgatttcgagaatgcaacctgggaaacctgatctgtgacgccatggtcaacaacaacatccgattcttagaggacgagcagtggaaccacgtcagcgcctccatcttcaatggaggaggcatccgaacatccatcgatgagcacagcaggaacggttcgatcaccatggaggacctgatctccgtcttacctttcggaggaaccttcgacctggtgcagctgaacggctccacgctgaggagagctttcgagcactcagtgaaacgatacggcgaaagcaccggagaatttctccaagtgtccggtttccatgtagagttcgacctctccaaaccggctggtagtcgcgtgaggagcctcgacatcctctgcactcagtgtcgagtccctcaatacgaacccgtggaggatgagacggtttacacagtggtggtgccgtccttcatggtgacaggcggagacggatactccatgatcaggaatgagacgctcaaacacaacagcggtaatctggacatttcagtcgtgtccaactacatcatgcagagaaagagagtttatccggctgttgaaggacgaatcaagatctacaactcagcttctggaccgcgaggacaaattttactggtttcactggtgctgctctggactctgtgggagcatgtaggagtgacttcaaccagtttttaa

SEQ ID NO: 2 - protein, 584 aa, Pagrus major
MGALRPRCLHLLLLLLGFSVSTSAAWDLVLLHTNDVHARVEETSKHSGKCSSSRKSGGCFAGVARRATMIKKIRSTDSNVLLLDAGDQFQGSVWFNYYKGAEAAHFMNKLQYDAMALGNHEFDNGVEGLMKPFMEKIRCPVLSANIKPDEAMAPTFSSSYLPYKILTVGSEKVGVVGYTSQETPALSRPGPHLEFEDEVTSLQLQVNKLQTLGVNKIIALGHSGFTVDREIAKKVRGVDVVIGGHTNTFLFTGHPPSSEVPLGSYPFMVTSVDGRQVPVVQAYAFGKYLGHLKVTFDDAGNVMKSTGNPILLDSSVPQDPDVLADVEEWKKSLANYSAQEVGKTLVFLNGTTEECRFRECNLGNLICDAMVNNNIRFLEDEQWNHVSASIFNGGGIRTSIDEHSRNGSITMEDLISVLPFGGTFDLVQLNGSTLRRAFEHSVKRYGESTGEFLQVSGFHVEFDLSKPAGSRVRSLDILCTQCRVPQYEPVEDETVYTVVVPSFMVTGGDGYSMIRNETLKHNSGNLDISVVSNYIMQRKRVYPAVEGRIKIYNSASGPRGQILLVSLVLLWTLWEHVGVTSTSF

SEQ ID NO: 3 - DNA, 1740 bp, Takifugu rubripes
  source 1..1740: mol_type = genomic DNA
atgagcgtgtggtcgcgccggtgcgctctctgggtctgcatctctctactggcggggccagtttggaccttcgagctgacgctgcttcacaccaacgacaaccatgcgcggatcgaggagaccagcgaggacttgggaaagtgctcatccaggggtccctgtttcgcgggggtcgcccggaggttcaccaaagtgtcggagatccgcaaaaaggaaaagaacgttttgttcctggacgctggggaccaatttcaggggacggtttggtttaactactacaaaggcgcagaagccgcgcactttatgaataaacttggttataatgcgatggctttagggaaccatgagtttgataatggggtggagggactcctgcctttcctccagaatgtgaactgttctgtggtgagtgcgaacatccaacctgaccagagtctggctgcaaagctcagcggtttcctccagccctacacggtcctcaacgtgggctcagagaaggtggccgtggtgggctacaccaccgccgagacccccttcctatctatgccaggcccacatcttaaatttaacgaggaagtggaggcgctccaggtccaggtggacaaactggaaaccctgggttatgataagatcatcgccttgggtcactccggctttgatgtggatcagcagatcgccaagcgagtgagaggggttgacgtcgtcatcggaggacacaccaacaccttcctttatactggaaaagccccgtccactgaagtgcctgtaggtccttatccttttatagtgaggtctgacgatgggagaaacgtcccggtggtccaagcttttgcctttggaaaatacctcggttacttgagggtcacatttgacgatgctggaaaagtaatcaaggctgcaggaaacccaatcctactggacagcagcgtccctcaggatccggacgtcctggctgaggtcaacaggtggaagaaagacctggctcagtactcctcacagtatgtcgggcaaaccttagtttatctcaacggcacgtttgaagagtgccgatttcgggagtgtaacctcggcaacctgatctgcgatggcatgatcgaccacaacatcaaattctccagcgagctgcagtggaaccacgtgagcctttgtatgctgaacagcggagccatacgggctcccatcgatgagcgctacaagaacggctccataactatggaggacgtcctcactgtcttgccgttcggagggactgttgacttggtgcagataaaagggtcaacggtgaagaaggcgttcgaacatgccgtccacagatttggaagcatgtcaggagagtttctgcaagtctcaggcttccatgtcaagtacgacctgtccaaaccagtgaaccaacgcgtcacgtctctgtcggcactttgcactgagtgccgcgtacccaaatatgagcccatagacccggagaggacgtacaaggtgaccatgccgtcatatctggtggacggtggtgacggtttcagcatgatcaaggaggagttactgaagcataacacaggtgacttggacatttctgtgttctctaaatatatctcgcagcagaagcgcgtatatccagcagtggagggccggatcacagtcaggggctcggcctcctctgcagcccacagcctcgacgtattccttcatacttggcctcttctcccatacatttccccaggaagtctgaaagataaatggctatag

SEQ ID NO: 4 - protein, 579 aa, Takifugu rubripes
MSVWSRRCALWVCISLLAGPVWTFELTLLHTNDNHARIEETSEDLGKCSSRGPCFAGVARRFTKVSEIRKKEKNVLFLDAGDQFQGTVWFNYYKGAEAAHFMNKLGYNAMALGNHEFDNGVEGLLPFLQNVNCSVVSANIQPDQSLAAKLSGFLQPYTVLNVGSEKVAVVGYTTAETPFLSMPGPHLKFNEEVEALQVQVDKLETLGYDKIIALGHSGFDVDQQIAKRVRGVDVVIGGHTNTFLYTGKAPSTEVPVGPYPFIVRSDDGRNVPVVQAFAFGKYLGYLRVTFDDAGKVIKAAGNPILLDSSVPQDPDVLAEVNRWKKDLAQYSSQYVGQTLVYLNGTFEECRFRECNLGNLICDGMIDHNIKFSSELQWNHVSLCMLNSGAIRAPIDERYKNGSITMEDVLTVLPFGGTVDLVQIKGSTVKKAFEHAVHRFGSMSGEFLQVSGFHVKYDLSKPVNQRVTSLSALCTECRVPKYEPIDPERTYKVTMPSYLVDGGDGFSMIKEELLKHNTGDLDISVFSKYISQQKRVYPAVEGRITVRGSASSAAHSLDVFLHTWPLLPYISPGSLKDKWL

SEQ ID NO: 5 - DNA, 1755 bp, Oreochromis niloticus
  source 1..1755: mol_type = genomic DNA
atgtgctatacattcttggtcacaaatgcgttttttacgcacggagacatcatgggtttccgttcgtctcggcgcgctctcttgacctcgctctgcctcgttttaaactgctggagcggagcgtcgaccttcgagctgacaatactccacaccaatgacaaccatgcgcgaatcgaggagaccggcaaggactcgggaaagtgtcgacccgagcgtccctgcttcgctggtgtggccaggaggttcacgaaagtgaccgagatcagaacaaaagagacgaacgtggtgtttctggatgctggagaccaattccaagggacactctggttcaactactataaaggcgctgaagccgcacactttatgaacaaactttgttatgatgttatggcttttggaaatcacgagtttgacaatggagtggagggtctcattcaccccttcctccaaaatataaatttctctgtggtcagtgcaaacattaaacccgactacaccctggcagagcttcataaatactacagtccctacaaagttatcagtgtgggctcagagaaagtggctgtggttggctatacctcagcagagacgcccttcttatccatgccaggcaaacatctcaagttcgaagatgaggtggagtcacttcaagctcaagtaaataagctggaaagtctgggctataataaaatcattgccctgggacactctggttttgttgtggatcaagacatcgcaagacgcgtgagaggggtggacgttgttgttggagggcataccaacacgttcctctacacaggaacgcccccatcttccgaagtgccagctggcccatatccttttatagtaaaatccagccatggaagagatgtgccagtggtccaggcttacgcctttgggaaatatcttggacacttaaaagtcacctttgatgatgctggcaatgtcatcaaagccgttggaaatcccatcctaatggacagcagcatacctcaagatgcagagatccttgctgatgttaataagtggaaaacagacttggctcaatactcgactaagtatgtgggccaaaccttagtctacctcaacgggtcatttgaagagtgccgatttcgggagtgtaacctgggaaacctaatctgtgatgccatggtgtatcacaatatgaggcattcaactggggagcagtggaatcatgtaagcctgtgtatgctgaatagtgggggcatacggacagcgatagacgaacgctacagaaatggctccataacaatggaggagatcctcaccgtgttaccatttggaggaacctgcgacttggtccaaataaaaggctcaacaataaaaaaggcatttgaacactctgttcacagatatggaagcaagactggagaatttcttcaggtggtttacgacctctctaagccagtgaaccagcgtgtagcatcgctgtcattgctctgcacagagtgccgagtgcccaagtatgaaccattagacccacagaagacctacaccgtggtcatgccttcatacatggtgggcggaggcgataactttacaatgattaagagggagttactgaaacataattcaggtgacttggacattaccgttttttccaagtacatctccgacatgaagcgcgtgttccctgcagtagaaggccgcatcactttcaggaactctgctgtcattgcttcatacagtattggcttgttgctgctgagtctgtgtctgtctgtgacactttga

SEQ ID NO: 6 - protein, 584 aa, Oreochromis niloticus
MCYTFLVTNAFFTHGDIMGFRSSRRALLTSLCLVLNCWSGASTFELTILHTNDNHARIEETGKDSGKCRPERPCFAGVARRFTKVTEIRTKETNVVFLDAGDQFQGTLWFNYYKGAEAAHFMNKLCYDVMAFGNHEFDNGVEGLIHPFLQNINFSVVSANIKPDYTLAELHKYYSPYKVISVGSEKVAVVGYTSAETPFLSMPGKHLKFEDEVESLQAQVNKLESLGYNKIIALGHSGFVVDQDIARRVRGVDVVVGGHTNTFLYTGTPPSSEVPAGPYPFIVKSSHGRDVPVVQAYAFGKYLGHLKVTFDDAGNVIKAVGNPILMDSSIPQDAEILADVNKWKTDLAQYSTKYVGQTLVYLNGSFEECRFRECNLGNLICDAMVYHNMRHSTGEQWNHVSLCMLNSGGIRTAIDERYRNGSITMEEILTVLPFGGTCDLVQIKGSTIKKAFEHSVHRYGSKTGEFLQVVYDLSKPVNQRVASLSLLCTECRVPKYEPLDPQKTYTVVMPSYMVGGGDNFTMIKRELLKHNSGDLDITVFSKYISDMKRVFPAVEGRITFRNSAVIASYSIGLLLLSLCLSVTL

SEQ ID NO: 7 - DNA, 1635 bp, Oncorhynchus mykiss
  source 1..1635: mol_type = genomic DNA
atgaaaaagggtaaccagcatataatgtatgcgcctaaaattattcccaatgttttggtcgctttcctttccctagcggctgagattctttttttctccaaaagtgcgtcagacacggttactttcacttggttctcacttgccgaggaacccgtgtcaccggagtgggtatcatgtcttccacactgcaaaggtgatgtcgatgccacagatccaggggagttcgtgaccaggcgcgctgaagcatttggaaaccatgagtttgacaatggagtggagggcctgctccgcccttttctgcagaaggtgaacttcactgttctcagtgccaacatcaaggcagatgcaacactcgccccaactatcaatggttactatcaaccctacacaacattcacgatgggctctgaaatagtggctgtggtaggttacacctcagtggaaactccagtcttatccttaccaggtccacacctgatctttgaggatgagatcaaagccttgcaggtccaggtggataaactgatcactcttggttacaataagatcatcgcccttggtcactctggatttgacgtggatatagacatcgccaagcgagtgaaaggcgtggacctggtcatcggcggacacaccaacactttcctctacaccgggagtgtcccatcttcagaggtgccggcgggtccctaccccttcacggtgaggtctgaggacgggagagatgtccctgtggtacaggcttttgcctttgggaagtacctgggatacctcaagttggtcttcgataaatctgggaacgtgctgaaggcgaatggaaaccccatcctgctggacagcagcatagcccaggatccaggtatccttgctgatgttgacgagtggaagaagaacttggcccagtactcgtctcagtacgtggggaaaaccctggtgtatctcaatggaaccttcaatgagtgccggttccgagagtgcaacctggggaacctcatctgtgacgcaatgattcatcacaatatcaagtatgcagatgagatccagtggaatcacgtcagtttatgcatcctgaacagtggaggcataaggacaggaatagatgaaagtcacaaaaatggcaccatcaccatggaggaagttatatcagtcttaccatttgggggcacttttgatctggtgcagttgaagggatcaaccttgaaaaaggcatttgaaaactctgtccgaagatacgggagcagcagaggagaatttcttcaagtttcaggaatccatgtggaatacgacctgtcacggtcggtgggggaccgtgtgacgtccttgagcctccgctgctctcagtgccgtgtgcccagatacgaatctctggacccagacaggctgtacaagctggtcctaccatcctacatcgcagacggaggagatggttttaccatgattaaggaggagaaactgaaacatgacactggtgatctggacatctcggttttcgccaactacatcaaggagatgaagagggtgtacccaactgtggagggccgcatcaagttcagaaactcatccgtggcggctggggccaactgtctgactttgttactgctaggcctgatgtgggccctgtccacaagtttatga

SEQ ID NO: 8 - protein, 544 aa, Oncorhynchus mykiss
MKKGNQHIMYAPKIIPNVLVAFLSLAAEILFFSKSASDTVTFTWFSLAEEPVSPEWVSCLPHCKGDVDATDPGEFVTRRAEAFGNHEFDNGVEGLLRPFLQKVNFTVLSANIKADATLAPTINGYYQPYTTFTMGSEIVAVVGYTSVETPVLSLPGPHLIFEDEIKALQVQVDKLITLGYNKIIALGHSGFDVDIDIAKRVKGVDLVIGGHTNTFLYTGSVPSSEVPAGPYPFTVRSEDGRDVPVVQAFAFGKYLGYLKLVFDKSGNVLKANGNPILLDSSIAQDPGILADVDEWKKNLAQYSSQYVGKTLVYLNGTFNECRFRECNLGNLICDAMIHHNIKYADEIQWNHVSLCILNSGGIRTGIDESHKNGTITMEEVISVLPFGGTFDLVQLKGSTLKKAFENSVRRYGSSRGEFLQVSGIHVEYDLSRSVGDRVTSLSLRCSQCRVPRYESLDPDRLYKLVLPSYIADGGDGFTMIKEEKLKHDTGDLDISVFANYIKEMKRVYPTVEGRIKFRNSSVAAGANCLTLLLLGLMWALSTSL

SEQ ID NO: 9 - DNA, 1713 bp, Gnathopogon caerulescens
  source 1..1713: mol_type = genomic DNA
atgatggcacagtggctcagctccataagcctcctctggattcactgtcagctgtgcaggactgcggagttcgagctgactttactccacacgaatgatgtccacgcacgagtggaggagaccaataaagactcggggaagtgcagtaaagcgccttgtttcgctggagtcgctcggaggttgacgaagatcagggaagtccgagcccaggagaaacacgttctgctgctggacgcgggagaccagttccagggcaccgtctggtttaacttttacaaaggcgcagaagcggcgtatttcatgaataaactcggatacaatgcgatggccttaggaaatcatgagtttgacaatggagtggacggcctcgtgaaacctttccttcaggaagtgaactgctctgttctgagcgcaaatattaaagccgatcagacaatcgctcctcggatcagcggctactatctcccctataaaatcttcaatttcacctcagagaaagtaggtgttgttggctacacatccgtggagaccccggctttgtctctgccaggcccccatctccagtttgaggatgaggttactgctttacagcttcaagtggacaaactcactgctttgggtgttaataagatcattgcccttggacattctggcttcctaacggacaaaaagatcgcaaagaaagttcggggagtggatgttgtgattggagggcacaccaatacattcttgttcacaggagagccaccatccacagaggttcccgcaggaccgtacccattcatggtggattctgaggatggccgacaggttcccgtggtccaggcctatgcatttggaaaatatctgggattcctaaaagtgacttttgattcaaatgggaatgtggtgaaatcttttggcaacccaattcttctgaatggctcagtagcaccagatccagtcattcaggctgaagtggacaactggaggaagaatctggccaattactcttctcagtatgtaggagagactctcgtctatctcaatgggacttttgaggaatgccgatttcgtgaatgtaatttgggaaatttaatctgtgatgcaatggttcataataatattaaatatgctgatgaaatccagtggaaccatgtcagctcttgtattctgaacggtggagccattcgatcacctattgatgagcgaaacagaaatggttccatcacgatggaggacctgatcgctgtcctgccgtttggaggcacgtttgacctggtccaaatgaacggatctactctgcgagaggtctttgagcactcagttcgcagatacggaggaaacactggagaattcctacaggtgtcagggtttcagctggtgtttgatttatcgaaaccgccaggaagccgtgttaaaagtgtgaacgcgctctgcacggagtgtcgagtgccccgctatgaaccgctcatccccacgaaggtgtataaagtggtgctgccctcttacctagtggacggaggagacggatacaccatgatcaaagaacagaaactcaaacatgacagtggtgacctggatatagccgtagttgccagctatatcacagagagaaagagagttcatccagccgtggaaggacgcattcagttttccagctcttgcattgcacatcgaggatacacggccatcgttctgctggtgtgggcgctctgggtcatgattgtttag

SEQ ID NO: 10 - protein, 570 aa, Gnathopogon caerulescens
MMAQWLSSISLLWIHCQLCRTAEFELTLLHTNDVHARVEETNKDSGKCSKAPCFAGVARRLTKIREVRAQEKHVLLLDAGDQFQGTVWFNFYKGAEAAYFMNKLGYNAMALGNHEFDNGVDGLVKPFLQEVNCSVLSANIKADQTIAPRISGYYLPYKIFNFTSEKVGVVGYTSVETPALSLPGPHLQFEDEVTALQLQVDKLTALGVNKIIALGHSGFLTDKKIAKKVRGVDVVIGGHTNTFLFTGEPPSTEVPAGPYPFMVDSEDGRQVPVVQAYAFGKYLGFLKVTFDSNGNVVKSFGNPILLNGSVAPDPVIQAEVDNWRKNLANYSSQYVGETLVYLNGTFEECRFRECNLGNLICDAMVHNNIKYADEIQWNHVSSCILNGGAIRSPIDERNRNGSITMEDLIAVLPFGGTFDLVQMNGSTLREVFEHSVRRYGGNTGEFLQVSGFQLVFDLSKPPGSRVKSVNALCTECRVPRYEPLIPTKVYKVVLPSYLVDGGDGYTMIKEQKLKHDSGDLDIAVVASYITERKRVHPAVEGRIQFSSSCIAHRGYTAIVLLVWALWVMIV

SEQ ID NO: 11 - DNA, 1173 bp, Paralichthys olivaceus
  source 1..1173: mol_type = genomic DNA
tcaggacctcacctgaggttcgaggacgaggtggacgctctgcagccacatgtggacaagctgcggacgctgggagtggataagatcatcgctctgggtcactctggtttcaccaaggaccaggagatcgctaagaaggtccgcggagtggacgtggtcatcggaggacacaccaacacgttcctctacacaggaactcctccctcatcggaagtccccgcaggtccgtatccctttctggtgaaatcggacgacgggcggcaggttcccgttgtgcaggcctacgccttcggaaaatatctgggatatctgaaagtgacctttgacgatgctggaaacgtggagagggccacaggaaaccccatcctgctgaacagcagcttcccgcaggacccagatgttctggctgatgtggagaaatggaagaagaatctggcaaactactcggctcaggttgtgggacagactctggtcttcctgaacggagaaagcgaggagtgtcgtttccgggagtgtaacctgggaaacctgatctgtgacgccatggtcgacaacaacatccgcatccctgacgacgtccagtggaaccacgtcagcgccagcatcttcaacggaggcggcgtccgggcgtccatcgacgagcagtccaggaacggttccatcaccatggaggacctgatctccgtcttaccgttcggaggaaccttcgacctggtgcagctgaggggctccacgctgaggaaagcgttcgaacactcagtgagacgatacggacagagcaccggagaattcctccaagtgtccggatttcacgtagagttcgatctctccaaacctccaggacgtcgtgtgacgagcctccgcatcctctgcacagagtgtcgcgtccctcactaccaaccggtggaggacgagacggtttacacggtggtgctgacgtcctacatggtgaagggcggggacggattcgacatgatccaaaacgagattgtcaagtacaacagcggagatctggacatttcagtcgtttccaggttcatcgggaagagaaagaaagtttatcctcctgtcgaaggacgaatcaggatcaagaactctgcgtcgaggctgcagggacgagctgctctgctggtttcactgtcgctgctctggagtgtgtgcgggaccatgtga

SEQ ID NO: 12 - protein, 390 aa, Paralichthys olivaceus
SGPHLRFEDEVDALQPHVDKLRTLGVDKIIALGHSGFTKDQEIAKKVRGVDVVIGGHTNTFLYTGTPPSSEVPAGPYPFLVKSDDGRQVPVVQAYAFGKYLGYLKVTFDDAGNVERATGNPILLNSSFPQDPDVLADVEKWKKNLANYSAQVVGQTLVFLNGESEECRFRECNLGNLICDAMVDNNIRIPDDVQWNHVSASIFNGGGVRASIDEQSRNGSITMEDLISVLPFGGTFDLVQLRGSTLRKAFEHSVRRYGQSTGEFLQVSGFHVEFDLSKPPGRRVTSLRILCTECRVPHYQPVEDETVYTVVLTSYMVKGGDGFDMIQNEIVKYNSGDLDISVVSRFIGKRKKVYPPVEGRIRIKNSASRLQGRAALLVSLSLLWSVCGTM

SEQ ID NO: 13 - DNA, 1734 bp, Oryzias latipes
  source 1..1734: mol_type = genomic DNA
atgactctccgctggcgctgctgcgccctcggcgccttgctcggtctccttctccggttagactcgtggagcggagcgtccggcttcgagctgactctgctccacaccaacgacgtccacgctcgcatcgaggagaccagcgaggactcgtccaaatgccacgaagcgggctcgtgcttcgcgggggtcgccaggatgttcaccaaagtgacggagatccggagaaaggagacgcacgtgctgtttctggacgctggagatcaatttcaaggcacggtgtggttcaactactacaaaggcaaagaagctgcgcatttcatgaacaaacttggttatgacgtcatgacctttgggaaccacgagtttgataatggagtggacagtctcacgcaaaacttcctccagcgcgtaaacttctccgtagtgtgtgccaccatcaaaccgctccacagtctggttgcaaacatgagccgcttctacagaccctttgctgtttttaacgtgggctcagagaaggttgctgttgttggttacaccaccaaagaaacccctgtcttatctgctccaggtccttatctgaagtttgaggatgaggtggaagccctccaggatcaggtcaaccagctagagaagctgggtgtgaacaagatcatcgcattgggacactctggctttgaagtggacaaagacatcgccaagcgtgtgagaggaatcgatgtagtgatcggaggacacacaaacaccttcctgtacacaggaaagcctccttctaccgaagtgccgagaggaccctatcctttcaatgtgagctccaacgacgggagatgggtgccagtggtccaagcgttcgccttcggaaaatacctgggatacctgaaggtgacgttcgaccaagctgggaaggtcgtaaaagccgttggaaaccccatcctgatgaacagcagcatccctcaagatccagggatcttgtcggatgttgaaaagtggaagaagggcttggagcagtattcttcccagtacattggacaaactttagtctacctgaatggaacgtttgaagagtgtcgatttagggaatgtaacctggggaacctcatctgtgatgccatgatatacaactatattaggtattccaacaagctgcagtggaatcacgtgggtgtttgtatgctgaacagtggatccatacgagcaacaatcgatgaacgcagtacaaacggctccatcacgatggaggagattctcagcgtcttgccgtttggaggaacttttgacttagtgcagctgaaaggatctacgctaaaaaaggcttttgagcactctgttcacagatatggagggatgtctggagaatttcttcaagtgtcaggtatccgtgtggagtacgacctctccaaacccgtcaaccagcgcgttgtatcgctgttaatgcgttgcacagaatgccgcgttcccaagtttgaaccgctggaccctcagaagacgtacaccgtggtcatgacctcattcatggtgggcggaggcgatggctatagcatgatccaagatgaattattgaagcataacacaggaaatttggacactttggttttttccgaatacatcaaggacatgaggcgcgtgtaccctgcagtggaaggccgcatcaccttcaggaactctgcagcttttgcagctcacagcctgagcctgatgctgctgagtctgtgtcttttcctgaacctctgcatgtga

SEQ ID NO: 14 - protein, 577 aa, Oryzias latipes
MTLRWRCCALGALLGLLLRLDSWSGASGFELTLLHTNDVHARIEETSEDSSKCHEAGSCFAGVARMFTKVTEIRRKETHVLFLDAGDQFQGTVWFNYYKGKEAAHFMNKLGYDVMTFGNHEFDNGVDSLTQNFLQRVNFSVVCATIKPLHSLVANMSRFYRPFAVFNVGSEKVAVVGYTTKETPVLSAPGPYLKFEDEVEALQDQVNQLEKLGVNKIIALGHSGFEVDKDIAKRVRGIDVVIGGHTNTFLYTGKPPSTEVPRGPYPFNVSSNDGRWVPVVQAFAFGKYLGYLKVTFDQAGKVVKAVGNPILMNSSIPQDPGILSDVEKWKKGLEQYSSQYIGQTLVYLNGTFEECRFRECNLGNLICDAMIYNYIRYSNKLQWNHVGVCMLNSGSIRATIDERSTNGSITMEEILSVLPFGGTFDLVQLKGSTLKKAFEHSVHRYGGMSGEFLQVSGIRVEYDLSKPVNQRVVSLLMRCTECRVPKFEPLDPQKTYTVVMTSFMVGGGDGYSMIQDELLKHNTGNLDTLVFSEYIKDMRRVYPAVEGRITFRNSAAFAAHSLSLMLLSLCLFLNLCM

SEQ ID NO: 15 - DNA, 336 bp, Oryzias latipes
  source 1..336: mol_type = genomic DNA
atgactctccgctggcgctgctgcgccctcggcgccttgctcggtctccttctccggttagactcgtggagcggagcgtccggcttcgagctgactctgctccacaccaacgacgtccacgctcgcatcgaggagaccagcgaggactcgtccaaatgccacgaagcgggctcgtgcttcgcgggggtcgccaggatgttcaccaaagacggagatccggagaaaggagacgcacgtgctgtttctggacgctggagatcaatttcaaggcacggtgtggttcaactactacaaaggcaaagaagctgcgcatttcatgaacaaacttggttatga

SEQ ID NO: 16 - protein, 111 aa, Oryzias latipes
MTLRWRCCALGALLGLLLRLDSWSGASGFELTLLHTNDVHARIEETSEDSSKCHEAGSCFAGVARMFTKDGDPEKGDARAVSGRWRSISRHGVVQLLQRQRSCAFHEQTWL

SEQ ID NO: 17 - DNA, 60 bp, synthetic construct
  misc_feature 1..60: note = oligoA-sgRNA1
  source 1..60: mol_type = other DNA
ctaatacgactcactatagcaggatgttcaccaaagtgagttttagagctagaaatagca

SEQ ID NO: 18 - DNA, 60 bp, synthetic construct
  misc_feature 1..60: note = oligoA-sgRNA2
  source 1..60: mol_type = other DNA
ctaatacgactcactataggtgatgcggccttccactgcgttttagagctagaaatagca

SEQ ID NO: 19 - DNA, 80 bp, synthetic construct
  misc_feature 1..80: note = oligoB
  source 1..80: mol_type = other DNA
aaaagcaccgactcggtgccactttttcaagttgataacggactagccttattttaacttgctatttctagctctaaaac

SEQ ID NO: 20 - DNA, 20 bp, synthetic construct
  misc_feature 1..20: note = oligoC
  source 1..20: mol_type = other DNA
aaaagcaccgactcggtgcc

SEQ ID NO: 21 - DNA, 23 bp, synthetic construct
  misc_feature 1..23: note = Target sequence on exon 1_sgNRA1
  source 1..23: mol_type = other DNA
caggatgttcaccaaagtgacgg

SEQ ID NO: 22 - DNA, 23 bp, synthetic construct
  misc_feature 1..23: note = Target sequence on exon 9_sgRNA2
  source 1..23: mol_type = other DNA
gtgatgcggccttccactgcagg

SEQ ID NO: 23 - DNA, 23 bp, synthetic construct
  misc_feature 1..23: note = Target sequence on exon 6_Pm-Nt5-sgRNA
  source 1..23: mol_type = other DNA
tcaggaggtgggaaagactctgg

SEQ ID NO: 24 - DNA, 23 bp, synthetic construct
  misc_feature 1..23: note = Target sequence on exon 1_sgRNA-t
  source 1..23: mol_type = other DNA
ccctgcttcgctggtgtggccag

SEQ ID NO: 25 - DNA, 1851 bp, Clarias garienpinus
  source 1..1851: mol_type = genomic DNA; note = nt5e gene of Clarias garienpinus
atgcgctactcgacagcggcgcgcgcgctgctccctttgctggtagtgtgcgcgagcttgcggctcgcggcggcagactgggagctgacgctcttgcacaccaacgacgtgcacgcgcgggtcgaggagaccaacaaggactcgggcaagtgcacgaagggagagtgcttcgccggtgtcgcgcggcgttccacgaagatcaaagagatccggagcaaggagaagaacgtgctgctgctggacgcaggggatcagtttcagggcaccgtctggttcaacgtctacaaaggcgatgaggcggcgcacttcatgaacaaactccaatacgatgccatggctttgggaaaccacgagtttgacaatagagtggaaggtctcatacccttcctgcagaaggtgaagtgtcctgtactcagcgctaacatcaaggcggttgagcctgttgcgtcaaatattagcggatatttttcaccttataaaatcttaaatgtaacctctgagaaagtgggtatagtgggatacactaccaaggagactccagctctgtcattgccagggccgtacctgcattttgaggacgaggtcgtagccgtgcagcgggaggtagacaaactcatcgctctcggcgttaacaagatcatcgccctcggccattctggatttgacacggacaaagagatcgccaaaaaggtccgtggagtggacgtggtgattggaggacacaccaacacttttttatacacaggggctcccccatccagcgaggttccagcaggaccctacccattaatggtacagtccgatgatggtcgcaaagtgccagtggtccaggcgtacgcctttgggaaataccttggctatctcaaagtgactttcgattctgctggaaacgtgataaaagcggaaggaaatccaatcctcctaaatagcagcgtaaccgaagatcccagcattaaggcagatgtggatacctggaaggtcaaattggccaattactcagctcagtttgtgggaaacactctggtctacctgaacgggacatttgaagagtgtcgatttcgggaatgcaacctgggaaacttgatctgcgatgccatggtccatcactacatcaaatatccggatgaactccagtggaaccatgttagttcctgtattttaaacggagggggcatacgatcctccattgatgagagaagcagaaatggtagcatcactatggaggacgtgttgaccgtgctgccgtttggaggaacatatgatctggttcaactgaatggctcgactcttctccaggccttcgagcacagcgtccacagatatggaggaaacactggggagttcctacaggtctcaggctttcagctggagtacgacctcaccaagccctctggtcatcgtgtgatcaaggcgagagtgttatgtaccgagtgtcgagtgccacactacgagcccctggacgctacaaaagtctacagggtggtgatgccatcctacctagtggacggaggagacggtttctcaatgatcaagcagcagatcctgaaacatgatagtggtgacctcgacatatccgtcttttctcgctacatcacagagaggcagagggttcacccatccgtggaaggacgaatccgtctcctaaattctgccgtggaaatctcaaactcttcctcctacaccctctgcgtcgtgattctcattattgtgggtgttatgctcattatgttggctggtgtctactactacagaaggaaaggtcttgttgatgataaggttacagtacagtacagaagagctatagatggagatgaaaaactgaatgaccaaaccacagtaacgagttcctga

SEQ ID NO: 26 - protein, 616 aa, Clarias garienpinus
  source 1..616: note = NT5E of Clarias garienpinus
MRYSTAARALLPLLVVCASLRLAAADWELTLLHTNDVHARVEETNKDSGKCTKGECFAGVARRSTKIKEIRSKEKNVLLLDAGDQFQGTVWFNVYKGDEAAHFMNKLQYDAMALGNHEFDNRVEGLIPFLQKVKCPVLSANIKAVEPVASNISGYFSPYKILNVTSEKVGIVGYTTKETPALSLPGPYLHFEDEVVAVQREVDKLIALGVNKIIALGHSGFDTDKEIAKKVRGVDVVIGGHTNTFLYTGAPPSSEVPAGPYPLMVQSDDGRKVPVVQAYAFGKYLGYLKVTFDSAGNVIKAEGNPILLNSSVTEDPSIKADVDTWKVKLANYSAQFVGNTLVYLNGTFEECRFRECNLGNLICDAMVHHYIKYPDELQWNHVSSCILNGGGIRSSIDERSRNGSITMEDVLTVLPFGGTYDLVQLNGSTLLQAFEHSVHRYGGNTGEFLQVSGFQLEYDLTKPSGHRVIKARVLCTECRVPHYEPLDATKVYRVVMPSYLVDGGDGFSMIKQQILKHDSGDLDISVFSRYITERQRVHPSVEGRIRLLNSAVEISNSSSYTLCVVILIIVGVMLIMLAGVYYYRRKGLVDDKVTVQYRRAIDGDEKLNDQTTVTSS

SEQ ID NO: 27 - DNA, 23 bp, synthetic construct
  source 1..23: mol_type = other DNA; note = sgRNA-p
cttcaacggaggcggcgtccggg

(19) United States
(12) Patent Application Publication
(10) Pub. No.: US 2025/0160306 A1
(43) Pub. Date: May 22, 2025

(54) FISH, METHOD FOR PRODUCING FISH, AND METHOD FOR PRODUCING FISH EXHIBITING ACCELERATED MATURATION

(71) Applicants: KYOTO UNIVERSITY, Kyoto-shi, Kyoto (JP); KINKI UNIVERSITY, Higashiosaka-shi, Osaka (JP); REGIONAL FISH INSTITUTE, LTD., Kyoto-shi, Kyoto (JP)

(72) Inventors: Masato KINOSHITA, Kyoto-shi, Kyoto (JP); Keitaro KATO, Wakayama (JP); Youhei WASHIO, Wakayama (JP); Yu MURAKAMI, Nara-shi Nara (JP); Kenta KISHIMOTO, Kyoto-shi, Kyoto (JP); Yuki HONDA, Kyoto-shi, Kyoto (JP); Ricardo Shohei HATTORI, Kyoto-shi, Kyoto (JP)

(21) Appl. No.: 18/839,916

(22) PCT Filed: Jun. 14, 2023
(86) PCT No.: PCT/JP2023/022090

§ 371 Date: Aug. 20, 2024

(30) Foreign Application Priority Data
Jun. 15, 2022 (JP) 2022-096417

(51) Int. Cl. A01K 67/0276; C12N 9/16

(52) U.S. Cl. CPC A01K 67/0276; C12N 9/16; C12Y 301/03005; A01K 2217/075; A01K 2227/40

(57) ABSTRACT

Provided is a fish in which accumulation of inosinic acid is enhanced or accelerated during ripening. In a fish of the present disclosure, the function of the ecto-5′-nucleotidase (nt5e) gene is lost.

### SEQUENCE LISTING

The present application is being filed along with a Sequence Listing in electronic format. The Sequence Listing is provided as a file entitled Sequence Listing 0021.xml created on Aug. 19, 2024, which is 46 KB in size. The information in the electronic format of the Sequence Listing is incorporated herein by reference in its entirety.

### TECHNICAL FIELD

This disclosure relates to a fish, a fish production method, and a method for producing an accelerated ripened fish.

### BACKGROUND ART

After the death of a fish, nucleic acids in the muscle of the fish degrade. It is known that the inosinic acid content in the muscle increases with this degradation (Patent Literature 1).

### CITATION LIST

### Patent Literature

Patent Literature 1: JP S61-115499A

### SUMMARY OF INVENTION

### Technical Problem

Because the umami flavor of fish meat originates in inosinic acid, it is possible to improve the umami flavor by ripening the fish after their death. Meanwhile, the muscle of a fish becomes soft after the death of the fish. Accordingly, it is currently difficult to obtain fish that maintains a firm texture and has umami flavor.

Therefore, it is an object of the present disclosure to provide a fish in which accumulation of inosinic acid is enhanced or accelerated during ripening.

### Solution to Problem

In order to achieve the object above, a fish of the present disclosure (hereinafter also referred to as a “first fish”) has a loss of function of the ecto-5′-nucleotidase (nt5e) gene.

The present disclosure is directed to a portion of the fish of the present disclosure.

A fish production method of the present disclosure (hereinafter also referred to as a “first production method”) includes a step (a) below:
(a) a mating step of mating the fish of the present disclosure with another fish.

A production method of the present disclosure is a method for producing an accelerated ripened fish (hereinafter also referred to as a “second production method”), the method including

a functional loss-causing step of causing loss of the function of the ecto-5′-nucleotidase (nt5e) gene of a target fish.

An enhancing method of the present disclosure is a method for enhancing an inosinic acid content during ripening of fish meat, the method including

a ripening step of ripening fish meat,

wherein the fish meat is fish meat of the fish of the present disclosure and/or fish meat in an edible portion of the fish of the present disclosure.

A method for screening an accelerated ripened fish according to the present disclosure (hereinafter also referred to as a “screening method”) includes a selection step of selecting, from a test sample of fish, a test fish in which the function of the ecto-5′-nucleotidase (nt5e) gene is lost as an accelerated ripened fish.

A fish production method of the present disclosure (hereinafter also referred to as a “third production method”) includes a screening step of screening a test sample of fish to find a test fish in which the function of the ecto-5′-nucleotidase (nt5e) gene is lost,

wherein the screening step is conducted in accordance with the screening method of the present disclosure.

A fish (hereinafter also referred to as a “second fish”) of the present disclosure is obtained using the first production method, the second production method, or the third production method of the present disclosure.

A method for detecting an ability of a fish to be accelerated ripened according to the present disclosure (hereinafter also referred to as a “detection method”) includes a detection step of detecting if the function of the ecto-5′-nucleotidase (nt5e) gene is lost in a test fish.

Processed food of the present disclosure contains the fish of the present disclosure.

### Advantageous Effects of Invention

With the present disclosure, it is possible to provide a fish in which accumulation of inosinic acid is enhanced or accelerated during ripening.

### BRIEF DESCRIPTION OF DRAWINGS

FIG. 1 shows graphs illustrating the inosinic acid levels in medakas in Example 1 in which the function of the nt5e gene is lost.

FIG. 2 is a graph illustrating the inosinic acid levels in red seabreams in Example 2 in which the function of the nt5e gene is lost.

FIG. 3A and FIG. 3B show graphs illustrating the inosinic acid levels and the K values of tilapias in Example 3 in which the function of the nt5e gene is lost.

FIG. 4A and FIG. 4B show graphs illustrating the inosinic acid levels and the K values of bastard halibuts in Example 4 in which the function of the nt5e gene is lost.

### DESCRIPTION OF EMBODIMENTS

### Definitions

The term “fish” as used herein means an animal that is classified into an animal group consisting of the Vertebrata excluding the Tetrapoda.

The term “loss of function” as used herein means, for example, a state in which the inherent function of a target gene decreases or is lost.

The term “loss-of-function mutation” as used herein means a mutation that leads to a (significant) reduction in the inherent function of a target gene and/or complete loss of function.

The above-mentioned “mutation that leads to complete loss of function” can also be referred to as, for example, “null mutation” or “amorph”.

The term “fish” as used herein means an individual fish.

The term “a portion of a fish” as used herein means a part or portion of an individual fish.

The term “ripening” as used herein means a step or process of degrading proteins into amino acids in a target fish. The ripening can also be referred to as, for example, “aging”.

The phrases “activity of degrading inosinic acid” or “inosinic acid degrading activity” as used herein means activity of degrading inosinic acid into inosine.

Information on the sequences of proteins described in this specification or the sequences of nucleic acids (e.g., DNAs or RNAs) encoding the proteins is available from Protein Data Bank, UniProt, Ensembl, GenBank, or the like. In addition, the nucleic acid sequences of RNAs can be obtained from the nucleic acid sequences of the corresponding DNAs using sequence conversion software as appropriate.

### <Fish>

In a certain aspect, the present disclosure provides a fish in which accumulation of inosinic acid is enhanced or accelerated during ripening. In a fish (first fish) of the present disclosure, the function of the ecto-5′-nucleotidase (nt5e) gene is lost.

As a result of extensive research, the inventors of the present invention have found that ecto-5′-nucleotidase (NT5E) of a fish contributes to changes in the contents of umami components produced after the death of the fish. In addition, as a result of further research, the inventors of the present invention have determined that NT5E contributes to degradation of an umami component inosinic acid and found that causing loss of the function of the ecto-5′-nucleotidase (nt5e) gene makes it possible to accelerate accumulation of inosinic acid, and thus the present disclosure was achieved. With the present disclosure, it is possible to shorten the time required for the content of inosinic acid to reach a certain level as compared to that of a fish having the wild-type (normal) nt5e gene. In general, a fish becomes soft immediately after the death, and the eating texture becomes soft. Accordingly, in the case of the fish above, it is difficult to obtain a fish that maintains a firm texture and has umami flavor. Meanwhile, with the present disclosure, the accumulation of inosinic acid in fish meat can be accelerated after the death of a fish, and therefore, it is expected that a fish that maintains a firm texture and has umami flavor is obtained.

Examples of the fish above include fish belonging to the family Tetraodontidae (puffers), the family Ostraciidae (boxfishes), the family Sparidae (sea breams and porgies), the family Salmonidae, the family Cyprinidae, the family Serranidae (sea basses), the family Cichlidae, the family Oryziidae (medakas), the family Paralichthys, the family Carangidae, the family Bagridae, the family Clariidae, the family Intaluridae, and the like.

Examples of the fish belonging to the family Tetraodontidae above include fish belonging to the genus Takifugu such as a tiger puffer (Takifugu rubripes), a purple puffer (Takifugu porphyreus), and a grass puffer (Takifugu niphobles); and fish belonging to the genus Lagocephalus such as a half-smooth golden puffer (Lagocephalus wheeleri).

Examples of the fish belonging to the family Ostraciidae above include fish belonging to the genus Ostracion such as a black-spotted boxfish (Ostracion immaculatus).

Examples of the fish belonging to the family Sparidae above include fish belonging to the genus Pagrus such as a red seabream (Pagrus major) and an Australasian snapper (Pagrus auratus); fish belonging to the genus Acanthopagrus such as a Japanese black porgy (Acanthopagrus schlegeli) and a yellowfin seabream (Acanthopagrus latus); fish belonging to the genus Dentex such as a yellowback seabream (Dentex tumifrons); and fish belonging to the genus Sparus such as a gilt-head bream (Sparus aurata).

Examples of the fish belonging to the family Salmonidae above include fish belonging to the genus Oncorhynchus such as a rainbow trout (Oncorhynchus mykiss), a chinook salmon (Oncorhynchus tshawytscha), a cherry trout (Oncorhynchus masou), a red spotted masu trout (Oncorhynchus masou), a black kokanee (Oncorhynchus kawamurae), a pink salmon (Oncorhynchus gorbuscha), and a chum salmon (Oncorhynchus keta); fish belonging to the genus Salmo such as a brown trout (Salmo trutta), a red salmon (Oncorhynchus nerka), a silver salmon (Oncorhynchus kisutch), and an Atlantic salmon (Salmo salar); fish belonging to the genus Salvelinus such as a dolly varden (Salvelinus malma), a whitespotted char (Salvelinus leucomaenis), a brook trout (Salvelinus fontinalis), and a lake trout (Salvelinus namaycush); and fish belonging to the genus Parahucho such as a Japanese huchen (Parahucho perryi).

Examples of the fish belonging to the family Cyprinidae above include fish such as a honmoroko (Gnathopogon caerulescens), silver carp (Hypophthalmichthys molitrix), carp (Cyprinus carpio), grass carp (Ctenopharyngodon idellus), bighead carp (Hypophthalmichthys nobilis), crucian carp (Carassius carassius), a catla (Cyprinus catla), black carp (Mylopharyngodon piceus), mud carp (Cirrhinus molitorella), mrigal carp (Cirrhinus cirrhosus), a catla (Catla catla), a rohu (Labeo rohita), and a wuchang bream (Megalobrama amblycephala).

Examples of the fish belonging to the family Serranidae above include fish belonging to the genus Epinephelus such as a convict grouper (Epinephelus septemfasciatus), a longtooth grouper (Epinephelus bruneus), a redspotted grouper (Epinephelus akaara), a malabar grouper (Epinephelus malabaricus), a white grouper (Epinephelus aeneus), a banded grouper (Epinephelus amblycephalus), an areolate grouper (Epinephelus areolatus), a duskytail grouper (Epinephelus bleekeri), a palemargin grouper (Epinephelus bontoides), (Epinephelus chlorostigma), an orange-spotted grouper (Epinephelus coiodes), a blacktip grouper (Epinephelus fasciatus), a brown-marbled grouper (Epinephelus fuscoguttatus), a starry grouper (Epinephelus labriformis), a giant grouper (Epinephelus lanceolatus), a highfin grouper (Epinephelus maculatus), a Malabar grouper (Epinephelus malabricus), a dusky grouper (Epinephelus marginatus), a white-streaked grouper (Epinephelus ongus), a camouflage grouper (Epinephelus polyphekadion), a longfin grouper (Epinephelus quoyanus), a sixbar grouper (Epinephelus sexfasciatus), a Nassau grouper (Epinephelus striatus), a greasy grouper (Epinephelus tauvina), and a potato grouper (Epinephelus tukula); fish belonging to the genus Cromileptes such as a humpback grouper (Cromileptes altivelis); fish belonging to the genus Plectropomus such as a leopard coral grouper (Plectropomus leopardus); and crossbreeds between fish belonging to the family Serranidae.

Examples of the fish belonging to the family Cichlidae above include fish belonging to the genus Oreochromis such as a Nile tilapia (Oreochromis niloticus), a Mozambique tilapia (Oreochromis mossambicus), and a blue tilapia (Oreochromis aureus).

Examples of the fish belonging to the family Oryziidae above include fish belonging to the genus Oryzias such as a medaka (Oryzias latipes, Oryzias sakaizumii) and a Javanese medaka (Oryzias javanicus).

Examples of the fish belonging to the family Paralichthyidae above include fish belonging to the genus Paralichthys such as a bastard halibut (Paralichthys olivaceus).

Examples of the fish belonging to the family Carangidae above include fish belonging to the genus Seriola such as a yellowtail amberjack (Seriola lalandi) and a greater amberjack (Seriola dumerili).

Examples of the fish belonging to the family Bagridae above include fish belonging to the genus Pelteobagrus such as yellowhead catfish (Pseudobagrus fulvidraco).

Examples of the fish belonging to the family Clariidae above include fish belonging to the genus Clarias such as a whitespotted clarias (Clarias garienpinus).

Examples of the fish belonging to the family Intaluridae above include fish belonging to the genus Ictalurus such as a channel catfish (Ictalurus punctatus).

In the present disclosure, the fish may be a true breed or crossbreed. The crossbreed may be a crossbreed obtained through intergeneric crossing.

In the present disclosure, it is preferable that the fish is, for example, a fish for aquaculture.

In the present disclosure, the fish may be a saltwater fish, a freshwater fish, or a brackish fish.

In the present disclosure, the growth stage of the fish is not particularly limited and may be any of a larval fish (larva), a juvenile fish, an immature fish (a fry fish, a young fish), and an adult fish.

In general, the ecto-5′-nucleotidase (nt5e) is known as a protein having activity of catalyzing chemical reaction to convert an extracellular 5′-ribonucleotide into a ribonucleoside.

Regarding the nt5e gene and a homologous gene thereof, two or more types are present on the genome of a fish.

In the present disclosure, it is sufficient that the nt5e gene of the fish is a gene encoding the ecto-5′-nucleotidase present in a fish (wild-type nt5e gene), and specific examples of such an nt5e gene include nt5e genes listed in Table 1 below.

TABLE 1

| Species | Scientific | | Gene ID |
|---|---|---|---|
| name | name | Gene ID (Ensembl) | (NCBI) |
| Japanese | Oryzias latipes | ENSORLG00000014932 | 101174549 |
| medaka | | | |
| Tiger puffer | Takifugu | ENSTRUG00000004739 | 101065733 |
| | rubripes | | |
| Blue tilapia | Oreochromis | ENSOABG00000015619 | — |
| | aureus | | |
| Nile tilapia | Oreochromis | ENSONIG00000003636 | — |
| | niloticus | | |
| Gilt-head | Sparus aurata | ENSSAUG00010026291 | 115566087 |
| bream | | | |
| European | Dicentrarchus | ENSDLAG00005000098 | — |
| seabass | labrax | | |
| Yellowtail | Seriola lalandi | ENSSLDG00000003971 | 111644776 |
| amberjack | dorsalis | | |
| Greater | Seriola | ENSSDUG00000017875 | 111225877 |
| amberjack | dumerili | | |
| Rainbow | Oncorhynchus | ENSOMYG00000034004 | 110498125 |
| trout | mykiss | | |
| Chinook | Oncorhynchus | ENSOTSG00005034677 | 112262034 |
| salmon | tshawytscha | | |
| Brown trout | Salmo trutta | ENSSTUG00000051027 | 115172841 |
| Atlantic | Salmo salar | ENSSSAG00000015918 | 106565054 |
| salmon | | | |
| Bastard | Paralichthys | — | 109646855 |
| halibut | olivaceus | | |
| Channel | Ictalurus | — | 108279846 |
| catfish | punctatus | | |
| Yellowhead | Tachysurus | — | 113662406 |
| catfish | fulvidraco | | |

(—: not available)

When the fish is a red seabream, a tiger puffer, a tilapia, a rainbow trout, a honmoroko, a bastard halibut, a whitespotted clarias, or a medaka, specific examples of the nt5e gene (wild-type nt5e gene) of the fish include polynucleotides of (Pn), (Pt), (Po), (Pm), (Pg), (Pp), (Pq), and (Pl) below, or genome regions encoding these polynucleotides. Note that the base sequences of SEQ ID NOs: 1, 3, 5, 7, 9, 11, 13, and 25 below include a stop codon.
(Pn) Any of polynucleotides (Pn1) to (Pn7) below:
(Pn1) a polynucleotide having a base sequence of SEQ ID NO: 1;
(Pn2) a polynucleotide that encodes a protein having an activity of degrading inosinic acid and that has a base sequence consisting of the base sequence of (Pn1) above with a deletion, a substitution, an insertion, and/or an addition of one or several bases;
(Pn3) a polynucleotide that encodes a protein having an activity of degrading inosinic acid and that has a base sequence having 80% or more identity to the base sequence of (Pn1) above;
(Pn4) a polynucleotide that encodes a protein having an activity of degrading inosinic acid and that has a base sequence complementary to a polynucleotide capable of hybridizing to the polynucleotide having the base sequence of (Pn1) above under a stringent condition;
(Pn5) a polynucleotide encoding a protein having an amino acid sequence of SEQ ID NO: 2;
(Pn6) a polynucleotide encoding a protein that has an activity of degrading inosinic acid and that has an amino acid sequence consisting of the amino acid sequence of SEQ ID NO: 2 with a deletion, a substitution, an insertion, and/or an addition of one or several amino acids; and
(Pn7) a polynucleotide encoding a protein that has an activity of degrading inosinic acid and that has an amino acid sequence having 80% or more identity to the amino acid sequence of SEQ ID NO: 2.

Base Sequence of nt5e Gene of Red Seabream (SEQ ID NO: 1)

5′-ATGGGAGCGCTGCGCCCGCGCTGCCTCCACCTCCTCCTCCTGCTGCTGGGCTTCTCCG
TGTCCACCTCGGCGGCCTGGGACCTGGTTCTCCTCCACACCAACGACGTGCACGCCC
GGGTGGAGGAGACCAGCAAGCACTCCGGGAAATGCAGCAGCAGCAGGAAGAGCGG
CGGGTGTTTCGCCGGAGTGGCCCGCAGAGCCACGATGATCAAGAAGATCCGCTCCAC
CGACAGCAACGTGCTGCTGCTGGACGCCGGGGACCAGTTCCAGGGCAGCGTGTGGT
TCAATTACTACAAGGGAGCCGAGGCTGCTCACTTCATGAACAAACTGCAGTATGATG
CCATGGCCTTGGGAAATCACGAGTTCGACAACGGAGTGGAAGGACTCATGAAGCCG
TTCATGGAGAAGATCAGGTGTCCTGTTCTGAGCGCCAACATCAAACCTGATGAAGCT
ATGGCACCGACGTTCAGCAGCTCCTATCTACCTTATAAGATACTGACTGTGGGCAGCG
AGAAGGTGGGCGTAGTGGGATACACGTCGCAGGAAACTCCGGCTCTGTCCAGACCT
GGACCTCACCTGGAGTTTGAGGACGAGGTGACCTCGCTGCAGCTGCAGGTGAACAA
ACTGCAGACGCTGGGAGTGAATAAGATCATCGCTCTGGGACACTCCGGCTTCACGGT
GGATCGCGAGATCGCCAAGAAGGTCCGCGGAGTCGACGTGGTCATCGGTGGACACA
CCAACACTTTCCTGTTCACAGGGCATCCTCCCTCCTCTGAGGTCCCGTTGGGTAGTTA
TCCGTTCATGGTGACATCAGTGGACGGGCGCCAGGTTCCTGTCGTGCAGGCCTACGC
CTTTGGAAAATATCTGGGTCACTTGAAAGTGACCTTTGATGATGCTGGAAACGTGATG
AAGTCAACAGGAAACCCAATCCTGCTGGACAGCAGCGTCCCACAGGATCCAGATGTT
CTTGCTGACGTGGAGGAATGGAAAAAGAGTCTGGCCAACTACTCAGCTCAGGAGGT
GGGAAAGACTCTGGTGTTCCTGAACGGGACCACTGAGGAGTGTCGATTTCGAGAATG
CAACCTGGGAAACCTGATCTGTGACGCCATGGTCAACAACAACATCCGATTCTTAGA
GGACGAGCAGTGGAACCACGTCAGCGCCTCCATCTTCAATGGAGGAGGCATCCGAA
CATCCATCGATGAGCACAGCAGGAACGGTTCGATCACCATGGAGGACCTGATCTCCG
TCTTACCTTTCGGAGGAACCTTCGACCTGGTGCAGCTGAACGGCTCCACGCTGAGGA
GAGCTTTCGAGCACTCAGTGAAACGATACGGCGAAAGCACCGGAGAATTTCTCCAA
GTGTCCGGTTTCCATGTAGAGTTCGACCTCTCCAAACCGGCTGGTAGTCGCGTGAGG
AGCCTCGACATCCTCTGCACTCAGTGTCGAGTCCCTCAATACGAACCCGTGGAGGAT
GAGACGGTTTACACAGTGGTGGTGCCGTCCTTCATGGTGACAGGCGGAGACGGATAC
TCCATGATCAGGAATGAGACGCTCAAACACAACAGCGGTAATCTGGACATTTCAGTC
GTGTCCAACTACATCATGCAGAGAAAGAGAGTTTATCCGGCTGTTGAAGGACGAATC
AAGATCTACAACTCAGCTTCTGGACCGCGAGGACAAATTTTACTGGTTTCACTGGTGC
TGCTCTGGACTCTGTGGGAGCATGTAGGAGTGACTTCAACCAGTTTTTAA-3′

In (Pn1) above, the base sequence of SEQ ID NO: 1 is the base sequence encoding the amino acid sequence of (Pn5) above. The base sequence of SEQ ID NO: 1 can be obtained from, for example, a red seabream (Pagrus major).

In (Pn2) above, the “one or several” bases may be any number of bases as long as, for example, the protein encoded by the polynucleotide of (Pn2) above has the inosinic acid degrading activity. The “one or several” bases in (Pn2) above refer to, for example, 1 to 351 bases, 1 to 263 bases, 1 to 175 bases, 1 to 87 bases, 1 to 70 bases, 1 to 52 bases, 1 to 35 bases, 1 to 17 bases, 1 to 8 bases, 1 to 6 bases, 1 to 3 bases, 1 or 2 bases, or 1 base, in the base sequence of (Pn1) above. In the present disclosure, the numerical range of the number of bases, amino acids, or the like discloses, for example, all positive integers belonging to that range. Specifically, for example, “1 to 5” means disclosure of all of “1, 2, 3, 4, and 5” (hereinafter the same applies).

In (Pn3) above, the “identity” may be any degree of identity as long as, for example, the protein encoded by the polynucleotide of (Pn3) above has the inosinic acid degrading activity. In (Pn3) above, the identity to the base sequence of (Pn1) above is, for example, 80% or more, 85% or more, 90% or more, 95% or more, 96% or more, 97% or more, 98% or more, or 99% or more. The “identity” can be determined through alignment of two base sequences or two amino acid sequences (hereinafter the same applies). The above-mentioned alignment can be calculated using, for example, BLAST, FASTA, or the like with default parameters.

The “polynucleotide capable of hybridizing” in (Pn4) above is not limited as long as the protein encoded by the polynucleotide of (Pn4) above has the inosinic acid degrading activity. In (Pn4) above, the “polynucleotide capable of hybridizing” is, for example, a polynucleotide that is fully or partially complementary to the polynucleotide of (Pn1) above. The hybridization can be detected, for example, through various hybridization assays. There is no particular limitation on the above-mentioned hybridization assays, and methods described in, for example, Sambrook et al. ed. “Molecular Cloning: A Laboratory Manual 2^{nd} Ed.” (Cold Spring Harbor Laboratory Press (1989)), etc. can also be employed.

In (Pn4) above, the “stringent condition” may be, for example, any of a low-stringent condition, a medium-stringent condition, and a high-stringent condition. The “low-stringent condition” is, for example, a condition where 5×SSC, 5×Denhardt's solution, 0.5% SDS, and 50% formamide are used, and the temperature is set to 32° C. The “medium-stringent condition” is, for example, a condition where 5×SSC, 5×Denhardt's solution, 0.5% SDS, and 50% formamide are used, and the temperature is set to 42° C. The “high-stringent condition” is, for example, a condition where 5×SSC, 5×Denhardt's solution, 0.5% SDS, and 50% formamide are used, and the temperature is set to 50° C. A person skilled in the art can determine the degree of stringency by, for example, selecting conditions such as the temperature, the salt concentration, the concentration and length of a probe, the ionic strength, and the time as appropriate. The conditions described in, for example, Sambrook et al. ed. “Molecular Cloning: A Laboratory Manual 2^{nd} Ed.” (Cold Spring Harbor Laboratory Press (1989)), etc. described above can also be employed as the “stringent condition”.

The polynucleotide of (Pn5) above may have any base sequence as long as, for example, the protein encoded by the polynucleotide of (Pn5) above has the inosinic acid degrading activity. The base sequence of the polynucleotide of (Pn5) above can be designed through, for example, conversion to corresponding codons based on the amino acid sequence of SEQ ID NO: 2.

Amino Acid Sequence of NT5E Protein of Red Seabream (SEQ ID NO: 2)

MGALRPRCLHLLLLLLGFSVSTSAAWDLVLLHTNDVHARVEETSKHSGK
CSSSRKSGGCFAGVARRATMIKKIRSTDSNVLLLDAGDQFQGSVWFNYY
KGAEAAHFMNKLQYDAMALGNHEFDNGVEGLMKPFMEKIRCPVLSANIK
PDEAMAPTFSSSYLPYKILTVGSEKVGVVGYTSQETPALSRPGPHLEFE
DEVTSLQLQVNKLQTLGVNKIIALGHSGFTVDREIAKKVRGVDVVIGGH
TNTFLFTGHPPSSEVPLGSYPFMVTSVDGRQVPVVQAYAFGKYLGHLKV
TFDDAGNVMKSTGNPILLDSSVPQDPDVLADVEEWKKSLANYSAQEVGK
TLVFLNGTTEECRFRECNLGNLICDAMVNNNIRFLEDEQWNHVSASIFN
GGGIRTSIDEHSRNGSITMEDLISVLPFGGTFDLVQLNGSTLRRAFEHS
VKRYGESTGEFLQVSGFHVEFDLSKPAGSRVRSLDILCTQCRVPQYEPV
EDETVYTVVVPSFMVTGGDGYSMIRNETLKHNSGNLDISVVSNYIMQRK
RVYPAVEGRIKIYNSASGPRGQILLVSLVLLWTLWEHVGVTSTSF

In (Pn6) above, the “one or several” amino acids in the amino acid sequence may be any number of amino acids as long as, for example, the protein encoded by the polynucleotide of (Pn6) above has the inosinic acid degrading activity. The “one or several” amino acids in (Pn6) above refer to, for example, 1 to 116 amino acids, 1 to 86 amino acids, 1 to 58 amino acids, 1 to 29 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 2.

In (Pn7) above, the “identity” of the amino acid sequence may be any degree of identity as long as, for example, the protein encoded by the polynucleotide of (Pn7) above has the inosinic acid degrading activity. In (Pn7) above, the identity to the amino acid sequence of SEQ ID NO: 2 is, for example, 80% or more, 85% or more, 90% or more, 95% or more, 96% or more, 97% or more, 98% or more, or 99% or more.
(Pt) Any of polynucleotides (Pt1) to (Pt7) below:
(Pt1) a polynucleotide having a base sequence of SEQ ID NO: 3;
(Pt2) a polynucleotide that encodes a protein having an activity of degrading inosinic acid and that has a base sequence consisting of the base sequence of (Ptl) above with a deletion, a substitution, an insertion, and/or an addition of one or several bases;
(Pt3) a polynucleotide that encodes a protein having an activity of degrading inosinic acid and that has a base sequence having 80% or more identity to the base sequence of (Pt1) above;
(Pt4) a polynucleotide that encodes a protein having an activity of degrading inosinic acid and that has a base sequence complementary to a polynucleotide capable of hybridizing to the polynucleotide having the base sequence of (Pt1) above under a stringent condition;
(Pt5) a polynucleotide encoding a protein having an amino acid sequence of SEQ ID NO: 4;
(Pt6) a polynucleotide encoding a protein that has an activity of degrading inosinic acid and that has an amino acid sequence consisting of the amino acid sequence of SEQ ID NO: 4 with a deletion, a substitution, an insertion, and/or an addition of one or several amino acids; and
(Pt7) a polynucleotide encoding a protein that has an activity of degrading inosinic acid and that has an amino acid sequence having 80% or more identity to the amino acid sequence of SEQ ID NO: 4.

Base Sequence of nt5e Gene of Tiger Puffer (SEQ ID NO: 3)

5′-ATGAGCGTGTGGTCGCGCCGGTGCGCTCTCTGGGTCTGCATCTCTCTACTGGCGGGG
CCAGTTTGGACCTTCGAGCTGACGCTGCTTCACACCAACGACAACCATGCGCGGATC
GAGGAGACCAGCGAGGACTTGGGAAAGTGCTCATCCAGGGGTCCCTGTTTCGCGGG
GGTCGCCCGGAGGTTCACCAAAGTGTCGGAGATCCGCAAAAAGGAAAAGAACGTTT
TGTTCCTGGACGCTGGGGACCAATTTCAGGGGACGGTTTGGTTTAACTACTACAAAG
GCGCAGAAGCCGCGCACTTTATGAATAAACTTGGTTATAATGCGATGGCTTTAGGGAA
CCATGAGTTTGATAATGGGGTGGAGGGACTCCTGCCTTTCCTCCAGAATGTGAACTGT
TCTGTGGTGAGTGCGAACATCCAACCTGACCAGAGTCTGGCTGCAAAGCTCAGCGGT
TTCCTCCAGCCCTACACGGTCCTCAACGTGGGCTCAGAGAAGGTGGCCGTGGTGGGC
TACACCACCGCCGAGACCCCCTTCCTATCTATGCCAGGCCCACATCTTAAATTTAACG
AGGAAGTGGAGGCGCTCCAGGTCCAGGTGGACAAACTGGAAACCCTGGGTTATGAT
AAGATCATCGCCTTGGGTCACTCCGGCTTTGATGTGGATCAGCAGATCGCCAAGCGA
GTGAGAGGGGTTGACGTCGTCATCGGAGGACACACCAACACCTTCCTTTATACTGGA
AAAGCCCCGTCCACTGAAGTGCCTGTAGGTCCTTATCCTTTTATAGTGAGGTCTGACG
ATGGGAGAAACGTCCCGGTGGTCCAAGCTTTTGCCTTTGGAAAATACCTCGGTTACTT
GAGGGTCACATTTGACGATGCTGGAAAAGTAATCAAGGCTGCAGGAAACCCAATCCT
ACTGGACAGCAGCGTCCCTCAGGATCCGGACGTCCTGGCTGAGGTCAACAGGTGGA
AGAAAGACCTGGCTCAGTACTCCTCACAGTATGTCGGGCAAACCTTAGTTTATCTCAA
CGGCACGTTTGAAGAGTGCCGATTTCGGGAGTGTAACCTCGGCAACCTGATCTGCGA
TGGCATGATCGACCACAACATCAAATTCTCCAGCGAGCTGCAGTGGAACCACGTGAG
CCTTTGTATGCTGAACAGCGGAGCCATACGGGCTCCCATCGATGAGCGCTACAAGAA
CGGCTCCATAACTATGGAGGACGTCCTCACTGTCTTGCCGTTCGGAGGGACTGTTGAC
TTGGTGCAGATAAAAGGGTCAACGGTGAAGAAGGCGTTCGAACATGCCGTCCACAG
ATTTGGAAGCATGTCAGGAGAGTTTCTGCAAGTCTCAGGCTTCCATGTCAAGTACGA
CCTGTCCAAACCAGTGAACCAACGCGTCACGTCTCTGTCGGCACTTTGCACTGAGTG
CCGCGTACCCAAATATGAGCCCATAGACCCGGAGAGGACGTACAAGGTGACCATGCC
GTCATATCTGGTGGACGGTGGTGACGGTTTCAGCATGATCAAGGAGGAGTTACTGAA
GCATAACACAGGTGACTTGGACATTTCTGTGTTCTCTAAATATATCTCGCAGCAGAAG
CGCGTATATCCAGCAGTGGAGGGCCGGATCACAGTCAGGGGCTCGGCCTCCTCTGCA
GCCCACAGCCTCGACGTATTCCTTCATACTTGGCCTCTTCTCCCATACATTTCCCCAGG
AAGTCTGAAAGATAAATGGCTATAG-3′

In (Pt1) above, the base sequence of SEQ ID NO: 3 is the base sequence encoding the amino acid sequence of (Pt5) above. The base sequence of SEQ ID NO: 3 can be obtained from, for example, a tiger puffer (Takifugu rubripes).

In (Pt2) above, the “one or several” bases may be any number of bases as long as, for example, the protein encoded by the polynucleotide of (Pt2) above has the inosinic acid degrading activity. The “one or several” bases in (Pt2) above refer to, for example, 1 to 348 bases, 1 to 261 bases, 1 to 174 bases, 1 to 87 bases, 1 to 69 bases, 1 to 52 bases, 1 to 34 bases, 1 to 17 bases, 1 to 8 bases, 1 to 6 bases, 1 to 3 bases, 1 or 2 bases, or 1 base, in the base sequence of (Pt1) above.

In (Pt3) above, the “identity” may be any degree of identity as long as, for example, the protein encoded by the polynucleotide of (Pt3) above has the inosinic acid degrading activity.

In (Pt3) above, the identity to the base sequence of (Pt1) above is, for example, 80% or more, 85% or more, 90% or more, 95% or more, 96% or more, 97% or more, 98% or more, or 99% or more.

The “polynucleotide capable of hybridizing” in (Pt4) above is not limited as long as the protein encoded by the polynucleotide of (Pt4) above has the inosinic acid degrading activity. In (Pt4) above, the “polynucleotide capable of hybridizing” is, for example, a polynucleotide that is fully or partially complementary to the polynucleotide of (Pt1) above. The description in (Pn4) above can be applied to the hybridization and stringent condition in (Pt4) above.

The polynucleotide of (Pt5) above may have any base sequence as long as, for example, the protein encoded by the polynucleotide of (Pt5) above has the inosinic acid degrading activity. The base sequence of the polynucleotide of (Pt5) above can be designed through, for example, conversion to corresponding codons based on the amino acid sequence of SEQ ID NO: 4.

Amino Acid Sequence of NT5E Protein of Tiger Puffer (SEQ ID NO: 4)

MSVWSRRCALWVCISLLAGPVWTFELTLLHTNDNHARIEETSEDLGKCS
SRGPCFAGVARRFTKVSEIRKKEKNVLFLDAGDQFQGTVWFNYYKGAEA
AHFMNKLGYNAMALGNHEFDNGVEGLLPFLQNVNCSVVSANIQPDQSLA
AKLSGFLQPYTVLNVGSEKVAVVGYTTAETPFLSMPGPHLKFNEEVEAL
QVQVDKLETLGYDKIIALGHSGFDVDQQIAKRVRGVDVVIGGHTNTFLY
TGKAPSTEVPVGPYPFIVRSDDGRNVPVVQAFAFGKYLGYLRVTFDDAG
KVIKAAGNPILLDSSVPQDPDVLAEVNRWKKDLAQYSSQYVGQTLVYLN
GTFEECRFRECNLGNLICDGMIDHNIKFSSELQWNHVSLCMLNSGAIRA
PIDERYKNGSITMEDVLTVLPFGGTVDLVQIKGSTVKKAFEHAVHRFGS
MSGEFLQVSGFHVKYDLSKPVNQRVTSLSALCTECRVPKYEPIDPERTY
KVTMPSYLVDGGDGFSMIKEELLKHNTGDLDISVFSKYISQQKRVYPAV
EGRITVRGSASSAAHSLDVFLHTWPLLPYISPGSLKDKWL

In (Pt6) above, the “one or several” amino acids in the amino acid sequence may be any number of amino acids as long as, for example, the protein encoded by the polynucleotide of (Pt6) above has the inosinic acid degrading activity. The “one or several” amino acids in (Pt6) above refer to, for example, 1 to 115 amino acids, 1 to 86 amino acids, 1 to 57 amino acids, 1 to 28 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 4.

In (Pt7) above, the “identity” of the amino acid sequence may be any degree of identity as long as, for example, the protein encoded by the polynucleotide of (Pt7) above has the inosinic acid degrading activity. In (Pt7) above, the identity to the amino acid sequence of SEQ ID NO: 4 is, for example, 80% or more, 85% or more, 90% or more, 95% or more, 96% or more, 97% or more, 98% or more, or 99% or more.
(Po) Any of polynucleotides (Po1) to (Po7) below:
(Po1) a polynucleotide having a base sequence of SEQ ID NO: 5;
(Po2) a polynucleotide that encodes a protein having an activity of degrading inosinic acid and that has a base sequence consisting of the base sequence of (Po1) above with a deletion, a substitution, an insertion, and/or an addition of one or several bases;
(Po3) a polynucleotide that encodes a protein having an activity of degrading inosinic acid and that has a base sequence having 80% or more identity to the base sequence of (Po1) above;
(Po4) a polynucleotide that encodes a protein having an activity of degrading inosinic acid and that has a base sequence complementary to a polynucleotide capable of hybridizing to the polynucleotide having the base sequence of (Po1) above under a stringent condition;
(Po5) a polynucleotide encoding a protein having an amino acid sequence of SEQ ID NO: 6;
(Po6) a polynucleotide encoding a protein that has an activity of degrading inosinic acid and that has an amino acid sequence consisting of the amino acid sequence of SEQ ID NO: 6 with a deletion, a substitution, an insertion, and/or an addition of one or several amino acids; and
(Po7) a polynucleotide encoding a protein that has an activity of degrading inosinic acid and that has an amino acid sequence having 80% or more identity to the amino acid sequence of SEQ ID NO: 6.

Base Sequence of nt5e Gene of Tilapia (SEQ ID NO: 5)

5′-ATGTGCTATACATTCTTGGTCACAAATGCGTTTTTTACGCACGGAGACATCATGGGTTT
CCGTTCGTCTCGGCGCGCTCTCTTGACCTCGCTCTGCCTCGTTTTAAACTGCTGGAGC
GGAGCGTCGACCTTCGAGCTGACAATACTCCACACCAATGACAACCATGCGCGAATC
GAGGAGACCGGCAAGGACTCGGGAAAGTGTCGACCCGAGCGTCCCTGCTTCGCTGG
TGTGGCCAGGAGGTTCACGAAAGTGACCGAGATCAGAACAAAAGAGACGAACGTG
GTGTTTCTGGATGCTGGAGACCAATTCCAAGGGACACTCTGGTTCAACTACTATAAAG
GCGCTGAAGCCGCACACTTTATGAACAAACTTTGTTATGATGTTATGGCTTTTGGAAA
TCACGAGTTTGACAATGGAGTGGAGGGTCTCATTCACCCCTTCCTCCAAAATATAAAT
TTCTCTGTGGTCAGTGCAAACATTAAACCCGACTACACCCTGGCAGAGCTTCATAAAT
ACTACAGTCCCTACAAAGTTATCAGTGTGGGCTCAGAGAAAGTGGCTGTGGTTGGCT
ATACCTCAGCAGAGACGCCCTTCTTATCCATGCCAGGCAAACATCTCAAGTTCGAAGA
TGAGGTGGAGTCACTTCAAGCTCAAGTAAATAAGCTGGAAAGTCTGGGCTATAATAA
AATCATTGCCCTGGGACACTCTGGTTTTGTTGTGGATCAAGACATCGCAAGACGCGTG
AGAGGGGTGGACGTTGTTGTTGGAGGGCATACCAACACGTTCCTCTACACAGGAACG
CCCCCATCTTCCGAAGTGCCAGCTGGCCCATATCCTTTTATAGTAAAATCCAGCCATGG
AAGAGATGTGCCAGTGGTCCAGGCTTACGCCTTTGGGAAATATCTTGGACACTTAAA
AGTCACCTTTGATGATGCTGGCAATGTCATCAAAGCCGTTGGAAATCCCATCCTAATG
GACAGCAGCATACCTCAAGATGCAGAGATCCTTGCTGATGTTAATAAGTGGAAAACA
GACTTGGCTCAATACTCGACTAAGTATGTGGGCCAAACCTTAGTCTACCTCAACGGGT
CATTTGAAGAGTGCCGATTTCGGGAGTGTAACCTGGGAAACCTAATCTGTGATGCCAT
GGTGTATCACAATATGAGGCATTCAACTGGGGAGCAGTGGAATCATGTAAGCCTGTGT
ATGCTGAATAGTGGGGGCATACGGACAGCGATAGACGAACGCTACAGAAATGGCTCC
ATAACAATGGAGGAGATCCTCACCGTGTTACCATTTGGAGGAACCTGCGACTTGGTCC
AAATAAAAGGCTCAACAATAAAAAAGGCATTTGAACACTCTGTTCACAGATATGGAA
GCAAGACTGGAGAATTTCTTCAGGTGGTTTACGACCTCTCTAAGCCAGTGAACCAGC
GTGTAGCATCGCTGTCATTGCTCTGCACAGAGTGCCGAGTGCCCAAGTATGAACCATT
AGACCCACAGAAGACCTACACCGTGGTCATGCCTTCATACATGGTGGGCGGAGGCGA
TAACTTTACAATGATTAAGAGGGAGTTACTGAAACATAATTCAGGTGACTTGGACATT
ACCGTTTTTTCCAAGTACATCTCCGACATGAAGCGCGTGTTCCCTGCAGTAGAAGGCC
GCATCACTTTCAGGAACTCTGCTGTCATTGCTTCATACAGTATTGGCTTGTTGCTGCTG
AGTCTGTGTCTGTCTGTGACACTTTGA-3′

In (Po1) above, the base sequence of SEQ ID NO: 5 is the base sequence encoding the amino acid sequence of (Po5) above. The base sequence of SEQ ID NO: 5 can be obtained from, for example, a tilapia (Oreochromis niloticus).

In (Po2) above, the “one or several” bases may be any number of bases as long as, for example, the protein encoded by the polynucleotide of (Po2) above has the inosinic acid degrading activity. The “one or several” bases in (Po2) above refer to, for example, 1 to 351 bases, 1 to 263 bases, 1 to 175 bases, 1 to 87 bases, 1 to 70 bases, 1 to 52 bases, 1 to 35 bases, 1 to 17 bases, 1 to 8 bases, 1 to 6 bases, 1 to 3 bases, 1 or 2 bases, or 1 base, in the base sequence of (Po1) above.

In (Po3) above, the “identity” may be any degree of identity as long as, for example, the protein encoded by the polynucleotide of (Po3) above has the inosinic acid degrading activity. In (Po3) above, the identity to the base sequence of (Po1) above is, for example, 80% or more, 85% or more, 90% or more, 95% or more, 96% or more, 97% or more, 98% or more, or 99% or more.

The “polynucleotide capable of hybridizing” in (Po4) above is not limited as long as the protein encoded by the polynucleotide of (Po4) above has the inosinic acid degrading activity. In (Po4) above, the “polynucleotide capable of hybridizing” is, for example, a polynucleotide that is fully or partially complementary to the polynucleotide of (Po1) above. The description in (Pn4) above can be applied to the hybridization and stringent condition in (Po4) above.

The polynucleotide of (Po5) above may have any base sequence as long as, for example, the protein encoded by the polynucleotide of (Po5) above has the inosinic acid degrading activity. The base sequence of the polynucleotide of (Po5) above can be designed through, for example, conversion to corresponding codons based on the amino acid sequence of SEQ ID NO: 6.

Amino Acid Sequence of NTSE Protein of Tilapia (SEQ ID NO: 6)

MCYTFLVTNAFFTHGDIMGFRSSRRALLTSLCLVLNCWSGASTFELTIL
HTNDNHARIEETGKDSGKCRPERPCFAGVARRFTKVTEIRTKETNVVFL
DAGDQFQGTLWFNYYKGAEAAHFMNKLCYDVMAFGNHEFDNGVEGLIHP
FLQNINFSVVSANIKPDYTLAELHKYYSPYKVISVGSEKVAVVGYTSAE
TPFLSMPGKHLKFEDEVESLQAQVNKLESLGYNKIIALGHSGFVVDQDI
ARRVRGVDVVVGGHTNTFLYTGTPPSSEVPAGPYPFIVKSSHGRDVPVV
QAYAFGKYLGHLKVTFDDAGNVIKAVGNPILMDSSIPQDAEILADVNKW
KTDLAQYSTKYVGQTLVYLNGSFEECRFRECNLGNLICDAMVYHNMRHS
TGEQWNHVSLCMLNSGGIRTAIDERYRNGSITMEEILTVLPFGGTCDLV
QIKGSTIKKAFEHSVHRYGSKTGEFLQVVYDLSKPVNQRVASLSLLCTE
CRVPKYEPLDPQKTYTVVMPSYMVGGGDNFTMIKRELLKHNSGDLDITV
FSKYISDMKRVFPAVEGRITFRNSAVIASYSIGLLLLSLCLSVTL

In (Po6) above, the “one or several” amino acids in the amino acid sequence may be any number of amino acids as long as, for example, the protein encoded by the polynucleotide of (Po6) above has the inosinic acid degrading activity. The “one or several” amino acids in (Po6) above refer to, for example, 1 to 116 amino acids, 1 to 87 amino acids, 1 to 58 amino acids, 1 to 29 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 6.

In (Po7) above, the “identity” of the amino acid sequence may be any degree of identity as long as, for example, the protein encoded by the polynucleotide of (Po7) above has the inosinic acid degrading activity. In (Po7) above, the identity to the amino acid sequence of SEQ ID NO: 6 is, for example, 80% or more, 85% or more, 90% or more, 95% or more, 96% or more, 97% or more, 98% or more, or 99% or more.
(Pm) Any of polynucleotides (Pm1) to (Pm7) below:
(Pm1) a polynucleotide having a base sequence of SEQ ID NO: 7;
(Pm2) a polynucleotide that encodes a protein having an activity of degrading inosinic acid and that has a base sequence consisting of the base sequence of (Pm1) above with a deletion, a substitution, an insertion, and/or an addition of one or several bases;
(Pm3) a polynucleotide that encodes a protein having an activity of degrading inosinic acid and that has a base sequence having 80% or more identity to the base sequence of (Pm1) above;
(Pm4) a polynucleotide that encodes a protein having an activity of degrading inosinic acid and that has a base sequence complementary to a polynucleotide capable of hybridizing to the polynucleotide having the base sequence of (Pm1) above under a stringent condition;
(Pm5) a polynucleotide encoding a protein having an amino acid sequence of SEQ ID NO: 8;
(Pm6) a polynucleotide encoding a protein that has an activity of degrading inosinic acid and that has an amino acid sequence consisting of the amino acid sequence of SEQ ID NO: 8 with a deletion, a substitution, an insertion, and/or an addition of one or several amino acids; and
(Pm7) a polynucleotide encoding a protein that has an activity of degrading inosinic acid and that has an amino acid sequence having 80% or more identity to the amino acid sequence of SEQ ID NO: 8.

Base Sequence of nt5e Gene of Rainbow Trout (SEQ ID NO: 7)

5′-ATGAAAAAGGGTAACCAGCATATAATGTATGCGCCTAAAATTATTCCCAATGTTTTGGT
CGCTTTCCTTTCCCTAGCGGCTGAGATTCTTTTTTTCTCCAAAAGTGCGTCAGACACG
GTTACTTTCACTTGGTTCTCACTTGCCGAGGAACCCGTGTCACCGGAGTGGGTATCAT
GTCTTCCACACTGCAAAGGTGATGTCGATGCCACAGATCCAGGGGAGTTCGTGACCA
GGCGCGCTGAAGCATTTGGAAACCATGAGTTTGACAATGGAGTGGAGGGCCTGCTCC
GCCCTTTTCTGCAGAAGGTGAACTTCACTGTTCTCAGTGCCAACATCAAGGCAGATG
CAACACTCGCCCCAACTATCAATGGTTACTATCAACCCTACACAACATTCACGATGGG
CTCTGAAATAGTGGCTGTGGTAGGTTACACCTCAGTGGAAACTCCAGTCTTATCCTTA
CCAGGTCCACACCTGATCTTTGAGGATGAGATCAAAGCCTTGCAGGTCCAGGTGGAT
AAACTGATCACTCTTGGTTACAATAAGATCATCGCCCTTGGTCACTCTGGATTTGACG
TGGATATAGACATCGCCAAGCGAGTGAAAGGCGTGGACCTGGTCATCGGCGGACACA
CCAACACTTTCCTCTACACCGGGAGTGTCCCATCTTCAGAGGTGCCGGCGGGTCCCT
ACCCCTTCACGGTGAGGTCTGAGGACGGGAGAGATGTCCCTGTGGTACAGGCTTTTG
CCTTTGGGAAGTACCTGGGATACCTCAAGTTGGTCTTCGATAAATCTGGGAACGTGCT
GAAGGCGAATGGAAACCCCATCCTGCTGGACAGCAGCATAGCCCAGGATCCAGGTAT
CCTTGCTGATGTTGACGAGTGGAAGAAGAACTTGGCCCAGTACTCGTCTCAGTACGT
GGGGAAAACCCTGGTGTATCTCAATGGAACCTTCAATGAGTGCCGGTTCCGAGAGTG
CAACCTGGGGAACCTCATCTGTGACGCAATGATTCATCACAATATCAAGTATGCAGAT
GAGATCCAGTGGAATCACGTCAGTTTATGCATCCTGAACAGTGGAGGCATAAGGACA
GGAATAGATGAAAGTCACAAAAATGGCACCATCACCATGGAGGAAGTTATATCAGTC
TTACCATTTGGGGGCACTTTTGATCTGGTGCAGTTGAAGGGATCAACCTTGAAAAAG
GCATTTGAAAACTCTGTCCGAAGATACGGGAGCAGCAGAGGAGAATTTCTTCAAGTT
TCAGGAATCCATGTGGAATACGACCTGTCACGGTCGGTGGGGGACCGTGTGACGTCC
TTGAGCCTCCGCTGCTCTCAGTGCCGTGTGCCCAGATACGAATCTCTGGACCCAGAC
AGGCTGTACAAGCTGGTCCTACCATCCTACATCGCAGACGGAGGAGATGGTTTTACCA
TGATTAAGGAGGAGAAACTGAAACATGACACTGGTGATCTGGACATCTCGGTTTTCG
CCAACTACATCAAGGAGATGAAGAGGGTGTACCCAACTGTGGAGGGCCGCATCAAGT
TCAGAAACTCATCCGTGGCGGCTGGGGCCAACTGTCTGACTTTGTTACTGCTAGGCCT
GATGTGGGCCCTGTCCACAAGTTTATGA-3′

In (Pm1) above, the base sequence of SEQ ID NO: 7 is the base sequence encoding the amino acid sequence of (Pm5) above. The base sequence of SEQ ID NO: 7 can be obtained from, for example, a rainbow trout (Oncorhynchus mykiss).

In (Pm2) above, the “one or several” bases may be any number of bases as long as, for example, the protein encoded by the polynucleotide of (Pm2) above has the inosinic acid degrading activity. The “one or several” bases in (Pm2) above refer to, for example, 1 to 327 bases, 1 to 245 bases, 1 to 163 bases, 1 to 81 bases, 1 to 65 bases, 1 to 49 bases, 1 to 32 bases, 1 to 16 bases, 1 to 8 bases, 1 to 6 bases, 1 to 3 bases, 1 or 2 bases, or 1 base, in the base sequence of (Pm1) above.

In (Pm3) above, the “identity” may be any degree of identity as long as, for example, the protein encoded by the polynucleotide of (Pm3) above has the inosinic acid degrading activity. In (Pm3) above, the identity to the base sequence of (Pm1) above is, for example, 80% or more, 85% or more, 90% or more, 95% or more, 96% or more, 97% or more, 98% or more, or 99% or more.

The “polynucleotide capable of hybridizing” in (Pm4) above is not limited as long as the protein encoded by the polynucleotide of (Pm4) above has the inosinic acid degrading activity. In (Pm4) above, the “polynucleotide capable of hybridizing” is, for example, a polynucleotide that is fully or partially complementary to the polynucleotide of (Pm1) above. The description in (Pn4) above can be applied to the hybridization and stringent condition in (Pm4) above.

The polynucleotide of (Pm5) above may have any base sequence as long as, for example, the protein encoded by the polynucleotide of (Pm5) above has the inosinic acid degrading activity. The base sequence of the polynucleotide of (Pm5) above can be designed through, for example, conversion to corresponding codons based on the amino acid sequence of SEQ ID NO: 8.

Amino Acid Sequence of NT5E Protein of Rainbow Trout (SEQ ID NO: 8)

MKKGNQHIMYAPKIIPNVLVAFLSLAAEILFFSKSASDTVTFTWFSLAE
EPVSPEWVSCLPHCKGDVDATDPGEFVTRRAEAFGNHEFDNGVEGLLRP
FLQKVNFTVLSANIKADATLAPTINGYYQPYTTFTMGSEIVAVVGYTSV
ETPVLSLPGPHLIFEDEIKALQVQVDKLITLGYNKIIALGHSGFDVDID
IAKRVKGVDLVIGGHTNTFLYTGSVPSSEVPAGPYPFTVRSEDGRDVPV
VQAFAFGKYLGYLKLVFDKSGNVLKANGNPILLDSSIAQDPGILADVDE
WKKNLAQYSSQYVGKTLVYLNGTFNECRFRECNLGNLICDAMIHHNIKY
ADEIQWNHVSLCILNSGGIRTGIDESHKNGTITMEEVISVLPFGGTFDL
VQLKGSTLKKAFENSVRRYGSSRGEFLQVSGIHVEYDLSRSVGDRVTSL
SLRCSQCRVPRYESLDPDRLYKLVLPSYIADGGDGFTMIKEEKLKHDTG
DLDISVFANYIKEMKRVYPTVEGRIKFRNSSVAAGANCLTLLLLGLMWA
LSTSL

In (Pm6) above, the “one or several” amino acids in the amino acid sequence may be any number of amino acids as long as, for example, the protein encoded by the polynucleotide of (Pm6) above has the inosinic acid degrading activity. The “one or several” amino acids in (Pm6) above refer to, for example, 1 to 108 amino acids, 1 to 81 amino acids, 1 to 54 amino acids, 1 to 27 amino acids, 1 to 21 amino acids, 1 to 16 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 8.

In (Pm7) above, the “identity” of the amino acid sequence may be any degree of identity as long as, for example, the protein encoded by the polynucleotide of (Pm7) above has the inosinic acid degrading activity. In (Pm7) above, the identity to the amino acid sequence of SEQ ID NO: 8 is, for example, 80% or more, 85% or more, 90% or more, 95% or more, 96% or more, 97% or more, 98% or more, or 99% or more.
(Pg) Any of polynucleotides (Pg1) to (Pg7) below:
(Pg1) a polynucleotide having a base sequence of SEQ ID NO: 9;
(Pg2) a polynucleotide that encodes a protein having an activity of degrading inosinic acid and that has a base sequence consisting of the base sequence of (Pg1) above with a deletion, a substitution, an insertion, and/or an addition of one or several bases;
(Pg3) a polynucleotide that encodes a protein having an activity of degrading inosinic acid and that has a base sequence having 80% or more identity to the base sequence of (Pg1) above;
(Pg4) a polynucleotide that encodes a protein having an activity of degrading inosinic acid and that has a base sequence complementary to a polynucleotide capable of hybridizing to the polynucleotide having the base sequence of (Pg1) above under a stringent condition;
(Pg5) a polynucleotide encoding a protein having an amino acid sequence of SEQ ID NO: 10;
(Pg6) a polynucleotide encoding a protein that has an activity of degrading inosinic acid and that has an amino acid sequence consisting of the amino acid sequence of SEQ ID NO: 10 with a deletion, a substitution, an insertion, and/or an addition of one or several amino acids; and
(Pg7) a polynucleotide encoding a protein that has an activity of degrading inosinic acid and that has an amino acid sequence having 80% or more identity to the amino acid sequence of SEQ ID NO: 10.

Base Sequence of nt5e Gene of Honmoroko (SEQ ID NO: 9)

5′-ATGATGGCACAGTGGCTCAGCTCCATAAGCCTCCTCTGGATTCACTGTCAGCTGTGCA
GGACTGCGGAGTTCGAGCTGACTTTACTCCACACGAATGATGTCCACGCACGAGTGG
AGGAGACCAATAAAGACTCGGGGAAGTGCAGTAAAGCGCCTTGTTTCGCTGGAGTC
GCTCGGAGGTTGACGAAGATCAGGGAAGTCCGAGCCCAGGAGAAACACGTTCTGCT
GCTGGACGCGGGAGACCAGTTCCAGGGCACCGTCTGGTTTAACTTTTACAAAGGCGC
AGAAGCGGCGTATTTCATGAATAAACTCGGATACAATGCGATGGCCTTAGGAAATCAT
GAGTTTGACAATGGAGTGGACGGCCTCGTGAAACCTTTCCTTCAGGAAGTGAACTGC
TCTGTTCTGAGCGCAAATATTAAAGCCGATCAGACAATCGCTCCTCGGATCAGCGGCT
ACTATCTCCCCTATAAAATCTTCAATTTCACCTCAGAGAAAGTAGGTGTTGTTGGCTAC
ACATCCGTGGAGACCCCGGCTTTGTCTCTGCCAGGCCCCCATCTCCAGTTTGAGGATG
AGGTTACTGCTTTACAGCTTCAAGTGGACAAACTCACTGCTTTGGGTGTTAATAAGAT
CATTGCCCTTGGACATTCTGGCTTCCTAACGGACAAAAAGATCGCAAAGAAAGTTCG
GGGAGTGGATGTTGTGATTGGAGGGCACACCAATACATTCTTGTTCACAGGAGAGCC
ACCATCCACAGAGGTTCCCGCAGGACCGTACCCATTCATGGTGGATTCTGAGGATGG
CCGACAGGTTCCCGTGGTCCAGGCCTATGCATTTGGAAAATATCTGGGATTCCTAAAA
GTGACTTTTGATTCAAATGGGAATGTGGTGAAATCTTTTGGCAACCCAATTCTTCTGA
ATGGCTCAGTAGCACCAGATCCAGTCATTCAGGCTGAAGTGGACAACTGGAGGAAG
AATCTGGCCAATTACTCTTCTCAGTATGTAGGAGAGACTCTCGTCTATCTCAATGGGAC
TTTTGAGGAATGCCGATTTCGTGAATGTAATTTGGGAAATTTAATCTGTGATGCAATGG
TTCATAATAATATTAAATATGCTGATGAAATCCAGTGGAACCATGTCAGCTCTTGTATTC
TGAACGGTGGAGCCATTCGATCACCTATTGATGAGCGAAACAGAAATGGTTCCATCA
CGATGGAGGACCTGATCGCTGTCCTGCCGTTTGGAGGCACGTTTGACCTGGTCCAAA
TGAACGGATCTACTCTGCGAGAGGTCTTTGAGCACTCAGTTCGCAGATACGGAGGAA
ACACTGGAGAATTCCTACAGGTGTCAGGGTTTCAGCTGGTGTTTGATTTATCGAAACC
GCCAGGAAGCCGTGTTAAAAGTGTGAACGCGCTCTGCACGGAGTGTCGAGTGCCCC
GCTATGAACCGCTCATCCCCACGAAGGTGTATAAAGTGGTGCTGCCCTCTTACCTAGT
GGACGGAGGAGACGGATACACCATGATCAAAGAACAGAAACTCAAACATGACAGTG
GTGACCTGGATATAGCCGTAGTTGCCAGCTATATCACAGAGAGAAAGAGAGTTCATCC
AGCCGTGGAAGGACGCATTCAGTTTTCCAGCTCTTGCATTGCACATCGAGGATACAC
GGCCATCGTTCTGCTGGTGTGGGCGCTCTGGGTCATGATTGTTTAG-3′

In (Pg1) above, the base sequence of SEQ ID NO: 9 is the base sequence encoding the amino acid sequence of (Pg5) above. The base sequence of SEQ ID NO: 9 can be obtained from, for example, a honmoroko (Gnathopogon caerulescens).

In (Pg2) above, the “one or several” bases may be any number of bases as long as, for example, the protein encoded by the polynucleotide of (Pg2) above has the inosinic acid degrading activity. The “one or several” bases in (Pg2) above refer to, for example, 1 to 342 bases, 1 to 256 bases, 1 to 171 bases, 1 to 85 bases, 1 to 68 bases, 1 to 51 bases, 1 to 34 bases, 1 to 17 bases, 1 to 8 bases, 1 to 6 bases, 1 to 3 bases, 1 or 2 bases, or 1 base, in the base sequence of (Pg1) above.

In (Pg3) above, the “identity” may be any degree of identity as long as, for example, the protein encoded by the polynucleotide of (Pg3) above has the inosinic acid degrading activity. In (Pg3) above, the identity to the base sequence of (Pg1) above is, for example, 80% or more, 85% or more, 90% or more, 95% or more, 96% or more, 97% or more, 98% or more, or 99% or more.

The “polynucleotide capable of hybridizing” in (Pg4) above is not limited as long as the protein encoded by the polynucleotide of (Pg4) above has the inosinic acid degrading activity. In (Pg4) above, the “polynucleotide capable of hybridizing” is, for example, a polynucleotide that is fully or partially complementary to the polynucleotide of (Pg1) above. The description in (Pn4) above can be applied to the hybridization and stringent condition in (Pg4) above.

The polynucleotide of (Pg5) above may have any base sequence as long as, for example, the protein encoded by the polynucleotide of (Pg5) above has the inosinic acid degrading activity. The base sequence of the polynucleotide of (Pg5) above can be designed through, for example, conversion to corresponding codons based on the amino acid sequence of SEQ ID NO: 10.

Amino Acid Sequence of NT5E Protein of Honmoroko (SEQ ID NO: 10)

MMAQWLSSISLLWIHCQLCRTAEFELTLLHTNDVHARVEETNKDSGKCS
KAPCFAGVARRLTKIREVRAQEKHVLLLDAGDQFQGTVWFNFYKGAEAA
YFMNKLGYNAMALGNHEFDNGVDGLVKPFLQEVNCSVLSANIKADQTIA
PRISGYYLPYKIFNFTSEKVGVVGYTSVETPALSLPGPHLQFEDEVTAL
QLQVDKLTALGVNKIIALGHSGFLTDKKIAKKVRGVDVVIGGHTNTFLF
TGEPPSTEVPAGPYPFMVDSEDGRQVPVVQAYAFGKYLGFLKVTFDSNG
NVVKSFGNPILLNGSVAPDPVIQAEVDNWRKNLANYSSQYVGETLVYLN
GTFEECRFRECNLGNLICDAMVHNNIKYADEIQWNHVSSCILNGGAIRS
PIDERNRNGSITMEDLIAVLPFGGTFDLVQMNGSTLREVFEHSVRRYGG
NTGEFLQVSGFQLVFDLSKPPGSRVKSVNALCTECRVPRYEPLIPTKVY
KVVLPSYLVDGGDGYTMIKEQKLKHDSGDLDIAVVASYITERKRVHPAV
EGRIQFSSSCIAHRGYTAIVLLVWALWVMIV

In (Pg6) above, the “one or several” amino acids in the amino acid sequence may be any number of amino acids as long as, for example, the protein encoded by the polynucleotide of (Pg6) above has the inosinic acid degrading activity. The “one or several” amino acids in (Pg6) above refer to, for example, 1 to 114 amino acids, 1 to 85 amino acids, 1 to 57 amino acids, 1 to 28 amino acids, 1 to 22 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 10.

In (Pg7) above, the “identity” of the amino acid sequence may be any degree of identity as long as, for example, the protein encoded by the polynucleotide of (Pg7) above has the inosinic acid degrading activity. In (Pg7) above, the identity to the amino acid sequence of SEQ ID NO: 10 is, for example, 80% or more, 85% or more, 90% or more, 95% or more, 96% or more, 97% or more, 98% or more, or 99% or more.
(Pp) Any of polynucleotides (Pp1) to (Pp7) below:
(Pp1) a polynucleotide having a base sequence of SEQ ID NO: 11;
(Pp2) a polynucleotide that encodes a protein having an activity of degrading inosinic acid and that has a base sequence consisting of the base sequence of (Pp1) above with a deletion, a substitution, an insertion, and/or an addition of one or several bases;
(Pp3) a polynucleotide that encodes a protein having an activity of degrading inosinic acid and that has a base sequence having 80% or more identity to the base sequence of (Pp1) above;
(Pp4) a polynucleotide that encodes a protein having an activity of degrading inosinic acid and that has a base sequence complementary to a polynucleotide capable of hybridizing to the polynucleotide having the base sequence of (Pp1) above under a stringent condition;
(Pp5) a polynucleotide encoding a protein having an amino acid sequence of SEQ ID NO: 12;
(Pp6) a polynucleotide encoding a protein that has an activity of degrading inosinic acid and that has an amino acid sequence consisting of the amino acid sequence of SEQ ID NO: 12 with a deletion, a substitution, an insertion, and/or an addition of one or several amino acids; and
(Pp7) a polynucleotide encoding a protein that has an activity of degrading inosinic acid and that has an amino acid sequence having 80% or more identity to the amino acid sequence of SEQ ID NO: 12.

Base Sequence of nt5e Gene of Bastard Halibut (SEQ ID NO: 11)

5′-TCAGGACCTCACCTGAGGTTCGAGGACGAGGTGGACGCTCTGCAGCCACATGTGGA
CAAGCTGCGGACGCTGGGAGTGGATAAGATCATCGCTCTGGGTCACTCTGGTTTCAC
CAAGGACCAGGAGATCGCTAAGAAGGTCCGCGGAGTGGACGTGGTCATCGGAGGAC
ACACCAACACGTTCCTCTACACAGGAACTCCTCCCTCATCGGAAGTCCCCGCAGGTC
CGTATCCCTTTCTGGTGAAATCGGACGACGGGCGGCAGGTTCCCGTTGTGCAGGCCT
ACGCCTTCGGAAAATATCTGGGATATCTGAAAGTGACCTTTGACGATGCTGGAAACGT
GGAGAGGGCCACAGGAAACCCCATCCTGCTGAACAGCAGCTTCCCGCAGGACCCAG
ATGTTCTGGCTGATGTGGAGAAATGGAAGAAGAATCTGGCAAACTACTCGGCTCAGG
TTGTGGGACAGACTCTGGTCTTCCTGAACGGAGAAAGCGAGGAGTGTCGTTTCCGG
GAGTGTAACCTGGGAAACCTGATCTGTGACGCCATGGTCGACAACAACATCCGCATC
CCTGACGACGTCCAGTGGAACCACGTCAGCGCCAGCATCTTCAACGGAGGCGGCGT
CCGGGCGTCCATCGACGAGCAGTCCAGGAACGGTTCCATCACCATGGAGGACCTGAT
CTCCGTCTTACCGTTCGGAGGAACCTTCGACCTGGTGCAGCTGAGGGGCTCCACGCT
GAGGAAAGCGTTCGAACACTCAGTGAGACGATACGGACAGAGCACCGGAGAATTCC
TCCAAGTGTCCGGATTTCACGTAGAGTTCGATCTCTCCAAACCTCCAGGACGTCGTGT
GACGAGCCTCCGCATCCTCTGCACAGAGTGTCGCGTCCCTCACTACCAACCGGTGGA
GGACGAGACGGTTTACACGGTGGTGCTGACGTCCTACATGGTGAAGGGCGGGGACG
GATTCGACATGATCCAAAACGAGATTGTCAAGTACAACAGCGGAGATCTGGACATTT
CAGTCGTTTCCAGGTTCATCGGGAAGAGAAAGAAAGTTTATCCTCCTGTCGAAGGAC
GAATCAGGATCAAGAACTCTGCGTCGAGGCTGCAGGGACGAGCTGCTCTGCTGGTTT
CACTGTCGCTGCTCTGGAGTGTGTGCGGGACCATGTGA-3′

In (Pp1) above, the base sequence of SEQ ID NO: 11 is the base sequence encoding the amino acid sequence of (Pp5) above. The base sequence of SEQ ID NO: 11 can be obtained from, for example, a bastard halibut (Paralichthys olivaceus).

In (Pp2) above, the “one or several” bases may be any number of bases as long as, for example, the protein encoded by the polynucleotide of (Pp2) above has the inosinic acid degrading activity. The “one or several” bases in (Pp2) above refer to, for example, 1 to 234 bases, 1 to 175 bases, 1 to 117 bases, 1 to 58 bases, 1 to 47 bases, 1 to 35 bases, 1 to 23 bases, 1 to 11 bases, 1 to 8 bases, 1 to 6 bases, 1 to 3 bases, 1 or 2 bases, or 1 base, in the base sequence of (Pp l) above.

In (Pp3) above, the “identity” may be any degree of identity as long as, for example, the protein encoded by the polynucleotide of (Pp3) above has the inosinic acid degrading activity.

In (Pp3) above, the identity to the base sequence of (Pp l) above is, for example, 80% or more, 85% or more, 90% or more, 95% or more, 96% or more, 97% or more, 98% or more, or 99% or more.

The “polynucleotide capable of hybridizing” in (Pp4) above is not limited as long as the protein encoded by the polynucleotide of (Pp4) above has the inosinic acid degrading activity. In (Pp4) above, the “polynucleotide capable of hybridizing” is, for example, a polynucleotide that is fully or partially complementary to the polynucleotide of (Pp1) above. The description in (Pn4) above can be applied to the hybridization and stringent condition in (Pp4) above.

The polynucleotide of (Pp5) above may have any base sequence as long as, for example, the protein encoded by the polynucleotide of (Pp5) above has the inosinic acid degrading activity. The base sequence of the polynucleotide of (Pp5) above can be designed through, for example, conversion to corresponding codons based on the amino acid sequence of SEQ ID NO: 12.

Amino Acid Sequence of NT5E Protein of Bastard Halibut (SEQ ID NO: 12)

SGPHLRFEDEVDALQPHVDKLRTLGVDKIIALGHSGFTKDQEIAKKVRG
VDVVIGGHTNTFLYTGTPPSSEVPAGPYPFLVKSDDGRQVPVVQAYAFG
KYLGYLKVTFDDAGNVERATGNPILLNSSFPQDPDVLADVEKWKKNLAN
YSAQVVGQTLVFLNGESEECRFRECNLGNLICDAMVDNNIRIPDDVQWN
HVSASIFNGGGVRASIDEQSRNGSITMEDLISVLPFGGTFDLVQLRGST
LRKAFEHSVRRYGQSTGEFLQVSGFHVEFDLSKPPGRRVTSLRILCTEC
RVPHYQPVEDETVYTVVLTSYMVKGGDGFDMIQNEIVKYNSGDLDISVV
SRFIGKRKKVYPPVEGRIRIKNSASRLQGRAALLVSLSLLWSVCGTM

In (Pp6) above, the “one or several” amino acids in the amino acid sequence may be any number of amino acids as long as, for example, the protein encoded by the polynucleotide of (Pp6) above has the inosinic acid degrading activity. The “one or several” amino acids in (Pp6) above refer to, for example, 1 to 78 amino acids, 1 to 58 amino acids, 1 to 39 amino acids, 1 to 19 amino acids, 1 to 15 amino acids, 1 to 11 amino acids, 1 to 7 amino acids, 1 to 6 amino acids, 1 to 5 amino acids, 1 to 3 amino acids, 1 or 2 amino acids, or 1 amino acid, in the amino acid sequence of SEQ ID NO: 12.

In (Pp7) above, the “identity” of the amino acid sequence may be any degree of identity as long as, for example, the protein encoded by the polynucleotide of (Pp7) above has the inosinic acid degrading activity. In (Pp7) above, the identity to the amino acid sequence of SEQ ID NO: 12 is, for example, 80% or more, 85% or more, 90% or more, 95% or more, 96% or more, 97% or more, 98% or more, or 99% or more.
(Pq) Any of polynucleotides (Pq1) to (Pq7) below:
(Pq1) a polynucleotide having a base sequence of SEQ ID NO: 25;
(Pq2) a polynucleotide that encodes a protein having an activity of degrading inosinic acid and that has a base sequence consisting of the base sequence of (Pq1) above with a deletion, a substitution, an insertion, and/or an addition of one or several bases;
(Pq3) a polynucleotide that encodes a protein having an activity of degrading inosinic acid and that has a base sequence having 80% or more identity to the base sequence of (Pq1) above;
(Pq4) a polynucleotide that encodes a protein having an activity of degrading inosinic acid and that has a base sequence complementary to a polynucleotide capable of hybridizing to the polynucleotide having the base sequence of (Pq1) above under a stringent condition;
(Pq5) a polynucleotide encoding a protein having an amino acid sequence of SEQ ID NO: 26;
(Pq6) a polynucleotide encoding a protein that has an activity of degrading inosinic acid and that has an amino acid sequence consisting of the amino acid sequence of SEQ ID NO: 26 with a deletion, a substitution, an insertion, and/or an addition of one or several amino acids; and
(Pq7) a polynucleotide encoding a protein that has an activity of degrading inosinic acid and that has an amino acid sequence having 80% or more identity to the amino acid sequence of SEQ ID NO: 26.

Base Sequence of nt5e Gene of Whitespotted Clarias (SEQ ID NO: 25)

5′-ATGCGCTACTCGACAGCGGCGCGCGCGCTGCTCCCTTTGCTGGTAGTGTGCGCGAGC
TTGCGGCTCGCGGCGGCAGACTGGGAGCTGACGCTCTTGCACACCAACGACGTGCA
CGCGCGGGTCGAGGAGACCAACAAGGACTCGGGCAAGTGCACGAAGGGAGAGTGC
TTCGCCGGTGTCGCGCGGCGTTCCACGAAGATCAAAGAGATCCGGAGCAAGGAGAA
GAACGTGCTGCTGCTGGACGCAGGGGATCAGTTTCAGGGCACCGTCTGGTTCAACGT
CTACAAAGGCGATGAGGCGGCGCACTTCATGAACAAACTCCAATACGATGCCATGGC
TTTGGGAAACCACGAGTTTGACAATAGAGTGGAAGGTCTCATACCCTTCCTGCAGAA
GGTGAAGTGTCCTGTACTCAGCGCTAACATCAAGGCGGTTGAGCCTGTTGCGTCAAA
TATTAGCGGATATTTTTCACCTTATAAAATCTTAAATGTAACCTCTGAGAAAGTGGGTA
TAGTGGGATACACTACCAAGGAGACTCCAGCTCTGTCATTGCCAGGGCCGTACCTGC
ATTTTGAGGACGAGGTCGTAGCCGTGCAGCGGGAGGTAGACAAACTCATCGCTCTCG
GCGTTAACAAGATCATCGCCCTCGGCCATTCTGGATTTGACACGGACAAAGAGATCG
CCAAAAAGGTCCGTGGAGTGGACGTGGTGATTGGAGGACACACCAACACTTTTTTAT
ACACAGGGGCTCCCCCATCCAGCGAGGTTCCAGCAGGACCCTACCCATTAATGGTAC
AGTCCGATGATGGTCGCAAAGTGCCAGTGGTCCAGGCGTACGCCTTTGGGAAATACC
TTGGCTATCTCAAAGTGACTTTCGATTCTGCTGGAAACGTGATAAAAGCGGAAGGAA
ATCCAATCCTCCTAAATAGCAGCGTAACCGAAGATCCCAGCATTAAGGCAGATGTGGA
TACCTGGAAGGTCAAATTGGCCAATTACTCAGCTCAGTTTGTGGGAAACACTCTGGT
CTACCTGAACGGGACATTTGAAGAGTGTCGATTTCGGGAATGCAACCTGGGAAACTT
GATCTGCGATGCCATGGTCCATCACTACATCAAATATCCGGATGAACTCCAGTGGAAC
CATGTTAGTTCCTGTATTTTAAACGGAGGGGGCATACGATCCTCCATTGATGAGAGAA
GCAGAAATGGTAGCATCACTATGGAGGACGTGTTGACCGTGCTGCCGTTTGGAGGAA
CATATGATCTGGTTCAACTGAATGGCTCGACTCTTCTCCAGGCCTTCGAGCACAGCGT
CCACAGATATGGAGGAAACACTGGGGAGTTCCTACAGGTCTCAGGCTTTCAGCTGGA
GTACGACCTCACCAAGCCCTCTGGTCATCGTGTGATCAAGGCGAGAGTGTTATGTACC
GAGTGTCGAGTGCCACACTACGAGCCCCTGGACGCTACAAAAGTCTACAGGGTGGT
GATGCCATCCTACCTAGTGGACGGAGGAGACGGTTTCTCAATGATCAAGCAGCAGAT
CCTGAAACATGATAGTGGTGACCTCGACATATCCGTCTTTTCTCGCTACATCACAGAG
AGGCAGAGGGTTCACCCATCCGTGGAAGGACGAATCCGTCTCCTAAATTCTGCCGTG
GAAATCTCAAACTCTTCCTCCTACACCCTCTGCGTCGTGATTCTCATTATTGTGGGTGT
TATGCTCATTATGTTGGCTGGTGTCTACTACTACAGAAGGAAAGGTCTTGTTGATGATA
AGGTTACAGTACAGTACAGAAGAGCTATAGATGGAGATGAAAAACTGAATGACCAAA
CCACAGTAACGAGTTCCTGA-3′

In (Pq1) above, the base sequence of SEQ ID NO: 25 is the base sequence encoding the amino acid sequence of (Pq5) above. The base sequence of SEQ ID NO: 25 can be obtained from, for example, a whitespotted clarias (Clarias garienpinus).

In (Pq2) above, the “one or several” bases may be any number of bases as long as, for example, the protein encoded by the polynucleotide of (Pq2) above has the inosinic acid degrading activity. The “one or several” bases in (Pq2) above refer to, for example, 1 to 370 bases, 1 to 277 bases, 1 to 185 bases, 1 to 92 bases, 1 to 74 bases, 1 to 55 bases, 1 to 37 bases, 1 to 18 bases, 1 to 9 bases, 1 to 3 bases, 1 or 2 bases, or 1 base, in the base sequence of (Pq1) above.

In (Pq3) above, the “identity” may be any degree of identity as long as, for example, the protein encoded by the polynucleotide of (Pq3) above has the inosinic acid degrading activity. In (Pq3) above, the identity to the base sequence of (Pq1) above is, for example, 80% or more, 85% or more, 90% or more, 95% or more, 96% or more, 97% or more, 98% or more, or 99% or more.

The “polynucleotide capable of hybridizing” in (Pq4) above is not limited as long as the protein encoded by the polynucleotide of (Pq4) above has the inosinic acid degrading activity. In (Pq4) above, the “polynucleotide capable of hybridizing” is, for example, a polynucleotide that is fully or partially complementary to the polynucleotide of (Pq1) above. The description in (Pn4) above can be applied to the hybridization and stringent condition in (Pq4) above.

The polynucleotide of (Pq5) above may have any base sequence as long as, for example, the protein encoded by the polynucleotide of (Pq5) above has the inosinic acid degrading activity. The base sequence of the polynucleotide of (Pq5) above can be designed through, for example, conversion to corresponding codons based on the amino acid sequence of SEQ ID NO: 26.

Amino Acid Sequence of NT5E Protein of Whitespotted Clarias (SEQ ID NO: 26)

MRYSTAARALLPLLVVCASLRLAAADWELTLLHTNDVHARVEETNKDSG
KCTKGECFAGVARRSTKIKEIRSKEKNVLLLDAGDQFQGTVWFNVYKGD
EAAHFMNKLQYDAMALGNHEFDNRVEGLIPFLQKVKCPVLSANIKAVEP
VASNISGYFSPYKILNVTSEKVGIVGYTTKETPALSLPGPYLHFEDEVV
AVQREVDKLIALGVNKIIALGHSGFDTDKEIAKKVRGVDVVIGGHTNTF
LYTGAPPSSEVPAGPYPLMVQSDDGRKVPVVQAYAFGKYLGYLKVTFDS
AGNVIKAEGNPILLNSSVTEDPSIKADVDTWKVKLANYSAQFVGNTLVY
LNGTFEECRFRECNLGNLICDAMVHHYIKYPDELQWNHVSSCILNGGGI
RSSIDERSRNGSITMEDVLTVLPFGGTYDLVQLNGSTLLQAFEHSVHRY
GGNTGEFLQVSGFQLEYDLTKPSGHRVIKARVLCTECRVPHYEPLDATK
VYRVVMPSYLVDGGDGFSMIKQQILKHDSGDLDISVFSRYITERQRVHP
SVEGRIRLLNSAVEISNSSSYTLCVVILIIVGVMLIMLAGVYYYRRKGL
VDDKVTVQYRRAIDGDEKLNDQTTVTSS

In (Pq6) above, the “one or several” amino acids in the amino acid sequence may be any number of amino acids as long as, for example, the protein encoded by the polynucleotide of (Pq6) above has the inosinic acid degrading activity. The “one or several” amino acids in (Pq6) above refer to, for example, 1 to 123 amino acids, 1 to 92 amino acids, 1 to 61 amino acids, 1 to 30 amino acids, 1 to 24 amino acids, 1 to 18 amino acids, 1 to 12 amino acids, 1 to 6 amino acids, 1 to 3 amino acids, 1 or 2 amino acids, or 1 amino acid, in the amino acid sequence of SEQ ID NO: 26.

In (Pq7) above, the “identity” of the amino acid sequence may be any degree of identity as long as, for example, the protein encoded by the polynucleotide of (Pq7) above has the inosinic acid degrading activity. In (Pq7) above, the identity to the amino acid sequence of SEQ ID NO: 26 is, for example, 80% or more, 85% or more, 90% or more, 95% or more, 96% or more, 97% or more, 98% or more, or 99% or more.
(Pl) Any of polynucleotides (Pl1) to (Pl7) below:
(Pl1) a polynucleotide having a base sequence of SEQ ID NO: 13;
(Pl2) a polynucleotide that encodes a protein having an activity of degrading inosinic acid and that has a base sequence consisting of the base sequence of (Pl1) above with a deletion, a substitution, an insertion, and/or an addition of one or several bases;
(Pl3) a polynucleotide that encodes a protein having an activity of degrading inosinic acid and that has a base sequence having 80% or more identity to the base sequence of (Pl1) above;
(Pl4) a polynucleotide that encodes a protein having an activity of degrading inosinic acid and that has a base sequence complementary to a polynucleotide capable of hybridizing to the polynucleotide having the base sequence of (Pl1) above under a stringent condition;
(Pl5) a polynucleotide encoding a protein having an amino acid sequence of SEQ ID NO: 14;
(Pl6) a polynucleotide encoding a protein that has an activity of degrading inosinic acid and that has an amino acid sequence consisting of the amino acid sequence of SEQ ID NO: 14 with a deletion, a substitution, an insertion, and/or an addition of one or several amino acids; and
(Pl7) a polynucleotide encoding a protein that has an activity of degrading inosinic acid and that has an amino acid sequence having 80% or more identity to the amino acid sequence of SEQ ID NO: 14.

Base Sequence of nt5e Gene of Medaka (SEQ ID NO: 13)

5′-ATGACTCTCCGCTGGCGCTGCTGCGCCCTCGGCGCCTTGCTCGGTCTCCTTCTCCGGT
TAGACTCGTGGAGCGGAGCGTCCGGCTTCGAGCTGACTCTGCTCCACACCAACGACG
TCCACGCTCGCATCGAGGAGACCAGCGAGGACTCGTCCAAATGCCACGAAGCGGGC
TCGTGCTTCGCGGGGGTCGCCAGGATGTTCACCAAAGTGACGGAGATCCGGAGAAA
GGAGACGCACGTGCTGTTTCTGGACGCTGGAGATCAATTTCAAGGCACGGTGTGGTT
CAACTACTACAAAGGCAAAGAAGCTGCGCATTTCATGAACAAACTTGGTTATGACGT
CATGACCTTTGGGAACCACGAGTTTGATAATGGAGTGGACAGTCTCACGCAAAACTT
CCTCCAGCGCGTAAACTTCTCCGTAGTGTGTGCCACCATCAAACCGCTCCACAGTCTG
GTTGCAAACATGAGCCGCTTCTACAGACCCTTTGCTGTTTTTAACGTGGGCTCAGAGA
AGGTTGCTGTTGTTGGTTACACCACCAAAGAAACCCCTGTCTTATCTGCTCCAGGTCC
TTATCTGAAGTTTGAGGATGAGGTGGAAGCCCTCCAGGATCAGGTCAACCAGCTAGA
GAAGCTGGGTGTGAACAAGATCATCGCATTGGGACACTCTGGCTTTGAAGTGGACAA
AGACATCGCCAAGCGTGTGAGAGGAATCGATGTAGTGATCGGAGGACACACAAACA
CCTTCCTGTACACAGGAAAGCCTCCTTCTACCGAAGTGCCGAGAGGACCCTATCCTTT
CAATGTGAGCTCCAACGACGGGAGATGGGTGCCAGTGGTCCAAGCGTTCGCCTTCGG
AAAATACCTGGGATACCTGAAGGTGACGTTCGACCAAGCTGGGAAGGTCGTAAAAG
CCGTTGGAAACCCCATCCTGATGAACAGCAGCATCCCTCAAGATCCAGGGATCTTGTC
GGATGTTGAAAAGTGGAAGAAGGGCTTGGAGCAGTATTCTTCCCAGTACATTGGACA
AACTTTAGTCTACCTGAATGGAACGTTTGAAGAGTGTCGATTTAGGGAATGTAACCTG
GGGAACCTCATCTGTGATGCCATGATATACAACTATATTAGGTATTCCAACAAGCTGCA
GTGGAATCACGTGGGTGTTTGTATGCTGAACAGTGGATCCATACGAGCAACAATCGAT
GAACGCAGTACAAACGGCTCCATCACGATGGAGGAGATTCTCAGCGTCTTGCCGTTT
GGAGGAACTTTTGACTTAGTGCAGCTGAAAGGATCTACGCTAAAAAAGGCTTTTGAG
CACTCTGTTCACAGATATGGAGGGATGTCTGGAGAATTTCTTCAAGTGTCAGGTATCC
GTGTGGAGTACGACCTCTCCAAACCCGTCAACCAGCGCGTTGTATCGCTGTTAATGCG
TTGCACAGAATGCCGCGTTCCCAAGTTTGAACCGCTGGACCCTCAGAAGACGTACAC
CGTGGTCATGACCTCATTCATGGTGGGCGGAGGCGATGGCTATAGCATGATCCAAGAT
GAATTATTGAAGCATAACACAGGAAATTTGGACACTTTGGTTTTTTCCGAATACATCA
AGGACATGAGGCGCGTGTACCCTGCAGTGGAAGGCCGCATCACCTTCAGGAACTCTG
CAGCTTTTGCAGCTCACAGCCTGAGCCTGATGCTGCTGAGTCTGTGTCTTTTCCTGAA
CCTCTGCATGTGA-3′

In (Pl1) above, the base sequence of SEQ ID NO: 13 is the base sequence encoding the amino acid sequence of (Pl5) above. The base sequence of SEQ ID NO: 13 can be obtained from, for example, a medaka (Oryzias latipes).

In (Pl2) above, the “one or several” bases may be any number of bases as long as, for example, the protein encoded by the polynucleotide of (Pl2) above has the inosinic acid degrading activity. The “one or several” bases in (Pl2) above refer to, for example, 1 to 351 bases, 1 to 263 bases, 1 to 175 bases, 1 to 87 bases, 1 to 70 bases, 1 to 52 bases, 1 to 35 bases, 1 to 17 bases, 1 to 8 bases, 1 to 6 bases, 1 to 3 bases, 1 or 2 bases, or 1 base, in the base sequence of (Pl1) above.

In (Pl3) above, the “identity” may be any degree of identity as long as, for example, the protein encoded by the polynucleotide of (Pl3) above has the inosinic acid degrading activity. In (Pl3) above, the identity to the base sequence of (Pl1) above is, for example, 80% or more, 85% or more, 90% or more, 95% or more, 96% or more, 97% or more, 98% or more, or 99% or more.

The “polynucleotide capable of hybridizing” in (Pl4) above is not limited as long as the protein encoded by the polynucleotide of (Pl4) above has the inosinic acid degrading activity. In (Pl4) above, the “polynucleotide capable of hybridizing” is, for example, a polynucleotide that is fully or partially complementary to the polynucleotide of (Pl1) above. The description in (Pn4) above can be applied to the hybridization and stringent condition in (Pl4) above.

The polynucleotide of (Pl5) above may have any base sequence as long as, for example, the protein encoded by the polynucleotide of (Pl5) above has the inosinic acid degrading activity. The base sequence of the polynucleotide of (Pl5) above can be designed through, for example, conversion to corresponding codons based on the amino acid sequence of SEQ ID NO: 14.

Amino Acid Sequence of NTSE Protein of Medaka (SEQ ID NO: 14)

MTLRWRCCALGALLGLLLRLDSWSGASGFELTLLHTNDVHARIEETSED
SSKCHEAGSCFAGVARMFTKVTEIRRKETHVLFLDAGDQFQGTVWFNYY
KGKEAAHFMNKLGYDVMTFGNHEFDNGVDSLTQNFLQRVNFSVVCATIK
PLHSLVANMSRFYRPFAVFNVGSEKVAVVGYTTKETPVLSAPGPYLKFE
DEVEALQDQVNQLEKLGVNKIIALGHSGFEVDKDIAKRVRGIDVVIGGH
TNTFLYTGKPPSTEVPRGPYPFNVSSNDGRWVPVVQAFAFGKYLGYLKV
TFDQAGKVVKAVGNPILMNSSIPQDPGILSDVEKWKKGLEQYSSQYIGQ
TLVYLNGTFEECRFRECNLGNLICDAMIYNYIRYSNKLQWNHVGVCMLN
SGSIRATIDERSTNGSITMEEILSVLPFGGTFDLVQLKGSTLKKAFEHS
VHRYGGMSGEFLQVSGIRVEYDLSKPVNQRVVSLLMRCTECRVPKFEPL
DPQKTYTVVMTSFMVGGGDGYSMIQDELLKHNTGNLDTLVFSEYIKDMR
RVYPAVEGRITFRNSAAFAAHSLSLMLLSLCLFLNLCM

In (Pl6) above, the “one or several” amino acids in the amino acid sequence may be any number of amino acids as long as, for example, the protein encoded by the polynucleotide of (Pl6) above has the inosinic acid degrading activity. The “one or several” amino acids in (Pl6) above refer to, for example, 1 to 115 amino acids, 1 to 86 amino acids, 1 to 57 amino acids, 1 to 28 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 14.

In (Pl7) above, the “identity” of the amino acid sequence may be any degree of identity as long as, for example, the protein encoded by the polynucleotide of (Pl7) above has the inosinic acid degrading activity. In (Pl7) above, the identity to the amino acid sequence of SEQ ID NO: 14 is, for example, 80% or more, 85% or more, 90% or more, 95% or more, 96% or more, 97% or more, 98% or more, or 99% or more.

The “inosinic acid degrading activity” in the present disclosure may be evaluated, for example, based on the inosinic acid level in a biological sample from a target fish (test fish) or based on the expression of the nt5e gene or NT5E protein in a biological sample from a test fish. The biological sample from the test fish is not particularly limited, and may be, for example, the individual test fish or a portion thereof, and the skeletal muscle of the fish is preferable. For example, one type of biological sample or two or more types of biological samples may be used.

When evaluated based on the inosinic acid level in a biological sample from a test fish, the inosinic acid degrading activity can be evaluated based on the content of inosinic acid in fish meat one day (24 hours) after the death of the fish having the wild-type nt5e gene or loss-of-function nt5e gene. Specifically, the inosinic acid level in the biological sample is measured 24 hours after the death of the test fish in accordance with Example 1, which will be described later. When the inosinic acid level in the biological sample from the test fish is the same as (not significantly different from) that from a fish that is homozygous for the wild-type nt5e gene and/or is (significantly) lower than that from a fish that is homozygous for the wild-type nt5e gene and/or is (significantly) lower than that from a fish that is homozygous or heterozygous for the loss-of-function nt5e gene, the test fish can be, for example, determined to have the inosinic acid degrading activity. Meanwhile, when the inosinic acid level in the biological sample from the test fish is (significantly) higher than that from a fish that is homozygous for the wild-type nt5e gene and/or is the same as (not significantly different from) that from a fish that is homozygous or heterozygous for the loss-of-function nt5e gene and/or is (significantly) higher than that from a fish that is homozygous or heterozygous for the loss-of-function nt5e gene, the test fish can be, for example, determined to have no inosinic acid degrading activity.

When the evaluation is conducted based on the expression of the nt5e gene, the expression of the nt5e gene can be detected by, for example, detecting the expression of the mRNA of the nt5e gene. The mRNA can be extracted from the biological sample from the fish using a routine procedure. The expression of the mRNA of the nt5e gene can be detected through, for example, semi-quantitative PCR, quantitative PCR, northern blotting, digital PCR, RNA sequencing (RNAseq), or the like. Primers and/or probes used to detect the expression of the mRNA can be designed using, for example, common techniques in the art. When the nt5e gene expression level in the biological sample from the test fish is the same as (not significantly different from) that in a fish that is homozygous for the wild-type nt5e gene and/or is (significantly) higher than that in a fish that is homozygous for the wild-type nt5e gene and/or is (significantly) higher than that in the biological sample from a fish that is homozygous or heterozygous for the loss-of-function nt5e gene, the test fish can be, for example, determined to have the inosinic acid degrading activity. Meanwhile, when the nt5e gene expression level in the biological sample from the test fish is (significantly) lower than that from a fish that is homozygous for the wild-type nt5e gene and/or is the same as (not significantly different from) that from a fish that is homozygous or heterozygous for the loss-of-function nt5e gene and/or is (significantly) lower that from a fish that is homozygous or heterozygous for the loss-of-function nt5e gene, the test fish can be, for example, determined to have no inosinic acid degrading activity.

When the evaluation is conducted based on the expression of the NT5E protein, the expression of the NT5E protein can be detected using, for example, a method in which a spectrophotometer is used (e.g., UV absorption method or bicinchoninic acid method), ELISA, western blotting, or the like. The protein-containing extract from the fish can be prepared using common techniques in the art, such as ultrasonication and physical homogenization with a homogenizer. When the NT5E protein expression level in the biological sample from the test fish is the same as (not significantly different from) that in a fish that is homozygous for the wild-type NT5E protein and/or is (significantly) higher than that in a fish that is homozygous for the wild-type NT5E protein and/or is (significantly) higher than that in the biological sample from a fish that is homozygous or heterozygous for the loss-of-function NT5E protein, the test fish can be, for example, determined to have the inosinic acid degrading activity. Meanwhile, when the NT5E protein expression level in the biological sample from the test fish is (significantly) lower than that from a fish that is homozygous for the wild-type NT5E protein and/or is the same as (not significantly different from) that from a fish that is homozygous or heterozygous for the loss-of-function NT5E protein and/or is (significantly) lower than that from a fish that is homozygous or heterozygous for the loss-of-function NT5E protein, the test fish can be, for example, determined to have no inosinic acid degrading activity.

In the present disclosure, the nt5e gene can be present in an RNA form (e.g., mRNA) or DNA form (e.g., cDNA or genome DNA). The DNA may be a double-stranded DNA or single-stranded DNA. In the present disclosure, the gene may include an additional sequence such as an untranslated region (UTR).

The loss of the function of the nt5e gene means a state in which, in a fish having the loss-of-function nt5e gene, the function of the nt5e gene (significantly) decreases or is lost to an extent that the inosinic acid level in fish meat increases during ripening of the fish, compared to that of a fish having the wild-type nt5e gene (hereinafter also referred to as a “wild-type fish”). Specifically, the loss of the function of the nt5e gene may mean, for example, a state in which the expression level of the mRNA of the nt5e gene or the protein encoded by the gene (significantly) decreases, or the mRNA of the nt5e gene or the protein encoded by the gene is not fully expressed, or a state in which the expression level of the mRNA of a functional nt5e gene or the protein encoded by the nt5e gene decreases, or the mRNA of a functional nt5e gene or the protein encoded by the nt5e gene is not fully expressed. Accordingly, in the present disclosure, the loss of the function of the nt5e gene may be caused by introducing the loss-of-function mutation into the nt5e gene, or by introducing a polynucleotide for suppressing the expression of the nt5e gene. The term “suppressing the expression of the gene” may mean suppression of the transcription of the gene or suppression of the translation to a protein.

In the present disclosure, a fish in which the function of the nt5e gene is lost can also be referred to as, for example, a “fish having the loss-of-function nt5e gene”. A fish in which the function of the nt5e gene is lost may be, for example, heterozygous or homozygous for the loss-of-function nt5e gene. In the fish in which the function of the nt5e gene is lost, genes other than the nt5e gene may also be subjected to modification, alteration, introduction, and/or loss of the function.

The loss of the function of the nt5e gene can be caused by, for example, introducing a mutation, more specifically a loss-of-function mutation, into the nt5e gene. The type of mutation is not particularly limited, and examples thereof include a point mutation, a missense mutation, a nonsense mutation, a frameshift mutation, and a base deletion over a large area (large deletion). The mutation may cause, for example, partial or entire deletion of the nt5e gene. The frameshift mutation occurs when the triplet reading frames (codons) are shifted due to a base deletion or a base insertion. The frameshift mutation has a very high impact on the gene function compared to that of a base-pair substitution mutation. The reason for this is that, when the frameshift mutation occurs, the genetic codes on the downstream side of the position at which the frameshift mutation has been introduced are significantly shifted, which leads to not only changes in amino acids but also positional shifts of the stop codon and the like.

The loss-of-function nt5e gene is obtained by, for example, introducing a mutation such as an insertion, a deletion, and/or a substitution of one or several bases (hereinafter also referred to as “one or more bases”) into the base sequence of the wild-type nt5e gene. For example, the description of the number of bases in the descriptions of (Pn2), (Pt2), (Po2), (Pm2), (Pg2), (Pp2), (Pq2), and (Pl2) above can be applied to the “one or more bases”. The frameshift mutation is caused due to, for example, an insertion or a deletion of 3m+1 bases or 3m+2 bases (m is an integer larger than or equal to 0).

In the present disclosure, mutation in the nt5e gene can be caused by, for example, introducing a mutation into the target gene in the genome of a target fish using a routine procedure. A method for introducing the mutation can be a method such as homologous recombination or a genome-editing technology in which ZFN, TALEN, CRISPR-CAS9, CRISPR-CPF1, or the like is used. The method for introducing the mutation may be a mutagenesis such as a site-directed mutagenesis. The method for introducing the mutation may be, for example, a random mutagenesis. Examples of the random mutagenesis include: irradiation with α-rays, β-rays, γ-rays, X-rays, or the like; chemical treatment with a mutagenic agent such as ethyl methanesulfonate (EMS) or ethinylnitrosourea (ENU); and heavy ion beam treatment. The method for introducing the mutation with the genome editing technology can be conducted with reference to, for example, Example 1 below. Specifically, the introduction of the mutation with the genome editing technology can be conducted by, for example, introducing a protein and a nucleic acid included in the genome editing technology, or a vector that encodes the protein and the nucleic acid. The protein is, for example, a CRISPR (Clustered Regularly Interspaced Short Palindromic Repeats) enzyme, and specific examples thereof include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3 and Csf4. The above-mentioned nucleic acid may be crRNA and tracrRNA, or a single-stranded nucleic acid obtained by linking crRNA and tracrRNA via a linker. In this case, the nucleic acid is designed such that, for example, a base sequence of crRNA that anneals to the target sequence is complementary to the base sequence encoding the nt5e gene. One type of the nucleic acid may be used alone, or two or more types of the nucleic acids may be used together. When the genome editing technology is used, a large deletion in the base sequence can be induced between the target sequences using, for example, two or more types of nucleic acids. The method for introducing the mutation may be a mutagenesis such as a site-directed mutagenesis.

The position of the mutation in the loss-of-function nt5e gene, that is, the position at which the mutation is introduced into the nt5e gene, is not particularly limited, and can be located in any region related to the nt5e gene. Specific examples of the region in which the position of the mutation is located in the loss-of-function nt5e gene include the expression control region such as the promoter region for the nt5e gene, the exon region that includes the coding region encoding the protein encoded by the nt5e gene, and the non-coding region (e.g., an intron region, an enhancer region, or the like) that does not encode the protein encoded by the nt5e gene, and the exon region is preferable. The above-mentioned exon region is, for example, the first exon.

Specifically, when the fish is a red seabream, the position of the mutation in the nt5e gene may be located, for example, between the bases at positions 1 and 1200 and preferably between the bases at positions 1000 and 1100 or positions 1014 and 1036 in the base sequence of SEQ ID NO: 1. The above-mentioned position of the mutation in the nt5e gene corresponds to, for example, the sixth exon of the red seabream nt5e gene. When the fish is a tiger puffer, the position of the mutation in the nt5e gene may be located between the bases at positions 1 and 250 and preferably between the bases at positions 100 and 200 or positions 131 and 153 in the base sequence of SEQ ID NO: 3. The above-mentioned position of the mutation in the nt5e gene corresponds to, for example, the first exon of the tiger puffer nt5e gene. When the fish is a tilapia, the position of the mutation in the nt5e gene may be located between the bases at positions 1 and 300 and preferably between the bases at positions 200 and 250 or positions 217 and 239 in the base sequence of SEQ ID NO: 5. The above-mentioned position of the mutation in the nt5e gene corresponds to, for example, the first exon of the tilapia nt5e gene. When the fish is a bastard halibut, the position of the mutation in the nt5e gene may be located between the bases at positions 1 and 700 and preferably between the bases at positions 600 and 650 or positions 605 and 627 in the base sequence of SEQ ID NO: 5. The above-mentioned position of the mutation in the nt5e gene corresponds to, for example, the fourth exon of the bastard halibut nt5e gene. It is preferable to introduce the nonsense mutation or frameshift mutation at the position of the mutation.

When the fish is a red seabream, a tiger puffer, a tilapia, a rainbow trout, a honmoroko, a bastard halibut, a whitespotted clarias, or a medaka, examples of the loss-of-function nt5e gene of the fish include polynucleotides of (MN), (MT), (MO), (MM), (MG), (MP), (MQ), and (ML) below, or genome regions encoding these polynucleotides.
(MN) Any of polynucleotides (MN1) to (MN5) below;
(MN1) a polynucleotide having a base sequence consisting of the base sequence of SEQ ID NO: 1 with a deletion, a substitution, an insertion, and/or an addition of one or several bases;
(MN2) a polynucleotide having a base sequence having 80% or more identity to the base sequence of SEQ ID NO: 1;
(MN3) a polynucleotide having a base sequence complementary to a polynucleotide capable of hybridizing to the polynucleotide having the base sequence of SEQ ID NO: 1 under a stringent condition;
(MN4) a polynucleotide encoding a protein having an amino acid sequence consisting of the amino acid sequence of SEQ ID NO: 2 with a deletion, a substitution, an insertion, and/or an addition of one or several amino acids; and
(MN5) a polynucleotide encoding a protein having an amino acid sequence having 80% or more identity to the amino acid sequence of SEQ ID NO: 2.

The polynucleotides (MN1) to (MN5) above are preferably polynucleotides encoding proteins having no activity of degrading inosinic acid.

In the descriptions of the polynucleotides (MN1) to (MN5) above, the phrase “having no activity of degrading inosinic acid” means, for example, that the inosinic acid degrading activity is significantly suppressed as compared to that of the protein encoded by the polynucleotide (Pn1) or (Pn5) above, and preferably means that the inosinic acid degrading activity is completely lost.

The “one or several” bases in (MN1) above refer to, for example, 1 to 351 bases, 1 to 263 bases, 1 to 175 bases, 1 to 87 bases, 1 to 70 bases, 1 to 52 bases, 1 to 35 bases, 1 to 17 bases, 1 to 8 bases, 1 to 6 bases, 1 to 3 bases, 1 or 2 bases, or 1 base, in the base sequence of SEQ ID NO: 1.

In (MN2) above, the identity to the base sequence of SEQ ID NO: 1 is, for example, 80% or more, 85% or more, 90% or more, 95% or more, 96% or more, 97% or more, 98% or more, or 99% or more.

In (MN3) above, the “polynucleotide capable of hybridizing” is, for example, a polynucleotide that is fully or partially complementary to the polynucleotide having the base sequence of SEQ ID NO: 1. The hybridization can be detected, for example, through various hybridization assays. The description in (Pn4) above can be applied to the hybridization and stringent condition in (MN3) above.

The “one or several” amino acids in (MN4) above refer to, for example, 1 to 116 amino acids, 1 to 86 amino acids, 1 to 58 amino acids, 1 to 29 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 2.

In (MN5) above, the identity to the amino acid sequence of SEQ ID NO: 2 is, for example, 80% or more, 85% or more, 90% or more, 95% or more, 96% or more, 97% or more, 98% or more, or 99% or more.
(MT) Any of polynucleotides (MT1) to (MT5) below;
(MT1) a polynucleotide having a base sequence consisting of the base sequence of SEQ ID NO: 3 with a deletion, a substitution, an insertion, and/or an addition of one or several bases;
(MT2) a polynucleotide having a base sequence having 80% or more identity to the base sequence of SEQ ID NO: 3;
(MT3) a polynucleotide having a base sequence complementary to a polynucleotide capable of hybridizing to the polynucleotide having the base sequence of SEQ ID NO: 3 under a stringent condition;
(MT4) a polynucleotide encoding a protein having an amino acid sequence consisting of the amino acid sequence of SEQ ID NO: 4 with a deletion, a substitution, an insertion, and/or an addition of one or several amino acids; and
(MT5) a polynucleotide encoding a protein having an amino acid sequence having 80% or more identity to the amino acid sequence of SEQ ID NO: 4.

The polynucleotides (MT1) to (MT5) above are preferably polynucleotides encoding proteins having no activity of degrading inosinic acid.

In the descriptions of the polynucleotides (MT1) to (MT5) above, the phrase “having no activity of degrading inosinic acid” means, for example, that the inosinic acid degrading activity is significantly suppressed as compared to that of the protein encoded by the polynucleotide (Pt1) or (Pt5) above, and preferably means that the inosinic acid degrading activity is completely lost.

The “one or several” bases in (MT1) above refer to, for example, 1 to 348 bases, 1 to 261 bases, 1 to 174 bases, 1 to 87 bases, 1 to 69 bases, 1 to 52 bases, 1 to 34 bases, 1 to 17 bases, 1 to 8 bases, 1 to 6 bases, 1 to 3 bases, 1 or 2 bases, or 1 base, in the base sequence of SEQ ID NO: 3.

In (MT2) above, the identity to the base sequence of SEQ ID NO: 3 is, for example, 80% or more, 85% or more, 90% or more, 95% or more, 96% or more, 97% or more, 98% or more, or 99% or more.

In (MT3) above, the “polynucleotide capable of hybridizing” is, for example, a polynucleotide that is fully or partially complementary to the polynucleotide having the base sequence of SEQ ID NO: 3. The description in (Pn4) above can be applied to the hybridization and stringent condition in (MT3) above.

The “one or several” amino acids in (MT4) above refer to, for example, 1 to 115 amino acids, 1 to 86 amino acids, 1 to 57 amino acids, 1 to 28 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 4.

In (MT5) above, the identity to the amino acid sequence of SEQ ID NO: 4 is, for example, 80% or more, 85% or more, 90% or more, 95% or more, 96% or more, 97% or more, 98% or more, or 99% or more.
(MO) Any of polynucleotides (MO1) to (MO5) below;
(MO1) a polynucleotide having a base sequence consisting of the base sequence of SEQ ID NO: 5 with a deletion, a substitution, an insertion, and/or an addition of one or several bases;
(MO2) a polynucleotide having a base sequence having 80% or more identity to the base sequence of SEQ ID NO: 5;
(MO3) a polynucleotide having a base sequence complementary to a polynucleotide capable of hybridizing to the polynucleotide having the base sequence of SEQ ID NO: 5 under a stringent condition;
(MO4) a polynucleotide encoding a protein having an amino acid sequence consisting of the amino acid sequence of SEQ ID NO: 6 with a deletion, a substitution, an insertion, and/or an addition of one or several amino acids; and
(MO5) a polynucleotide encoding a protein having an amino acid sequence having 80% or more identity to the amino acid sequence of SEQ ID NO: 6.

The polynucleotides (MO1) to (MO5) above are preferably polynucleotides encoding proteins having no inosinic acid degrading activity.

In the descriptions of the polynucleotides (MO1) to (MO5) above, the phrase “having no activity of degrading inosinic acid” means, for example, that the inosinic acid degrading activity is significantly suppressed as compared to that of the protein encoded by the polynucleotide (Po1) or (Po5) above, and preferably means that the inosinic acid degrading activity is completely lost.

The “one or several” bases in (MO1) above refer to, for example, 1 to 351 bases, 1 to 263 bases, 1 to 175 bases, 1 to 87 bases, 1 to 70 bases, 1 to 52 bases, 1 to 35 bases, 1 to 17 bases, 1 to 8 bases, 1 to 6 bases, 1 to 3 bases, 1 or 2 bases, or 1 base, in the base sequence of SEQ ID NO: 5.

In (MO2) above, the identity to the base sequence of SEQ ID NO: 5 is, for example, 80% or more, 85% or more, 90% or more, 95% or more, 96% or more, 97% or more, 98% or more, or 99% or more.

In (MO3) above, the “polynucleotide capable of hybridizing” is, for example, a polynucleotide that is fully or partially complementary to the polynucleotide having the base sequence of SEQ ID NO: 5. The description in (Pn4) above can be applied to the hybridization and stringent condition in (MO3) above.

The “one or several” amino acids in (MO4) above refer to, for example, 1 to 116 amino acids, 1 to 87 amino acids, 1 to 58 amino acids, 1 to 29 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 6.

In (MO5) above, the identity to the amino acid sequence of SEQ ID NO: 6 is, for example, 80% or more, 85% or more, 90% or more, 95% or more, 96% or more, 97% or more, 98% or more, or 99% or more.
(MM) Any of polynucleotides (M/I) to (MM5) below;
(MM1) a polynucleotide having a base sequence consisting of the base sequence of SEQ ID NO: 7 with a deletion, a substitution, an insertion, and/or an addition of one or several bases;
(MM2) a polynucleotide having a base sequence having 80% or more identity to the base sequence of SEQ ID NO: 7;
(MM3) a polynucleotide having a base sequence complementary to a polynucleotide capable of hybridizing to the polynucleotide having the base sequence of SEQ ID NO: 7 under a stringent condition;
(MM4) a polynucleotide encoding a protein having an amino acid sequence consisting of the amino acid sequence of SEQ ID NO: 8 with a deletion, a substitution, an insertion, and/or an addition of one or several amino acids; and
(MM5) a polynucleotide encoding a protein having an amino acid sequence having 80% or more identity to the amino acid sequence of SEQ ID NO: 8.

The polynucleotides (M I) to (MM5) above are preferably polynucleotides encoding proteins having no activity of degrading inosinic acid.

In the descriptions of the polynucleotides (MM1) to (MM5) above, the phrase “having no activity of degrading inosinic acid” means, for example, that the inosinic acid degrading activity is significantly suppressed as compared to that of the protein encoded by the polynucleotide (Pm1) or (Pm5) above, and preferably means that the inosinic acid degrading activity is completely lost.

The “one or several” bases in (MM1) above refer to, for example, 1 to 327 bases, 1 to 245 bases, 1 to 163 bases, 1 to 81 bases, 1 to 65 bases, 1 to 49 bases, 1 to 32 bases, 1 to 16 bases, 1 to 8 bases, 1 to 6 bases, 1 to 3 bases, 1 or 2 bases, or 1 base, in the base sequence of SEQ ID NO: 7.

In (MM2) above, the identity to the base sequence of SEQ ID NO: 7 is, for example, 80% or more, 85% or more, 90% or more, 95% or more, 96% or more, 97% or more, 98% or more, or 99% or more.

In (MM3) above, the “polynucleotide capable of hybridizing” is, for example, a polynucleotide that is fully or partially complementary to the polynucleotide having the base sequence of SEQ ID NO: 7. The hybridization can be detected, for example, through various hybridization assays. The description in (Pn4) above can be applied to the hybridization and stringent condition in (MM3) above.

The “one or several” amino acids in (MM4) above refer to, for example, 1 to 108 amino acids, 1 to 81 amino acids, 1 to 54 amino acids, 1 to 27 amino acids, 1 to 21 amino acids, 1 to 16 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 8.

In (MM5) above, the identity to the amino acid sequence of SEQ ID NO: 8 is, for example, 80% or more, 85% or more, 90% or more, 95% or more, 96% or more, 97% or more, 98% or more, or 99% or more.
(MG) Any of polynucleotides (MG1) to (MG5) below;
(MG1) a polynucleotide having a base sequence consisting of the base sequence of SEQ ID NO: 9 with a deletion, a substitution, an insertion, and/or an addition of one or several bases;
(MG2) a polynucleotide having a base sequence having 80% or more identity to the base sequence of SEQ ID NO: 9;
(MG3) a polynucleotide having a base sequence complementary to a polynucleotide capable of hybridizing to the polynucleotide having the base sequence of SEQ ID NO: 9 under a stringent condition;
(MG4) a polynucleotide encoding a protein having an amino acid sequence consisting of the amino acid sequence of SEQ ID NO: 10 with a deletion, a substitution, an insertion, and/or an addition of one or several amino acids; and
(MG5) a polynucleotide encoding a protein having an amino acid sequence having 80% or more identity to the amino acid sequence of SEQ ID NO: 10.

The polynucleotides (MG1) to (MG5) above are preferably polynucleotides encoding proteins having no activity of degrading inosinic acid.

In the descriptions of the polynucleotides (MG1) to (MG5) above, the phrase “having no activity of degrading inosinic acid” means, for example, that the inosinic acid degrading activity is significantly suppressed as compared to that of the protein encoded by the polynucleotide (Pg1) or (Pg5) above, and preferably means that the inosinic acid degrading activity is completely lost.

The “one or several” bases in (MG1) above refer to, for example, 1 to 327 bases, 1 to 245 bases, 1 to 163 bases, 1 to 81 bases, 1 to 65 bases, 1 to 49 bases, 1 to 32 bases, 1 to 16 bases, 1 to 8 bases, 1 to 6 bases, 1 to 3 bases, 1 or 2 bases, or 1 base, in the base sequence of SEQ ID NO: 9.

In (MG2) above, the identity to the base sequence of SEQ ID NO: 9 is, for example, 80% or more, 85% or more, 90% or more, 95% or more, 96% or more, 97% or more, 98% or more, or 99% or more.

In (MG3) above, the “polynucleotide capable of hybridizing” is, for example, a polynucleotide that is fully or partially complementary to the polynucleotide having the base sequence of SEQ ID NO: 9. The hybridization can be detected, for example, through various hybridization assays. The description in (Pn4) above can be applied to the hybridization and stringent condition in (MG3) above.

The “one or several” amino acids in (MG4) above refer to, for example, 1 to 108 amino acids, 1 to 81 amino acids, 1 to 54 amino acids, 1 to 27 amino acids, 1 to 21 amino acids, 1 to 16 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 10.

In (MG5) above, the identity to the amino acid sequence of SEQ ID NO: 10 is, for example, 80% or more, 85% or more, 90% or more, 95% or more, 96% or more, 97% or more, 98% or more, or 99% or more.
(MP) Any of polynucleotides (MP1) to (MP5) below;
(MP1) a polynucleotide having a base sequence consisting of the base sequence of SEQ ID NO: 11 with a deletion, a substitution, an insertion, and/or an addition of one or several bases;
(MP2) a polynucleotide having a base sequence having 80% or more identity to the base sequence of SEQ ID NO: 11;
(MP3) a polynucleotide having a base sequence complementary to a polynucleotide capable of hybridizing to the polynucleotide having the base sequence of SEQ ID NO: 11 under a stringent condition;
(MP4) a polynucleotide encoding a protein having an amino acid sequence consisting of the amino acid sequence of SEQ ID NO: 12 with a deletion, a substitution, an insertion, and/or an addition of one or several amino acids; and
(MP5) a polynucleotide encoding a protein having an amino acid sequence having 80% or more identity to the amino acid sequence of SEQ ID NO: 12.

The polynucleotides (MP1) to (MP5) above are preferably polynucleotides encoding proteins having no activity of degrading inosinic acid.

In the descriptions of the polynucleotides (MP1) to (MP5) above, the phrase “having no activity of degrading inosinic acid” means, for example, that the inosinic acid degrading activity is significantly suppressed as compared to that of the protein encoded by the polynucleotide (Pg1) or (Pg5) above, and preferably means that the inosinic acid degrading activity is completely lost.

The “one or several” bases in (MPi) above refer to, for example, 1 to 327 bases, 1 to 245 bases, 1 to 163 bases, 1 to 81 bases, 1 to 65 bases, 1 to 49 bases, 1 to 32 bases, 1 to 16 bases, 1 to 8 bases, 1 to 6 bases, 1 to 3 bases, 1 or 2 bases, or 1 base, in the base sequence of SEQ ID NO: 11.

In (MP2) above, the identity to the base sequence of SEQ ID NO: 11 is, for example, 80% or more, 85% or more, 90% or more, 95% or more, 96% or more, 97% or more, 98% or more, or 99% or more.

In (MP3) above, the “polynucleotide capable of hybridizing” is, for example, a polynucleotide that is fully or partially complementary to the polynucleotide having the base sequence of SEQ ID NO: 11. The hybridization can be detected, for example, through various hybridization assays. The description in (Pn4) above can be applied to the hybridization and stringent condition in (MP3) above.

The “one or several” amino acids in (MP4) above refer to, for example, 1 to 108 amino acids, 1 to 81 amino acids, 1 to 54 amino acids, 1 to 27 amino acids, 1 to 21 amino acids, 1 to 16 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 12.

In (MP5) above, the identity to the amino acid sequence of SEQ ID NO: 12 is, for example, 80% or more, 85% or more, 90% or more, 95% or more, 96% or more, 97% or more, 98% or more, or 99% or more.
(MQ) Any of polynucleotides (MQ1) to (MQ5) below;
(MQ1) a polynucleotide having a base sequence consisting of the base sequence of SEQ ID NO: 25 with a deletion, a substitution, an insertion, and/or an addition of one or several bases;
(MQ2) a polynucleotide having a base sequence having 80% or more identity to the base sequence of SEQ ID NO: 25;
(MQ3) a polynucleotide having a base sequence complementary to a polynucleotide capable of hybridizing to the polynucleotide having the base sequence of SEQ ID NO: 25 under a stringent condition;
(MQ4) a polynucleotide encoding a protein having an amino acid sequence consisting of the amino acid sequence of SEQ ID NO: 25 with a deletion, a substitution, an insertion, and/or an addition of one or several amino acids; and
(MQ5) a polynucleotide encoding a protein having an amino acid sequence having 80% or more identity to the amino acid sequence of SEQ ID NO: 26.

The polynucleotides (MQ1) to (MQ5) above are preferably polynucleotides encoding proteins having no activity of degrading inosinic acid.

In the descriptions of the polynucleotides (MQ1) to (MQ5) above, the phrase “having no activity of degrading inosinic acid” means, for example, that the inosinic acid degrading activity is significantly suppressed as compared to that of the protein encoded by the polynucleotide (Pq1) or (Pq5) above, and preferably means that the inosinic acid degrading activity is completely lost.

The “one or several” bases in (MQ1) above refer to, for example, 1 to 370 bases, 1 to 277 bases, 1 to 185 bases, 1 to 92 bases, 1 to 74 bases, 1 to 55 bases, 1 to 37 bases, 1 to 18 bases, 1 to 9 bases, 1 to 3 bases, 1 or 2 bases, or 1 base, in the base sequence of SEQ ID NO: 25.

In (MQ2) above, the identity to the base sequence of SEQ ID NO: 25 is, for example, 80% or more, 85% or more, 90% or more, 95% or more, 96% or more, 97% or more, 98% or more, or 99% or more.

In (MQ3) above, the “polynucleotide capable of hybridizing” is, for example, a polynucleotide that is fully or partially complementary to the polynucleotide having the base sequence of SEQ ID NO: 25. The description in (Pn4) above can be applied to the hybridization and stringent condition in (MQ3) above.

The “one or several” amino acids in (MQ4) above refer to, for example, 1 to 123 amino acids, 1 to 92 amino acids, 1 to 61 amino acids, 1 to 30 amino acids, 1 to 24 amino acids, 1 to 18 amino acids, 1 to 12 amino acids, 1 to 6 amino acids, 1 to 3 amino acids, 1 or 2 amino acids, or 1 amino acid, in the amino acid sequence of SEQ ID NO: 26.

In (MQ5) above, the identity to the amino acid sequence of SEQ ID NO: 26 is, for example, 80% or more, 85% or more, 90% or more, 95% or more, 96% or more, 97% or more, 98% or more, or 99% or more.
(ML) Any of polynucleotides (ML1) to (ML5) below;
(ML1) a polynucleotide having a base sequence consisting of the base sequence of SEQ ID NO: 13 with a deletion, a substitution, an insertion, and/or an addition of one or several bases;
(ML2) a polynucleotide having a base sequence having 80% or more identity to the base sequence of SEQ ID NO: 13;
(ML3) a polynucleotide having a base sequence complementary to a polynucleotide capable of hybridizing to the polynucleotide having the base sequence of SEQ ID NO: 13 under a stringent condition;
(ML4) a polynucleotide encoding a protein having an amino acid sequence consisting of the amino acid sequence of SEQ ID NO: 14 with a deletion, a substitution, an insertion, and/or an addition of one or several amino acids; and
(ML5) a polynucleotide encoding a protein having an amino acid sequence having 80% or more identity to the amino acid sequence of SEQ ID NO: 14.

The polynucleotides (ML1) to (ML5) above are preferably polynucleotides encoding proteins having no inosinic acid degrading activity.

In the descriptions of the polynucleotides (ML1) to (ML5) above, the phrase “having no inosinic acid degrading activity” means, for example, that the inosinic acid degrading activity is significantly suppressed as compared to that of the protein encoded by the polynucleotide (Pl1) or (Pl5) above, and preferably means that the inosinic acid degrading activity is completely lost.

The “one or several” bases in (ML1) above refer to, for example, 1 to 351 bases, 1 to 263 bases, 1 to 175 bases, 1 to 87 bases, 1 to 70 bases, 1 to 52 bases, 1 to 35 bases, 1 to 17 bases, 1 to 8 bases, 1 to 6 bases, 1 to 3 bases, 1 or 2 bases, or 1 base, in the base sequence of SEQ ID NO: 13.

In (MHL2) above, the identity to the base sequence of SEQ ID NO: 13 is, for example, 80% or more, 85% or more, 90% or more, 95% or more, 96% or more, 97% or more, 98% or more, or 99% or more.

In (ML3) above, the “polynucleotide capable of hybridizing” is, for example, a polynucleotide that is fully or partially complementary to the polynucleotide having the base sequence of SEQ ID NO: 13. The description in (Pn) above can be applied to the hybridization and stringent condition in (ML3) above.

The “one or several” amino acids in (ML4) above refer to, for example, 1 to 115 amino acids, 1 to 86 amino acids, 1 to 57 amino acids, 1 to 28 amino acids, 1 to 23 amino acids, 1 to 17 amino acids, 1 to 11 amino acids, 1 to 8 amino acids, 1 to 6 amino acids, 1 to 5 amino acids, 1 to 3 amino acids, 1 or 2 amino acids, or 1 amino acid, in the amino acid sequence of SEQ ID NO: 14.

In (MHL5) above, the identity to the amino acid sequence of SEQ ID NO: 14 is, for example, 80% or more, 85% or more, 90% or more, 95% or more, 96% or more, 97% or more, 98% or more, or 99% or more.

When the fish is a medaka, another example of the loss-of-function nt5e gene of a fish is a polynucleotide (Ml) below or a genome region encoding this polynucleotide.
(Ml) Any of polynucleotides (Ml1) to (M17) below:
(Ml1) a polynucleotide having a base sequence of SEQ ID NO: 15; (M12) a polynucleotide that encodes a protein having no activity of degrading inosinic acid and that has a base sequence consisting of the base sequence of (Ml1) above with a deletion, a substitution, an insertion, and/or an addition of one or several bases;
(Ml3) a polynucleotide that encodes a protein having no activity of degrading inosinic acid and that has a base sequence having 80% or more identity to the base sequence of (Ml1) above;
(Ml4) a polynucleotide that encodes a protein having no activity of degrading inosinic acid and that has a base sequence complementary to a polynucleotide capable of hybridizing to the polynucleotide having the base sequence of (Ml1) above under a stringent condition;
(Ml5) a polynucleotide encoding a protein having an amino acid sequence of SEQ ID NO: 16;
(Ml6) a polynucleotide encoding a protein that has no activity of degrading inosinic acid and that has an amino acid sequence consisting of the amino acid sequence of SEQ ID NO: 16 with a deletion, a substitution, an insertion, and/or an addition of one or several amino acids; and
(Ml7) a polynucleotide encoding a protein that has no activity of degrading inosinic acid and that has an amino acid sequence having 80% or more identity to the amino acid sequence of SEQ ID NO: 16.

In the descriptions of the polynucleotides (Ml1) to (Ml7) above, the phrase “having no inosinic acid degrading activity” means, for example, that the inosinic acid degrading activity is significantly suppressed as compared to that of the protein encoded by the polynucleotide (Pl1) or (Pl5) above, and preferably means that the inosinic acid degrading activity is completely lost.

Base Sequence of Loss-of-Function nt5e Gene of Medaka (SEQ ID NO: 15)

5′-ATGACTCTCCGCTGGCGCTGCTGCGCCCTCGGCGCCTTGCTCGGTC
TCCTTCTCCGGTTAGACTCGTGGAGCGGAGCGTCCGGCTTCGAGCTGAC
TCTGCTCCACACCAACGACGTCCACGCTCGCATCGAGGAGACCAGCGAG
GACTCGTCCAAATGCCACGAAGCGGGCTCGTGCTTCGCGGGGGTCGCCA
GGATGTTCACCAAAGACGGAGATCCGGAGAAAGGAGACGCACGTGCTGT
TTCTGGACGCTGGAGATCAATTTCAAGGCACGGTGTGGTTCAACTACTA
CAAAGGCAAAGAAGCTGCGCATTTCATGAACAAACTTGGTTATGA-3′

In (Ml1) above, the base sequence of SEQ ID NO: 15 above is a base sequence consisting of the base sequence of SEQ ID NO: 13 with deletion of the bases at positions 208 and 209 and the bases between positions 339 and 1734. The base sequence of SEQ ID NO: 15 above is the base sequence encoding the amino acid sequence of (Ml5).

In (Ml2) above, the “one or several” bases may be any number of bases as long as, for example, the protein encoded by the polynucleotide of (Ml2) above has no inosinic acid degrading activity. The “one or several” bases in (Ml2) above refer to, for example, 1 to 67 bases, 1 to 50 bases, 1 to 33 bases, 1 to 16 bases, 1 to 13 bases, 1 to 10 bases, 1 to 6 bases, 1 to 3 bases, 1 or 2 bases, or 1 base, in the base sequence of (Ml1) above.

In (Ml3) above, the “identity” may be any degree of identity as long as, for example, the protein encoded by the polynucleotide of (Ml3) above has no inosinic acid degrading activity. In (Ml3) above, the identity to the base sequence of (Ml1) above is, for example, 80% or more, 85% or more, 90% or more, 95% or more, 96% or more, 97% or more, 98% or more, or 99% or more.

The “polynucleotide capable of hybridizing” in (Ml4) above is not limited as long as the protein encoded by the polynucleotide of (Ml4) above has no inosinic acid degrading activity. In (Ml4) above, the “polynucleotide capable of hybridizing” is, for example, a polynucleotide that is fully or partially complementary to the polynucleotide of (Ml1) above. The description in (Pn4) above can be applied to the hybridization and stringent condition in (Ml4) above.

The polynucleotide of (Ml5) above may have any base sequence as long as, for example, the protein encoded by the polynucleotide of (Ml5) above has no inosinic acid degrading activity. The base sequence of the polynucleotide of (Ml5) above can be designed through, for example, conversion to corresponding codons based on the amino acid sequence of SEQ ID NO: 16.

Amino Acid Sequence of Mutant NT5E Protein of Medaka (SEQ ID NO: 16)

MTLRWRCCALGALLGLLLRLDSWSGASGFELTLLHTNDVHARIEETSED
SSKCHEAGSCFAGVARMFTKDGDPEKGDARAVSGRWRSISRHGVVQLLQ
RQRSCAFHEQTWL

In (Ml6) above, the “one or several” amino acids in the amino acid sequence may be any number of amino acids as long as, for example, the protein encoded by the polynucleotide of (Ml6) above has no inosinic acid degrading activity. The “one or several” amino acids in (Ml6) above refer to, for example, 1 to 22 amino acids, 1 to 16 amino acids, 1 to 11 amino acids, 1 to 5 amino acids, 1 to 4 amino acids, 1 to 3 amino acids, 1 or 2 amino acids, or 1 amino acid, in the amino acid sequence of SEQ ID NO: 16.

In (Ml7) above, the “identity” of the amino acid sequence may be any degree of identity as long as, for example, the protein encoded by the polynucleotide of (Ml7) above has no inosinic acid degrading activity. In (Ml7) above, the identity to the amino acid sequence of SEQ ID NO: 16 is, for example, 80% or more, 85% or more, 90% or more, 95% or more, 96% or more, 97% or more, 98% or more, or 99% or more.

In the present disclosure, when the loss of the function of the nt5e gene is caused by suppressing the expression of the nt5e gene, the loss of the function of the nt5e gene can be caused by, for example, introducing a polynucleotide for suppressing the expression of the nt5e gene into a target fish. The method for introducing the polynucleotide is not particularly limited, and can be conducted using, for example, RNA interference, antisense RNA, or a genome editing technology. An expression cassette such as an expression vector that includes the polynucleotide can be introduced into a target fish through, for example, microinjection, a polyethylene glycol method, electroporation, a particle gun method, or the like. The target fish may be any of an egg, a larval fish, a juvenile fish, an immature fish, and an adult fish.

It is possible to suppress a decrease in freshness of the fish of the present disclosure, for example, as compared to that of a fish having the wild-type (normal) nt5e gene. The freshness can be evaluated using a K-value. The K value can be calculated using a formula (1) below based on the measured molar quantities (mol) of adenosine triphosphate (ATP), adenosine diphosphate (ADP), adenosine monophosphate (AMP), inosinic acid (IMP), inosine (HxR), and hypoxanthine (Hx) per weight (mg) of the skeletal muscle of a target fish. The molar quantities of ATP, ADP, AMP, IMP, HxR, and Hx in the skeletal muscle can be quantified using HPLC in accordance with the method described in Example 3, which will be described later.

### (Calculation Formula for K Value)

$$K = ( HxR + Hx ) / ( ATP + A D P + A M P + I M P + HxR + Hx ) \qquad ( 1 )$$

K: index value of freshness
ATP: molar quantity of adenosine triphosphate per weight of muscle
ADP: molar quantity of adenosine diphosphate per weight of muscle
AMP: molar quantity of adenosine monophosphate per weight of muscle
IMP: molar quantity of inosinic acid per weight of muscle
HxR: molar quantity of inosine per weight of muscle
Hx: molar quantity of hypoxanthine per weight of muscle

The fish of the present disclosure has activity of maintaining freshness or activity of suppressing a decrease in freshness to an extent that, for example, when the fish is stored at 4° C. for 2 days after death, the K value decreases by 5% or more, 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, or 99% or more relative to the K value of a fish having the wild-type (normal) nt5e gene stored at 4° C. for 2 days after death. Also, the fish of the present disclosure has activity of maintaining freshness or activity of suppressing a decrease in freshness to an extent that, for example, when the fish is stored at 4° C. for 2 days after death, the K value decreases by 5 to 100%, 5 to 95%, 5 to 90%, 10 to 85%, 10 to 80%, 10 to 75%, 15 to 70%, 15 to 65%, 15 to 60%, 20 to 55%, 20 to 50%, 20 to 45%, 25 to 40%, 25 to 35%, or 25 to 30% relative to the K value of a fish having the wild-type (normal) nt5e gene stored at 4° C. for 2 days after death. Accordingly, the fish of the present disclosure can also be referred to as, for example, “a fish having activity of maintaining freshness” or “a fish having activity of suppressing a decrease in freshness”.

As described above, the fish of the present disclosure is produced by causing a loss-of-function mutation in the wild-type nt5e gene. Accordingly, the fish of the present disclosure can also be referred to as, for example, a “mutant fish”. The fish of the present disclosure may be a fish that is a progeny of the “mutant fish” and has a loss-of-function mutation in the nt5e gene. The fish of the present disclosure can also be referred to as, for example, a mutant fish having, in the base sequence of the nt5e gene, genetic mutation introduced using the above-described method of introducing mutation. The fish of the present disclosure does not encompass, for example, fish obtained only by way of an essentially biological process.

The descriptions of a first production method, a second production method, a screening method, and a third production method, which will be described later, can be applied to the fish production method of the present disclosure.

### <Portion of Fish>

In another aspect, the present disclosure provides a portion of a fish in which accumulation of inosinic acid has been enhanced or accelerated during ripening. The portion of a fish according to the present disclosure is a portion of the fish of the present disclosure.

The fish of the present disclosure may be the above-mentioned first fish of the present disclosure or a second fish of the present disclosure, which will be described later.

An example of the portion of a fish is an edible portion of a fish. Specific examples of the portion of a fish include muscle, a reproductive organ (e.g., the testis or the ovary), skin, the liver, and bone.

### <First Production Method>

In another aspect, the present disclosure provides a fish production method of the present disclosure in which the fish of the present disclosure is used, and a fish reproduction method of the present disclosure. The fish production method of the present disclosure includes a step (a) below:
(a) a step of crossing the fish of the present disclosure with another fish.

With the first production method of the present disclosure, it is possible to produce, for example, an accelerated ripened fish since the fish of the present disclosure is used in the step (a) above.

It is sufficient that, in the step (a) above, the fish of the present disclosure is used as a first parent. As described above, the fish of the present disclosure can also be obtained using, for example, an imparting method, second production method, screening method, and third production method according to the present disclosure, which will be described later. Accordingly, in the first production method of the present disclosure, at least one of the imparting method, second production method, screening method, and third production method according to the present disclosure may be conducted, for example, prior to the step (a) above. In this case, the descriptions of the methods, which will be described later, can be applied to the descriptions of these methods.

In a specific example, the first production method of the present disclosure may include a step (x) or step (y) below.
(x) a step (selection step) of selecting the fish of the present disclosure from test fish
(y) a step (breeding step) of breeding the fish of the present disclosure from a target fish

In the step (x) above, the selection of a fish can also be referred to as “selection of a fish in which the function of the nt5e gene is lost”. Accordingly, for example, the step (x) above can be conducted through a step (x1) and a step (x2) below.
(x1) a detection step of detecting if the function of the nt5e gene is lost in a test fish
(x2) a selection step of selecting the test fish as the fish of the present disclosure when the function of the nt5e gene is lost

When the step (x) above includes the step (x1) and the step (x2) above, the step (x) may be conducted using, for example, the base sequence of the nt5e gene as an indicator or the expression level of the nt5e gene or NT5E protein as an indicator.

When the base sequence of the nt5e gene is used as an indicator, the loss of the function of the nt5e gene may be detected in the step (x1) above by, for example, reading the base sequence of the nt5e gene of a test fish and comparing the base sequence to the base sequence of the corresponding wild-type or loss-of-function nt5e gene. The base sequence can be read using, for example, a sequencer. Then, in the step (x2) above, when, for example, the base sequence of the nt5e gene of the test fish is the base sequence of the wild-type nt5e gene of the corresponding fish into which loss-of-function mutation is introduced, or is the same as the base sequence of the loss-of-function nt5e gene of the corresponding fish, the fish is selected as the fish of the present disclosure. The selection conditions will be described later. Regarding the base sequence of the wild-type nt5e gene, the above-described base sequences of the wild-type nt5e genes of the fish can be referred to. Regarding the base sequence of the loss-of-function nt5e gene, the above-described base sequence of the loss-of-function nt5e genes of the fish can be referred to. The comparison of the base sequences can be conducted using, for example, a base sequence analysis software (e.g., BLAST described above). In the step (x2) above, the region whose base sequence is subjected to the comparison may be the intron region of the nt5e gene or the exon region of the nt5e gene, but the exon region is preferable. When the loss of the function of the nt5e gene is caused by introducing a mutation such as an insertion, a deletion, and/or a substitution of one or more bases into the base sequence of the corresponding wild-type nt5e gene, the step (x1) above may be conducted using, for example, a primer set, probes, or a combination thereof capable of detecting at least one type of mutation. The primer set and probes can be designed using, for example, common techniques in the art based on the type of mutation.

In the step (x2) above, when, for example, an insertion, a deletion, and/or a substitution of one or more bases is introduced into the wild-type nt5e gene, the gene may be determined as a loss-of-function gene. Also, in the step (x2) above, when, for example, a frameshift mutation is introduced into the wild-type nt5e gene, the gene may be determined as a loss-of-function gene. Furthermore, in the step (x2) above, when, for example, the wild-type nt5e gene is partially or completely deleted, the gene may be determined as a loss-of-function gene.

In the step (x2) above, when, for example, the test fish is heterozygous or homozygous for the loss-of-function nt5e gene, the test fish may be selected as the fish of the present disclosure.

When the expression level of the nt5e gene is used as an indicator, the loss of the function of the nt5e gene may be detected in the step (x1) above by, for example, detecting the function of the mRNA of, or the protein encoded by, the nt5e gene of the test fish. Furthermore, in the step (x1) above, the loss of the function of the nt5e gene may be detected by, for example, detecting whether or not the nt5e gene or the protein encoded by the nt5e gene is expressed in the test fish, or detecting the expression level of the nt5e gene or the protein encoded by the nt5e gene.

When the determination in the step (x) above is conducted based on the expression of the protein encoded by the nt5e gene, for example, the expression level of at least either the nt5e gene or the protein encoded by the nt5e gene in the biological sample from the test fish is measured in the step (x1) above. Then, in the step (x2) above, a fish (loss-of-function fish) in which the function of the nt5e gene is lost is selected based on the reference value and the expression level of at least either the nt5e gene or the protein encoded by the nt5e gene in the biological sample from the test fish. Specifically, in the step (x2) above, the loss-of-function fish can be selected from the test fish by, for example, comparing the reference value and the expression level of at least either the nt5e gene or the protein encoded by the nt5e gene in the biological sample from the test fish.

The biological sample from the test fish is not particularly limited, and may be, for example, the individual test fish or a portion thereof, and the skeletal muscle of the fish is preferable. For example, one type of biological sample or two or more types of biological samples may be used in the step (x1) above.

In the step (x1) above, the expression level of the nt5e gene can be measured through, for example, semi-quantitative PCR, quantitative PCR, northern blotting, digital PCR, RNA sequencing (RNAseq) or the like. Also, in the step (x1) above, the expression level of the protein encoded by the nt5e gene can be measured using, for example, a protein quantification method such as a method in which a spectrophotometer is used (e.g., UV absorption method or bicinchoninic acid method), ELISA, western blotting, or the like.

Examples of the reference value above include the expression level of the nt5e gene or the protein encoded by the nt5e gene in the wild-type fish, and the expression level of the nt5e gene or the protein encoded by the nt5e gene in the fish having the loss-of-function nt5e gene. When the expression level of the nt5e gene in the loss-of-function fish is used as the reference value, the loss-of-function fish may be, for example, a fish in which the function of one of the two nt5e genes located on a pair of chromosomes is lost, namely a heterozygous fish, or a fish in which the function of both of the two nt5e genes located on a pair of chromosomes is lost, namely a homozygous fish. The expression level of the nt5e gene or the protein encoded by the nt5e gene, used as the reference value, can be obtained by, for example, measuring in the same manner as that of the biological sample from the test fish, the expression level of the nt5e gene or the protein encoded by the nt5e gene in a biological sample collected under the same conditions as those for the biological sample from the test fish. The reference value may be measured, for example, in advance, or simultaneously with the biological sample from the test fish.

In this case, the method for evaluating if the function of the nt5e gene of the test fish is lost is not particularly limited in the step (x2) above, and can be determined as appropriate in accordance with the type of reference value.

Specifically, when the nt5e gene expression level in the biological sample from the test fish is the same as (not significantly different from) that in a fish that is homozygous for the wild-type nt5e gene and/or is (significantly) higher than that in a fish that is homozygous for the wild-type nt5e gene and/or is (significantly) higher than that in the biological sample from a fish that is homozygous or heterozygous for the loss-of-function nt5e gene, it can be determined that, for example, the function of the nt5e gene is not lost in the test fish. Meanwhile, when the nt5e gene expression level in the biological sample from the test fish is (significantly) lower than that in the biological sample from a fish that is homozygous for the wild-type nt5e gene and/or is the same as (not significantly different from) that in the biological sample from a fish that is homozygous or heterozygous for the loss-of-function nt5e gene and/or is (significantly) lower than that in the biological sample from a fish that is homozygous or heterozygous for the loss-of-function nt5e gene, it can be determined that, for example, the function of the nt5e gene is lost in the test fish.

When the NT5E protein expression level in the biological sample from the test fish is the same as (not significantly different from) that in a fish that is homozygous for the wild-type NT5E protein and/or is (significantly) higher than that in a fish that is homozygous for the wild-type NT5E protein and/or is (significantly) higher than that in the biological sample from a fish that is homozygous or heterozygous for the loss-of-function NT5E protein, it can be determined that, for example, the function of the nt5e gene is not lost in the test fish. Meanwhile, when the NT5E protein expression level in the biological sample from the test fish is (significantly) lower than that in the biological sample from a fish that is homozygous for the wild-type NT5E protein and/or is the same as (not significantly different from) that in the biological sample from a fish that is homozygous or heterozygous for the loss-of-function NT5E protein and/or is (significantly) lower than that in the biological sample from a fish that is homozygous or heterozygous for the loss-of-function NT5E protein, it can be determined that, for example, the function of the nt5e gene is lost in the test fish.

In the step (x2) above, for example, the fish determined as a fish in which the function of the nt5e gene is lost is selected as the fish of the present disclosure.

In the step (x2) above, for example, the genotype of the nt5e gene may be evaluated based on the expression level of the nt5e gene, and specifically, it may be evaluated whether the fish is homozygous for the normal gene, is heterozygous for the nt5e gene and has the normal gene and the loss-of-function gene, or is homozygous for the loss-of-function gene. In this case, the evaluation can be conducted using, as the reference values, a value from a fish (wild-type fish) that is homozygous for the wild-type nt5e gene and a value from a fish (heterozygous fish or homozygous fish) that is heterozygous or homozygous for the loss-of-function nt5e gene. Specifically, when the expression level of the target gene in the test fish is equivalent to that of the target gene in the wild-type fish, the heterozygous fish, or the homozygous fish in the step (x2) above, it can be determined that, for example, the test fish has the same genotype as that of the fish with the equivalent expression level.

The step (y) above can also be referred to as, for example, “a step (functional loss-causing step) of causing the loss of the function of the nt5e gene of the target fish”. The description of a functional loss causing step of the imparting method of the present disclosure, which will be described later, can be applied to the functional loss-causing step.

Next, the fish used as the other parent in the step (a) above is not particularly limited, and a fish with any trait can be used. The fish used as the other parent may be the fish of the present disclosure.

In the step (a) above, the method of crossing the fish of the present disclosure with the other fish is not particularly limited, and a known method can be employed. In the step (a) above, a fish of a progeny line can be obtained by crossing the fish of the present disclosure with the other fish.

The first production method of the present disclosure may further include a step (b) below.
(b) a selection step of selecting a fish in which the function of the ecto-5′-nucleotidase (nt5e) gene is lost from fish obtained in the step (a) above or a progeny line thereof

In the step (b) above, the target fish from which a fish having the loss-of-function nt5e gene is selected may be, for example, the fish obtained in the step (a) above or a progeny line obtained from the fish. Specifically, the target fish may be, for example, F1 fish obtained through the crossing in the step (a) above or a progeny line thereof. The progeny line may be, for example, an inbred progeny or backcross progeny of the F1 fish obtained through the crossing in the step (a) above, or fish obtained by crossing a fish of the progeny line such as the F1 fish with another fish.

In the step (b) above, a fish in which the function of the nt5e gene is lost can be selected by, for example, directly or indirectly confirming the loss of the function of the nt5e gene.

In the step (b) above, the direct confirmation can be determined based on, for example, the inosinic acid level in a biological sample from the obtained F1 fish or a progeny line thereof. Specifically, the loss of the function of the nt5e gene can be evaluated based on the content of inosinic acid in fish meat one day (24 hours) after the death of the fish having the wild-type nt5e gene or loss-of-function nt5e gene. More specifically, the inosinic acid level in the biological sample is measured 24 hours after the death of the test fish in accordance with Example 1, which will be described later. When the inosinic acid level in the biological sample from the test fish is the same as (not significantly different from) that in the biological sample from a fish that is homozygous for the wild-type nt5e gene and/or is (significantly) lower than that in the biological sample from a fish that is homozygous for the wild-type nt5e gene and/or is (significantly) lower than that in the biological sample from a fish that is homozygous or heterozygous for the loss-of-function nt5e gene, it can be determined that, for example, the function of the nt5e gene is not lost in the test fish. Meanwhile, when the inosinic acid level in the biological sample from the test fish is (significantly) higher than that in the biological sample from a fish that is homozygous for the wild-type nt5e gene and/or is the same as (not significantly different from) that in the biological sample from a fish that is homozygous or heterozygous for the loss-of-function nt5e gene and/or is (significantly) higher than that in the biological sample from a fish that is homozygous or heterozygous for the loss-of-function nt5e gene, it can be determined that, for example, the function of the nt5e gene is lost in the test fish.

In addition, in the step (b) above, selection through the indirect confirmation can be conducted through, for example, a step (b1) and a step (b2) below.
(b1) a detection step of detecting if the function of the nt5e gene of the test fish from the step (a) above is lost
(b2) a selection step of selecting the test fish as a fish in which the function of the nt5e gene is lost when the function of the nt5e gene is lost

The selection of a fish in which the function of the nt5e gene is lost in the step (b) above is similar to, for example, the method described in the step (x) above, and the steps (b1) and (b2) above can be conducted similarly to the step (x2) above, respectively.

In the first production method of the present disclosure, it is preferable to further raise the fish selected in the step (b) above. The fish raising conditions and the fish raising method can be determined as appropriate in accordance with, for example, the growth stage of the fish and the breed of the fish. When being raised, for example, the fish may be grown to any growth stage.

As described above, a fish in which the function of the nt5e gene is lost or a progeny line thereof can be selected in the step (b).

The first production method of the present disclosure may further include a collection step of collecting gametes (e.g., eggs and sperms) from the progeny line obtained through the crossing.

### <Second Production Method>

In another aspect, the present disclosure provides a method for producing an accelerated ripened fish. The production method of the present disclosure is a method for producing an accelerated ripened fish, the method including a functional loss-causing step of causing loss of the function of the ecto-5′-nucleotidase (nt5e) gene of a target fish. With the second production method of the present disclosure, it is possible to obtain an accelerated ripened fish. Also, with the second production method of the present disclosure, it is possible to, for example, accelerate the accumulation of inosinic acid during ripening. Accordingly, the second production method of the present disclosure can also be referred to as, for example, “a method for producing a fish in which the accumulation of inosinic acid is accelerated during ripening”.

For example, degradation of inosinic acid into inosine and hypoxanthine during the ripening is suppressed in the fish of the present disclosure, and thus the freshness is maintained after storage for the same period of time. Accordingly, the enhancing method of the present disclosure can also be referred to as, for example, “a method for producing a fish whose freshness is maintained”, “a method for producing a fish in which a decrease in freshness is suppressed”, “a method for maintaining freshness of a fish”, or “a method for suppressing a decrease in freshness of a fish”.

In the present disclosure, as described above, the loss of the function of the nt5e gene may be caused by introducing loss-of-function mutation into the nt5e gene, or introducing a polynucleotide for suppressing the expression of the nt5e gene, or crossing a target fish with the fish of the present disclosure (i.e., by introducing the loss-of-function nt5e gene through crossing). When the loss of the function is caused through the crossing, the imparting method of the present disclosure can be conducted, for example, similarly to the first production method of the present disclosure.

When the loss-of-function mutation is introduced into the nt5e gene, the loss-of-function mutation is introduced into, for example, the nt5e gene of a target fish in the functional loss-causing step. The target fish has the nt5e gene, for example, on each of a pair of chromosomes. Accordingly, in the functional loss-causing step, for example, the loss of the function of the nt5e gene may be caused on one of the pair of chromosomes of the target fish, or the loss of the function of the nt5e gene may be caused on both of the chromosomes, but the latter is preferable.

The loss of the function of the nt5e gene can be caused by, for example, introducing mutation as described above. The description above can be applied to the mutation, and a nonsense mutation or a frameshift mutation is preferable. The loss-of-function mutation may be introduced by, for example, introducing a deletion, a substitution, an insertion, and/or an addition of one or several bases into the gene (the base sequence of the gene), and it is preferable that the loss-of-function mutation is introduced through partial or complete deletion of the wild-type nt5e gene.

In the functional loss-causing step, the region of the nt5e gene into which a loss-of-function mutation is introduced may be the intron region of the nt5e gene or the exon region of the nt5e gene, but the exon region is preferable.

The loss of the function of the nt5e gene can be caused by, for example, introducing a mutation into the nt5e gene of a target fish using a routine procedure. A method for introducing the mutation can be a method such as homologous recombination or a genome-editing technology in which ZFN, TALEN, CRISPR-CAS9, CRISPR-CPF1, or the like is used. The method for introducing the mutation with the genome editing technology can be conducted with reference to, for example, Example 1 below. The method for introducing the mutation may be, for example, a random mutagenesis. Examples of the random mutagenesis include: irradiation with α-rays, β-rays, γ-rays, X-rays, or the like; chemical treatment with a mutagenic agent such as ethyl methanesulfonate (EMS) or ethynylnitrosourea (ENU); and heavy ion beam treatment. Note that the above-described methods for introducing mutation may be conducted using, for example, commercially available kits and the like.

The target fish may be any of an egg, a larval fish, a juvenile fish, an immature fish, and an adult fish.

It is preferable that, in the second production method of the present disclosure, fish in which the loss-of-function mutation is introduced into the nt5e gene are selected after the functional loss-causing step. The selection target fish may be, for example, fish obtained in the functional loss-causing step or fish of a progeny line thereof. The selection can be conducted, for example, similarly to the step (x) above, and the description of the step (x) can be applied thereto.

Next, when the polynucleotide for suppressing the expression of the nt5e gene is introduced, the method for introducing the polynucleotide is not particularly limited, and can be conducted using, for example, RNA interference, antisense RNA, or a genome editing technology. An expression cassette such as an expression vector that includes the polynucleotide can be introduced into a target fish through, for example, microinjection, a polyethylene glycol method, electroporation, a particle gun method, or the like. The target fish may be any of an egg, a larval fish, a juvenile fish, an immature fish, and an adult fish.

### <Method for Enhancing Inosinic Acid Content>

In another aspect, the present disclosure provides a method capable of enhancing an inosinic acid content during ripening of a fish or fish meat. The enhancing method of the present disclosure is a method for enhancing an inosinic acid content during ripening of fish meat, including a ripening step of ripening a fish or fish meat, wherein the fish meat is the fish of the present disclosure, fish meat of the fish of the present disclosure, and/or fish meat in an edible portion of the fish of the present disclosure. With the enhancing method of the present disclosure, it is possible to enhance the inosinic acid content in the fish or fish meat during ripening. In the enhancing method of the present disclosure, for example, an increase in the amount of inosinic acid during ripening is enhanced as compared to that of a fish that is homozygous for the wild-type nt5e gene, and thus the same inosinic acid content can be reached faster. Accordingly, the enhancing method of the present disclosure can also be referred to as, for example, “a method for accelerating ripening of a fish or fish meat” or “a method for accelerating accumulation of inosinic acid in a fish or fish meat”.

In the ripening step above, for example, general ripening conditions for the fish or fish meat can be applied to the method for ripening the fish or fish meat. Specifically, the temperature during the ripening is, for example, 1 to 10° C. The period of the ripening (ripening period) is, for example, 0.1 to 31 days, 0.5 to 20 days, or 1 to 3 days.

### <Screening Method>

In another aspect, the present disclosure provides a method for screening an accelerated ripened fish. The method for screening an accelerated ripened fish according to the present disclosure includes a selection step of selecting, from a test sample of fish, a test fish in which the function of the ecto-5′-nucleotidase (nt5e) gene is lost as an accelerated ripened fish. With the present disclosure, it is possible to screen an accelerated ripened fish. The screening method of the present invention can also be referred to as, for example, “a method for screening a fish having an ability to enhance (accelerate) accumulation of inosinic acid”.

For example, degradation of inosinic acid into inosine and hypoxanthine during the ripening is suppressed in the fish of the present disclosure, and thus the freshness is maintained after storage for the same period of time. Accordingly, the screening method of the present disclosure can also be referred to as, for example, “a method for screening a fish whose freshness is maintained” or “a method for screening a fish in which a decrease in freshness is suppressed”.

In the screening method of the present disclosure, the selection step can be conducted similarly to the step (x) above, and the description of the step (x) can be applied thereto.

### <Third Production Method>

In another aspect, the present disclosure provides a method for producing an accelerated ripened fish. The method for producing a fish according to the present disclosure includes a screening step of screening, from test fish, a test fish in which the function of the nt5e gene is lost, and the screening step is conducted in accordance with the method for screening an accelerated ripened fish according to the present disclosure. With the third production method of the present disclosure, it is possible to produce an accelerated ripened fish because a fish having the loss-of-function nt5e gene can be screened.

### <Second Fish>

In another aspect, the present disclosure provides an accelerated ripened fish. A fish (hereinafter also referred to as a “second fish”) of the present disclosure is obtained using the first production method, second production method, or third production method of the present disclosure. With the fish of the present disclosure, it is possible to provide an accelerated ripened fish. Also, with the present disclosure, the accumulation of inosinic acid in fish meat can be accelerated after the death of a fish, and therefore, it is expected that a fish that maintains a firm texture and has umami flavor is obtained.

### <Detection Method>

In another aspect, the present disclosure provides a method capable of detecting an ability of a fish to be ripened with acceleration. The detection method of the present disclosure includes a detection step of detecting if the function of the ecto-5′-nucleotidase (nt5e) gene is lost in a test fish. With the detection method of the present disclosure, it is possible to detect whether or not a test fish will be ripened with acceleration during ripening or whether or not accumulation of inosinic acid will be enhanced during ripening. Accordingly, the detection method of the present disclosure can also be referred to as, for example, “a method for screening a fish having an ability to be ripened with acceleration” or “a method for screening a fish having an ability to enhance (accelerate) accumulation of inosinic acid”.

The detection method of the present disclosure includes, for example, a detection step of detecting functional deficiency-causing mutation in the nt5e gene of a test fish. For example, the description of the step (x) or indirect selection in the selection step of the first production method of the present disclosure can be applied to the detection step. Specifically, in the detection step, for example, the gene expression of the nt5e gene or the base sequence thereof is detected.

It is preferable that the detection method of the present disclosure further includes a determination step of determining whether the nt5e gene of the test fish is the wild-type nt5e gene or loss-of-function nt5e gene based on the above-mentioned gene expression or base sequence. In the determination step, the determination can be performed by, for example, comparing the nt5e gene of the test fish with the wild-type nt5e gene of the corresponding fish. Specifically, in the determination step above, when the nt5e gene of the test fish has the same base sequence as that of the wild-type nt5e gene of the corresponding fish, or has mutation other than the loss-of-function mutation, or has a base sequence different from that of the loss-of-function nt5e gene of the corresponding fish, the nt5e gene of the test fish can be determined as the wild-type nt5e gene. Meanwhile, in the determination step above, when the nt5e gene of the test fish corresponds to the wild-type nt5e gene of the corresponding fish and has the loss-of-function mutation, or has the same base sequence as that of the loss-of-function nt5e gene of the corresponding fish, the nt5e gene of the test fish can be determined as the loss-of-function nt5e gene.

### <Processed Food>

In another aspect, the present disclosure provides processed food containing the fish of the present disclosure. The fish processed food of the present disclosure contains the fish of the present disclosure.

A fish to be processed for the fish processed food of the present disclosure may be the first fish of the present disclosure or the second fish of the present disclosure.

The “processing” for the processed food of the present disclosure is not particularly limited and means, for example, any treatment for a fish. Specific examples of the processing include cutting, slicing, mincing, straining, drying, canning, bottling, washing, packaging, freezing, heating, and flavoring. One type or a plurality of types of processing may be performed for production of the processed food. Also, the same processing may be performed once or a plurality of times in the production of the processed food.

### EXAMPLES

Hereinafter, the present disclosure will be described in detail by way of examples, but the present disclosure is not limited to aspects described in the examples.

### Example 1

Medakas in which the function of the nt5e gene was lost were bred and used to check that accumulation of inosinic acid was enhanced as compared to that of wild-type medakas, that is, the nt5e gene encoded an inosinic acid-degrading enzyme.

### (1) Production of Fertilized Eggs

Sexually mature medakas were placed in the same aquarium, and female medakas and male medakas were separated from each other using a partition board or the like in the evening of the day before microinjection. On the day of microinjection, the partition board was removed to allow natural mating of the medakas, and thus single-cell fertilized eggs were obtained. The collected eggs were used for microinjection after filaments attached to the egg membranes were removed with tweezers.

### (2) Breeding of Loss-of-Function Medakas

A mutation was introduced using CRISPR-Cas9 with reference to Reference 1 below. A protein reagent purchased from Integrated DNA Technologies, Inc. was used as Cas9.
Reference 1: Satoshi Ansai et. al, “Targeted mutagenesis using CRISPR/Cas system in medaka”, Biology Open, 2014, vol. 3, pages 362-371

Single-guide RNAs (sgRNAs) were synthesized using a cloning-free method requiring no vectors. Template DNAs for synthesis were prepared by PCR using three oligonucleotides (OligoA-gRNA1 or OligoA-sgRNA2/OligoB/OligoC). sgRNA1 and sgRNA2 were synthesized using the template DNAs and a kit (CUGA® 7 gRNA Synthesis Kit, manufactured by Nippon Gene Co., Ltd.) and were then purified using an RNA purification kit (RNeasy Plus Mini Kit, manufactured by Qiagen). The target sites on the genome targeted by the sgRNAs are target sequences below that are present in exon 1 or exon 9 of the ect-5′-nucleotidase (nt5e) gene. There are 10 types of nt5 genes in the medaka genome database. Out of these types, nt5e genes classified as the extracellular type are reported to generally have a high affinity for inosinic acid (see Reference 2). Medakas have two types of nt5e genes, and the nt5e gene (ENSORLG00000014932) on chromosome 22, which is revealed from the results of synteny analysis to be conserved among a wide variety of fish, was targeted. In the target sequences below, the underlined three bases at the 3′-terminus correspond to the protospacer adjacent motif (PAM) sequence.
Reference 2: H Zimmermann, “5′-Nucleotidase: molecular structure and functional aspects”, Biochem J. 1992 Jul. 15; 285 (Pt 2)(Pt 2):345-65.

(OligoA-sgRNA1)
(SEQ ID NO: 17)
CTAATACGACTCACTATAGCAGGATGTTCACCAAAGTGAGTTTTAGAGC
TAGAAATAGCA-3′
(OligoA-sgRNA2)
(SEQ ID NO: 18)
5′-CTAATACGACTCACTATAGGTGATGCGGCCTTCCACTGCGTTTTAG
AGCTAGAAATAGCA-3′
(OligoB)
(SEQ ID NO: 19)
5′-AAAAGCACCGACTCGGTGCCACTTTTTCAAGTTGATAACGGACTAG
CCTTATTTTAACTTGCTATTTCTAGCTCTAAAAC-3′
(OligoC)
(SEQ ID NO: 20)
5′-AAAAGCACCGACTCGGTGCC-3′
(Target Sequence on Exon 1: sgRNA1)
(SEQ ID NO: 21)
5′-CAGGATGTTCACCAAAGTGACGG-3′
(Target Sequence on Exon 9: sgRNA2)
(SEQ ID NO: 22)
5′-GTGATGCGGCCTTCCACTGCAGG-3′

A mutation was introduced into the nt5e gene by introducing 500 ng/μL Cas9 protein and 100 ng/μL sgRNA1 into the cytoplasm of the single-cell fertilized egg obtained in (1) above using the microinjection method (Case 1). Also, mutation was introduced into the nt5e gene by introducing 500 ng/μL Cas9 protein, 100 ng/μL sgRNA1, and 100 ng/μL sgRNA2 into the cytoplasm of the single-cell fertilized egg obtained in (1) above using the microinjection method (Case 2). Then, second-generation medakas were obtained by mating the medaka into which the mutation had been introduced with a wild-type medaka. DNAs extracted from the caudal fins of the second-generation medakas were analyzed, and individuals in which base deletion was observed in the nt5e gene were selected through PCR and base sequence analysis. Individuals into which functional deficiency causing mutation had been introduced into the nt5e gene were obtained by mating the above-mentioned individuals. In Case 1, the individuals, into which the functional deficiency causing mutation had been introduced into the nt5e gene, belonged to a 2-base deletion line (Δ2). In Case 2, the individuals, into which the functional deficiency causing mutation had been introduced into the nt5e gene, belonged to a 5521-base deletion line (Δ5521).

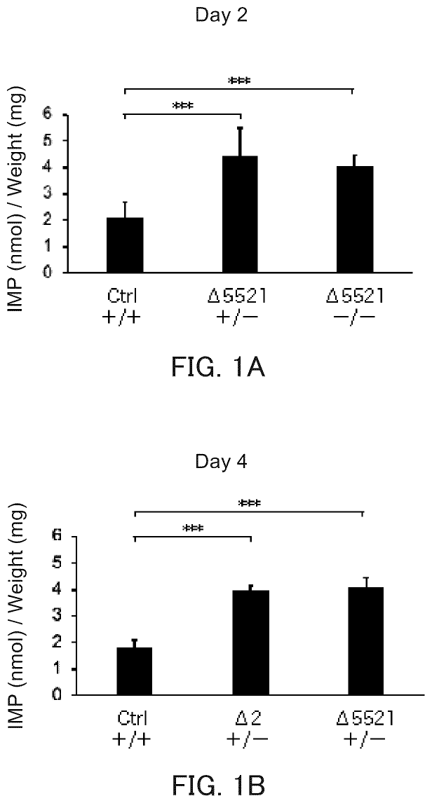

After the production above, the fertilized eggs were cultured and hatched, and rearing was conducted in accordance with an ordinary aquaculture method. The individuals were killed instantly 3 months after the hatching and were stored at 4° C. for 2 or 4 days for ripening. After the storage, the skeletal muscle was collected from each individual and the weight of the skeletal muscle was measured. After the measurement, 10% perchloric acid was added and the skeletal muscle was homogenized. After the homogenization, the homogenate was centrifuged at 12900 rpm (or 15000×g) for 10 minutes. After the centrifugation, the supernatant fraction was collected. After the collection, 1 N KOH was added to the supernatant fraction to neutralize it, and the mixture was centrifuged at 12900 rpm (or 15000×g) for 10 minutes. After the centrifugation, the supernatant fraction was collected. Thereafter, the supernatant fraction was diluted to a predetermined volume with distilled water, and the inosinic acid level (nmol/mg skeletal muscle) per weight of the skeletal muscle was measured using HPLC. In this measurement, a C18 reversed phase column (4.6 mm I.D.×250 mm, OTD-80 Ts, manufactured by Tosoh Corporation) was used. The results are shown in Tables 2 and 3 below and FIG. 1.

TABLE 2

| | Control (+/+) | Δ2 (+/−) | Δ2 (−/−) | Δ5521 (+/−) | Δ5521 (−/−) |
|---|---|---|---|---|---|
| Mean | 2.10 | 3.95 | 3.67 | 4.43 | 4.02 |
| SD | 0.58 | 0.27 | 0.37 | 1.07 | 0.45 |
| SE | 0.22 | 0.10 | 0.14 | 0.40 | 0.17 |

TABLE 3

| | Control (+/+) | Δ2 (+/−) | Δ2 (−/−) | Δ5521 (+/−) |
|---|---|---|---|---|
| Mean | 1.83 | 3.97 | 3.58 | 4.07 |
| SD | 0.28 | 0.19 | 0.25 | 0.36 |
| SE | 0.10 | 0.07 | 0.09 | 0.14 |

FIG. 1 shows graphs illustrating the inosinic acid levels in the medakas in which the function of the nt5e gene is lost. FIG. 1(A) shows the inosinic acid levels in the medakas after the ripening period of 2 days, and FIG. 1(B) shows the inosinic acid levels in the medakas after the ripening period of 4 days. In FIG. 1, the horizontal axes indicate the types of individuals, and the vertical axes indicate the inosinic acid levels. As shown in FIG. 1(A) and Table 2 above, in both the hetero-knockout A2 medakas and the homo-knockout A2 medakas in Case 1 as well as in both the hetero-knockout A5521 medakas and the homo-knockout A5521 medakas in Case 2, the inosinic acid level was significantly higher after the ripening period of 2 days as compared to that of the control medakas (wild-type medakas). Also, as shown in FIG. 1(B) and Table 3 above, in both the hetero-knockout A2 medakas and the homo-knockout A2 medakas in Case 1 as well as in the hetero-knockout A5521 medakas in Case 2, the inosinic acid level was significantly higher after the ripening period of 4 days as compared to that of the control medakas (wild-type medakas). It was found from these results that the inosinic acid level was enhanced after the ripening period of 2 days and the ripening period of 4 days in the medakas of the present disclosure as compared to that of the wild-type medakas, that is, the activity of degrading inosinic acid was suppressed due to the introduction of the loss-of-function mutation into the nt5e gene.

### Example 2

Red seabreams in which the function of the nt5e gene was lost were bred and used to check that the activity of degrading inosinic acid was suppressed as compared to that of wild-type red seabreams.

A mutation was introduced into the nt5e gene in the same manner as described above, except that female and male red seabreams were used instead of female and male medakas and different sgRNAs were used. In the red seabreams, the target site on the genome targeted by the sgRNA is a target sequence below that is present in exon 6 of the Pm-nt5e gene. In the target sequence below, the underlined three bases at the 3′-terminus correspond to the protospacer adjacent motif (PAM) sequence.

(Target Sequence on Exon 6: Pm-Nt5-sgRNA)
(SEQ ID NO: 23)
5′-TCAGGAGGTGGGAAAGACTCTGG-3′

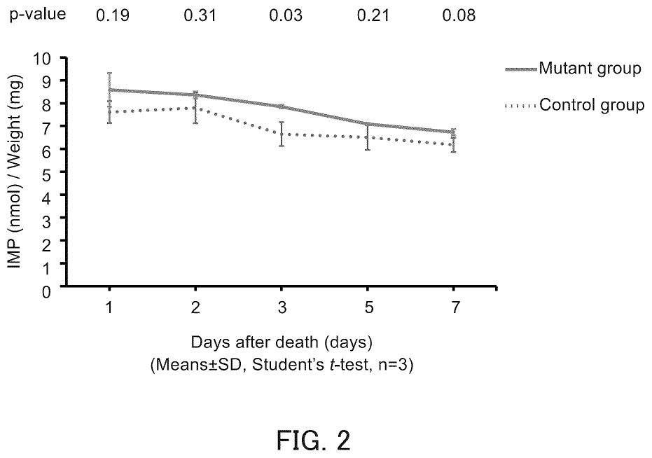

Next, the same procedure as that of Example 1 above was conducted, except that the fertilized red seabream eggs with the mutation introduced were used. The individuals were killed instantly 6 months after the hatching, and the inosinic acid level was measured over time 1, 2, 3, 5, or 7 days after the death. Specifically, the skeletal muscle was collected from each individual and the weight of the skeletal muscle was measured. After the measurement, 10% perchloric acid was added and the skeletal muscle was homogenized. After the homogenization, the homogenate was centrifuged at 12,900 rpm (or 15,000×g) for 10 minutes. After the centrifugation, the supernatant fraction was collected. After the collection, 1 N KOH was added to the supernatant fraction to neutralize it, and the mixture was centrifuged at 12,900 rpm (or 15,000×g) for 10 minutes. After the centrifugation, the supernatant fraction was collected. Thereafter, the supernatant fraction was diluted to a predetermined volume with distilled water, and the inosinic acid level was measured using HPLC. In this measurement, a C18 reversed phase column (4.6 mm I.D.×250 mm, OTD-80 Ts, manufactured by Tosoh Corporation) was used. The results are shown in FIG. 2.

FIG. 2 is a graph illustrating the inosinic acid levels in the red seabreams in which the function of the nt5e gene is lost. In FIG. 2, the horizontal axis indicates the days after death (ripening period), and the vertical axis indicates the inosinic acid level. As shown in FIG. 2, the inosinic acid levels were higher at all the time points in the red seabreams of Example 2 (mutant group) as compared to that of the control red seabreams (wild-type red seabreams; control group). It was found from these results that the activity of degrading inosinic acid was suppressed in the red seabreams of the present disclosure as compared to that of the wild-type red seabreams. Although the results of Example 2 were obtained from chimeric red seabreams, it is found that the activity of degrading inosinic acid was suppressed. Accordingly, it is presumed that, similarly to the medakas of Example 1 above, the accumulation of inosinic acid was enhanced due to the red seabreams being heterozygous or homozygous for the loss-of-function nt5e gene.

### Example 3

Tilapias in which the function of the nt5e gene was lost were bred and used to check that the activity of degrading inosinic acid was suppressed and the freshness was maintained as compared to that of wild-type tilapias.

A mutation was introduced into the nt5e gene in the same manner as described above, except that female and male tilapias were used instead of female and male medakas and different sgRNAs were used. In the tilapias, the target site on the genome targeted by the sgRNA is a target sequence below that is present in exon 1 of the nt5e gene (the base sequence from position 217 to position 239 in the base sequence of SEQ ID NO: 5). In the target sequence below, the underlined three bases at the 5′-terminus correspond to the protospacer adjacent motif (PAM) sequence.

(Target Sequence on Exon 1: sgRNA-t)
(SEQ ID NO: 24)
5′-CCCTGCTTCGCTGGTGTGGCCAG-3′

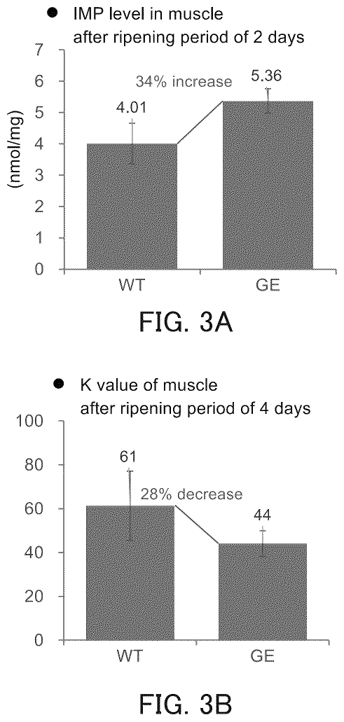

Next, the same procedure as that of Example 1 above was conducted, except that the fertilized tilapia eggs with the mutation introduced were used. The individuals were killed instantly 50 days after the hatching and stored at 4° C. for 2 or 4 days for ripening. After the storage, the levels of nucleic acids (ATP, ADP, AMP, IMP, HxR, and Hx) in each individual were measured. Specifically, the skeletal muscle was collected from each individual and the weight of the skeletal muscle was measured. After the measurement, 10% perchloric acid was added and the skeletal muscle was homogenized. After the homogenization, the homogenate was centrifuged at 12900 rpm (or 15000×g) for 10 minutes. After the centrifugation, the supernatant fraction was collected. After the collection, 1 N KOH was added to the supernatant fraction to neutralize it, and the mixture was centrifuged at 12900 rpm (or 15000×g) for 10 minutes. After the centrifugation, the supernatant fraction was collected. Thereafter, the supernatant fraction was diluted to a predetermined volume with distilled water, and the above-mentioned nucleic acid levels were measured using HPLC. Also, the K value, which is an index of freshness, was calculated using the nucleic acid measurement values and Formula (1) below. The following formula was used for the calculation above. In this measurement, a C18 reversed phase column (4.6 mm I.D.×250 mm, OTD-80 Ts, manufactured by Tosoh Corporation) was used. The results are shown in FIG. 3.

### (Calculation Formula for K Value)

$$K = ( HxR + Hx ) / ( ATP + A D P + A M P + I M P + HxR + Hx ) \qquad ( 1 )$$

K: index value of freshness
ATP: molar quantity of adenosine triphosphate per weight of muscle
ADP: molar quantity of adenosine diphosphate per weight of muscle
AMP: molar quantity of adenosine monophosphate per weight of muscle
IMP: molar quantity of inosinic acid per weight of muscle
HxR: molar quantity of inosine per weight of muscle
Hx: molar quantity of hypoxanthine per weight of muscle

FIG. 3 shows graphs illustrating the inosinic acid levels in and the K values of tilapias in which the function of the nt5e gene is lost. FIG. 3(A) shows the inosinic acid levels in the tilapias after the ripening period of 2 days, and FIG. 3(B) shows the K values of the tilapias after the ripening period of 4 days. In FIG. 3(A), the horizontal axis indicates the types of individuals, and the vertical axis indicates the inosinic acid level. In FIG. 3(B), the horizontal axis indicates the types of individuals, and the vertical axis indicate the K value. As shown in FIG. 3(A), the inosinic acid level was higher by 34% in the tilapias of Example 3 (GE) as compared to that of the control tilapias (wild-type tilapias; WT) after the ripening period of 2 days. Also, as shown in FIG. 3(B), the K value was lower by 28% in the tilapias of Example 3 (GE) as compared to that of the control tilapias (wild-type tilapias; WT) after the ripening period of 4 days. It was found from these results that, in the tilapias of the present disclosure, the activity of degrading inosinic acid was suppressed after the ripening period of 2 days and the freshness was maintained after the ripening period of 4 days, as compared to those of the wild-type tilapias.

As a result, it was found that, in the fish of the present disclosure, the activity of degrading inosinic acid was suppressed and the freshness was maintained, as compared to those of the wild-type fish.

### Example 4

Bastard halibuts in which the function of the nt5e gene was lost were bred and used to check that the activity of degrading inosinic acid was suppressed and the freshness was maintained as compared to those of wild-type bastard halibuts.

Mutation was introduced into the nt5e gene in the same manner as described above, except that female and male bastard halibuts were used instead of female and male medakas and different sgRNAs were used. In the bastard halibuts, the target site on the genome targeted by the sgRNA is a target sequence below that is present in exon 4 of the nt5e gene (the base sequence from position 605 to position 627 in the base sequence of SEQ ID NO: 11). In the target sequence below, the underlined three bases at the 3-terminus correspond to the protospacer adjacent motif (PAM) sequence.

(Target Sequence on Exon 4: sgRNA-p)
(SEQ ID NO: 27)
5′-CTTCAACGGAGGCGGCGTCCGGG-3′

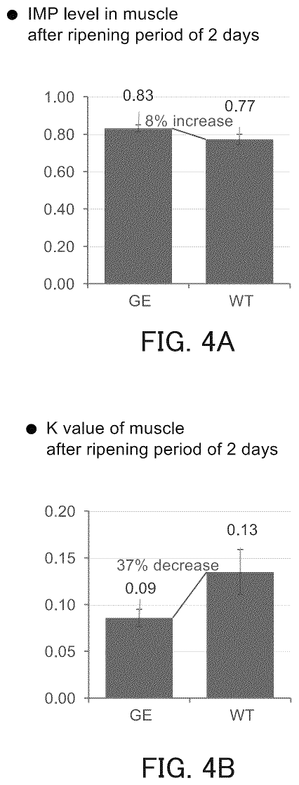

Next, the same procedure as that of Example 1 above was conducted, except that the fertilized bastard halibut eggs with the mutation introduced were used. The individuals were killed instantly 546 days after the fertilization and stored at 4° C. for 2 days for ripening. After the storage, the levels of nucleic acids (ATP, ADP, AMP, IMP, HxR, and Hx) in each individual were measured. Specifically, the measurement was conducted similarly to the method described in Example 2 above. The results are shown in FIG. 4.

FIG. 4 shows graphs illustrating the inosinic acid levels in and the K values of bastard halibuts in which the function of the nt5e gene is lost. FIG. 4(A) shows the inosinic acid levels in the bastard halibuts after the ripening period of 2 days, and FIG. 4(B) shows the K values of the bastard halibuts after the ripening period of 2 days. In FIG. 4(A), the horizontal axis indicates the types of individuals, and the vertical axis indicates the inosinic acid level. In FIG. 4(B), the horizontal axis indicates the types of individuals, and the vertical axis indicate the K value. As shown in FIG. 4(A), the inosinic acid level was higher by 8% in the bastard halibuts of Example 4 (GE) as compared to that of the control bastard halibuts (wild-type bastard halibuts; WT) after the ripening period of 2 days. As shown in FIG. 4(B), the K value was lower by 37% in the bastard halibuts of Example 4 (GE) as compared to that of the control bastard halibuts (wild-type bastard halibuts; WT) after the ripening period of 2 days. It was found from these results that, in the bastard halibuts of the present disclosure, the activity of degrading bastard halibut acid was suppressed and the freshness was maintained after the ripening period of 2 days, as compared to those of the wild-type bastard halibuts.

As a result, it was found that, in the fish of the present disclosure, the activity of degrading inosinic acid was suppressed and the freshness was maintained, as compared to those of the wild-type fish.

As described above, the present disclosure has been described with reference to the embodiments and the examples, but the present disclosure is not limited to the above-described embodiments and examples. Various modifications that can be understood by a person skilled in the art can be made in the configurations and details of the present disclosure without departing from the scope of the present disclosure.

The contents of the patents, patent applications, and references cited herein are incorporated herein by reference in their entireties as if specifically set forth herein.

The present application claims the benefit of priority from Japanese Patent Application No. 2022-096417 filed on Jun. 15, 2022, the entire disclosure of which is incorporated herein.

### <Supplementary Notes>

Some or all of the embodiments and the examples given above can be described as in the following supplementary notes, but the scope of the present disclosure is not limited thereto.

### <Fish>

### (Supplementary Note 1)

A fish in which a function of an ecto-5′-nucleotidase (nt5e) gene is lost.

### (Supplementary Note 2)

The fish according to Supplementary Note 1, comprising a loss-of-function nt5e gene,

wherein the loss-of-function nt5e gene is a mutant gene having a base sequence consisting of a base sequence of a normal nt5e gene with a deletion, a substitution, an insertion, and/or an addition of one or several bases.

### (Supplementary Note 3)

The fish according to Supplementary Note 2,

wherein the loss-of-function nt5e gene is a mutant gene having a base sequence consisting of the base sequence of the normal nt5e gene with deletion of at least some bases.

### (Supplementary Note 4)

The fish according to any one of Supplementary Notes 1 to 3, comprising a loss-of-function nt5e gene,

wherein the loss-of-function nt5e gene is a mutant gene having a base sequence consisting of a base sequence of a normal nt5e gene with frameshift mutation.

### (Supplementary Note 5)

The fish according to any one of Supplementary Notes 1 to 4, comprising a loss-of-function nt5e gene,

wherein the loss-of-function nt5e gene is a mutant gene having a base sequence consisting of a base sequence of a normal nt5e gene with nonsense mutation.

### (Supplementary Note 6)

The fish according to any one of Supplementary Notes 1 to 5, comprising a loss-of-function nt5e gene,

wherein the loss-of-function nt5e gene is a gene having a base sequence consisting of a base sequence of a normal nt5e gene with mutation in a first exon.

### (Supplementary Note 7)

The fish according to any one of Supplementary Notes 2 to 6,

wherein the fish is a red seabream (Pagrus major), and

a normal nt5e gene of the red seabream is a gene that includes a polynucleotide (Pn) below:
(Pn) any of polynucleotides (Pn1) to (Pn7) below:
(Pn1) a polynucleotide having a base sequence of SEQ ID NO: 1;
(Pn2) a polynucleotide having a base sequence of (Pn1) above that encodes a protein having an activity of degrading inosinic acid with an amino acid sequence in which a no acid is deleted, substituted, inserted, and/or added; and
(Pn7) a polynucleotide encoding a protein that has an activity of degrading inosinic acid and that has an amino acid sequence having 80% or more identity to an amino acid sequence of SEQ ID NO: 2.

### (Supplementary Note 8)

The fish according to any one of Supplementary Notes 2 to 7,

wherein the fish is a red seabream (Pagrus major), and

the red seabream includes, as a loss-of-function nt5e gene, a mutant gene having mutation in at least one of exon 1 and exon 6 of a normal nt5e gene.

### (Supplementary Note 9)

The fish according to Supplementary Note 8,

wherein the red seabream includes, as a loss-of-function nt5e gene, a mutant gene that has mutation in the exon 6 of the normal nt5e gene and that has a base sequence consisting of a base sequence of the normal nt5e gene with frameshift mutation or nonsense mutation.

### (Supplementary Note 10)

The fish according to any one of Supplementary Notes 2 to 9,

wherein the fish is a red seabream (Pagrus major), and

the red seabream includes, as a loss-of-function nt5e gene, a mutant gene having mutation in bases from position 1014 to position 1036 of a base sequence of SEQ ID NO: 1.

### (Supplementary Note 11)

The fish according to any one of Supplementary Notes 2 to 6,

wherein the fish is a tiger puffer (Takifugu rubripes), and

a normal nt5e gene of the tiger puffer is a gene that includes a polynucleotide (Pt) below:
(Pt) any of polynucleotides (Pt1) to (Pt7) below:
(Pt1) a polynucleotide having a base sequence of SEQ ID NO: 3;
(Pt2) a polynucleotide that encodes a protein having an activity of degrading inosinic acid and that has a base sequence consisting of the base sequence of (Pt1) above with a deletion, a substitution, an insertion, and/or an addition of one or several bases;
(Pt3) a polynucleotide that encodes a protein having an activity of degrading inosinic acid and that has a base sequence having 80% or more identity to the base sequence of (Pt1) above;
(Pt4) a polynucleotide that encodes a protein having an activity of degrading inosinic acid and that has a base sequence complementary to a polynucleotide capable of hybridizing to the polynucleotide having the base sequence of (Pt1) above under a stringent condition;
(Pt5) a polynucleotide encoding a protein having an amino acid sequence of SEQ ID NO: 4;
(Pt6) a polynucleotide encoding a protein that has an activity of degrading inosinic acid and that has an amino acid sequence consisting of the amino acid sequence of SEQ ID NO: 4 with a deletion, a substitution, an insertion, and/or an addition of one or several amino acids; and
(Pt7) a polynucleotide encoding a protein that has an activity of degrading inosinic acid and that has an amino acid sequence having 80% or more identity to the amino acid sequence of SEQ ID NO: 4.

### (Supplementary Note 12)

The fish according to any one of Supplementary Notes 2 to 6 and 11,

wherein the fish is a tiger puffer (Takifugu rubripes), and

the tiger puffer includes, as a loss-of-function nt5e gene, a mutant gene having mutation in exon 1 of a normal gene of a normal nt5e gene.

### (Supplementary Note 13)

The fish according to Supplementary Note 12,

wherein the tiger puffer includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 14)

The fish according to any one of Supplementary Notes 2 to 6 and 11 to 13,

wherein the fish is a tiger puffer (Takifugu rubripes), and

the tiger puffer has, as a loss-of-function nt5e gene, mutation in bases from position 131 to position 153 of a base sequence of SEQ ID NO: 3.

### (Supplementary Note 15)

The fish according to any one of Supplementary Notes 2 to 6,

wherein the fish is a tilapia (Oreochromis niloticus), and

a normal nt5e gene of the tilapia is a gene that includes a polynucleotide (Po) below:
(Po) any of polynucleotides (Po1) to (Po7) below:
(Po1) a polynucleotide having a base sequence of SEQ ID NO: 5;
(Po2) a polynucleotide that encodes a protein having an activity of degrading inosinic acid and that has a base sequence consisting of the base sequence of (Po1) above with a deletion, a substitution, an insertion, and/or an addition of one or several bases;
(Po3) a polynucleotide that encodes a protein having an activity of degrading inosinic acid and that has a base sequence having 80% or more identity to the base sequence of (Po1) above;
(Po4) a polynucleotide that encodes a protein having an activity of degrading inosinic acid and that has a base sequence complementary to a polynucleotide capable of hybridizing to the polynucleotide having the base sequence of (Po1) above under a stringent condition;
(Po5) a polynucleotide encoding a protein having an amino acid sequence of SEQ ID NO: 6;
(Po6) a polynucleotide encoding a protein that has an activity of degrading inosinic acid and that has an amino acid sequence consisting of the amino acid sequence of SEQ ID NO: 6 with a deletion, a substitution, an insertion, and/or an addition of one or several amino acids; and
(Po7) a polynucleotide encoding a protein that has an activity of degrading inosinic acid and that has an amino acid sequence having 80% or more identity to the amino acid sequence of SEQ ID NO: 6.

### (Supplementary Note 16)

The fish according to any one of Supplementary Notes 2 to 6 and 15,

wherein the fish is a tilapia (Oreochromis niloticus), and

the tilapia includes, as a loss-of-function nt5e gene, a mutant gene having mutation in exon 1 of a normal gene of an nt5e gene.

### (Supplementary Note 17)

The fish according to Supplementary Note 16,

wherein the tilapia includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 18)

The fish according to any one of Supplementary Notes 2 to 6 and 15 to 17,

wherein the fish is a tilapia (Oreochromis niloticus), and

the tilapia has, as a loss-of-function nt5e gene, mutation in bases from position 217 to position 239 of a base sequence of SEQ ID NO: 5.

### (Supplementary Note 19)

The fish according to any one of Supplementary Notes 2 to 6,

wherein the fish is a bastard halibut (Paralichthys olivaceus), and

a normal nt5e gene of the bastard halibut is a gene that includes a polynucleotide (Pp) below:
(Pp) any of polynucleotides (Pp 1) to (Pp7) below:
(Pp1) a polynucleotide having a base sequence of SEQ ID NO: 11;
(Pp2) a polynucleotide that encodes a protein having an activity of degrading inosinic acid and that has a base sequence consisting of the base sequence of (Pp1) above with a deletion, a substitution, an insertion, and/or an addition of one or several bases;
(Pp3) a polynucleotide that encodes a protein having an activity of degrading inosinic acid and that has a base sequence having 90% or more identity to the base sequence of (Pp1) above;
(Pp4) a polynucleotide that encodes a protein having an activity of degrading inosinic acid and that has a base sequence complementary to a polynucleotide capable of hybridizing to the polynucleotide having the base sequence of (Pp1) above under a stringent condition;
(Pp5) a polynucleotide encoding a protein having an amino acid sequence of SEQ ID NO: 12;
(Pp6) a polynucleotide encoding a protein that has an activity of degrading inosinic acid and that has an amino acid sequence consisting of the amino acid sequence of SEQ ID NO: 12 with a deletion, a substitution, an insertion, and/or an addition of one or several amino acids; and
(Pp7) a polynucleotide encoding a protein that has an activity of degrading inosinic acid and that has an amino acid sequence having 90% or more identity to the amino acid sequence of SEQ ID NO: 12.

### (Supplementary Note 20)

The fish according to any one of Supplementary Notes 2 to 6 and 19,

wherein the fish is a bastard halibut (Paralichthys olivaceus), and

the bastard halibut includes, as a loss-of-function nt5e gene, a mutant gene having mutation in exon 4 of a normal gene of an nt5e gene.

### (Supplementary Note 21)

The fish according to Supplementary Note 20,

wherein the bastard halibut includes, as a loss-of-function nt5e gene, a mutant gene that has mutation in the exon 4 of the normal nt5e gene and that has a base sequence consisting of a base sequence of the normal nt5e gene with frameshift mutation or nonsense mutation.

### (Supplementary Note 22)

The fish according to any one of Supplementary Notes 2 to 6 and 19 to 21,

wherein the fish is a bastard halibut (Paralichthys olivaceus), and

the bastard halibut has, as a loss-of-function nt5e gene, mutation in bases from position 605 to position 627 of a base sequence of SEQ ID NO: 11.

### (Supplementary Note 23)

The fish according to any one of Supplementary Notes 2 to 6,

wherein the fish is a whitespotted clarias (Clarias garienpinus), and

a normal nt5e gene of the whitespotted clarias is a gene that includes a polynucleotide (Pq) below:
(Pq) any of polynucleotides (Pq1) to (Pq7) below:
(Pq1) a polynucleotide having a base sequence of SEQ ID NO: 25;
(Pq2) a polynucleotide that encodes a protein having an activity of degrading inosinic acid and that has a base sequence consisting of the base sequence of (Pq1) above with a deletion, a substitution, an insertion, and/or an addition of one or several bases;
(Pq3) a polynucleotide that encodes a protein having an activity of degrading inosinic acid and that has a base sequence having 90% or more identity to the base sequence of (Pq1) above;
(Pq4) a polynucleotide that encodes a protein having an activity of degrading inosinic acid and that has a base sequence complementary to a polynucleotide capable of hybridizing to the polynucleotide having the base sequence of (Pq1) above under a stringent condition;
(Pq5) a polynucleotide encoding a protein having an amino acid sequence of SEQ ID NO: 26;
(Pq6) a polynucleotide encoding a protein that has an activity of degrading inosinic acid and that has an amino acid sequence consisting of the amino acid sequence of SEQ ID NO: 26 with a deletion, a substitution, an insertion, and/or an addition of one or several amino acids; and
(Pq7) a polynucleotide encoding a protein that has an activity of degrading inosinic acid and that has an amino acid sequence having 90% or more identity to the amino acid sequence of SEQ ID NO: 26.

### (Supplementary Note 24)

The fish according to any one of Supplementary Notes 2 to 6, 8 to 10, 12 to 14, 16 to 18, and 20 to 22,

wherein the loss-of-function nt5e gene encodes a mutant NT5E protein with reduced an activity of degrading inosinic acid.

### (Supplementary Note 25)

The fish according to any one of Supplementary Notes 1 to 6,

wherein the fish is a fish selected from the group consisting of the family Tetraodontidae, the family Sparidae, the family Salmonidae, the family Cyprinidae, the family Serranidae, the family Paralichthyidae, the family Clariidae, and the family Cichlidae.

### <Portion of Fish>

### (Supplementary Note 26)

A portion of a fish, the fish being defined in any one of Supplementary Notes 1 to 25.

### (Supplementary Note 27)

The portion of a fish according to Supplementary Note 26,

wherein the portion is an edible portion.

### <Fish Production Method>

### (Supplementary Note 28)

A fish production method comprising a step (a) below:
(a) a mating step of mating the fish according to any one of Supplementary Notes 1 to 25 with another fish.

### (Supplementary Note 29)

The production method according to Supplementary Note 28, comprising a step (b) below:
(b) a selection step of selecting a fish in which a function of an ecto-5′-nucleotidase (nt5e) gene is lost from fish obtained in the step (a) or a progeny line thereof.

### (Supplementary Note 30)

The production method according to Supplementary Note 28 or 29, comprising a step (x) below performed prior to the step (a):
(x) a step of selecting the fish according to any one of Supplementary Notes 1 to 25 from target fish.

### (Supplementary Note 31)

The production method according to Supplementary Note 30,

wherein selection in the step (x) is selection of a fish that includes a loss-of-function ecto-5′-nucleotidase (nt5e) gene.

### (Supplementary Note 32)

The production method according to Supplementary Note 28 or 29, comprising a step (y) below performed prior to the step (a):
(y) a breeding step of breeding the fish according to any one of Supplementary Notes 1 to 25 from target fish.

### <Method for Producing Accelerated Ripened Fish>

### (Supplementary Note 33)

A method for producing an accelerated ripened fish, comprising a functional loss-causing step of causing loss of a function of an ecto-5′-nucleotidase (nt5e) gene of a target fish.

### (Supplementary Note 34)

The production method according to Supplementary Note 33,

wherein, in the functional loss causing step, a fish that includes a loss-of-function nt5e gene is bred by introducing loss-of-function mutation into the nt5e gene of the target fish.

### (Supplementary Note 35)

The production method according to Supplementary Note 34,

wherein the loss-of-function nt5e gene is a mutant gene having a base sequence consisting of a base sequence of a normal nt5e gene with a deletion, a substitution, an insertion, and/or an addition of one or several bases.

### (Supplementary Note 36)

The production method according to Supplementary Note 35,

wherein the loss-of-function nt5e gene is a mutant gene having a base sequence consisting of the base sequence of the normal nt5e gene with deletion of at least some bases.

### (Supplementary Note 37)

The production method according to any one of Supplementary Notes 34 to 36,

wherein the loss-of-function nt5e gene is a mutant gene having a base sequence consisting of a base sequence of a normal nt5e gene with frameshift mutation.

### (Supplementary Note 38)

The production method according to any one of Supplementary Notes 34 to 37,

wherein the loss-of-function nt5e gene is a mutant gene having a base sequence consisting of a base sequence of a normal nt5e gene with nonsense mutation.

### (Supplementary Note 39)

The production method according to any one of Supplementary Notes 34 to 38,

wherein the loss-of-function nt5e gene is a mutant gene having a base sequence consisting of a base sequence of a normal nt5e gene with mutation in a first exon.

### (Supplementary Note 40)

The production method according to any one of Supplementary Notes 34 to 39,

wherein the fish is a red seabream (Pagrus major), and

a normal nt5e gene of the red seabream is a gene that includes a polynucleotide (Pn) below:
(Pn) any of polynucleotides (Pn1) to (Pn7) below:
(Pn1) a polynucleotide having any base sequence of SEQ ID NO: 1;
(Pn2) a polynucleotide that encodes a protein having an activity of degrading inosinic acid and that has a base sequence consisting of the base sequence of (Pn1) above with a deletion, a substitution, an insertion, and/or an addition of one or several bases;
(Pn3) a polynucleotide that encodes a protein having an activity of degrading inosinic acid and that has a base sequence having 80% or more identity to the base sequence of (Pn1) above;
(Pn4) a polynucleotide that encodes a protein having an activity of degrading inosinic acid and that has a base sequence complementary to a polynucleotide capable of hybridizing to the polynucleotide having the base sequence of (Pn1) above under a stringent condition;
(Pn5) a polynucleotide encoding a protein having an amino acid sequence of SEQ ID NO: 2;
(Pn6) a polynucleotide encoding a protein that has an activity of degrading inosinic acid and that has an amino acid sequence consisting of the amino acid sequence of SEQ ID NO: 2 with a deletion, a substitution, an insertion, and/or an addition of one or several amino acids; and
(Pn7) a polynucleotide encoding a protein that has an activity of degrading inosinic acid and that has an amino acid sequence having 80% or more identity to the amino acid sequence of SEQ ID NO: 2.

### (Supplementary Note 41)

The production method according to any one of Supplementary Notes 34 to 40,

wherein the fish is a red seabream (Pagrus major), and

the red seabream includes, as a loss-of-function nt5e gene, a mutant gene having mutation in exon 6 of a normal nt5e gene.

### (Supplementary Note 42)

The production method according to Supplementary Note 41,

wherein the red seabream includes, as a loss-of-function nt5e gene, a mutant gene that has mutation in the exon 6 of the normal nt5e gene and that has a base sequence consisting of a base sequence of the normal nt5e gene with frameshift mutation or nonsense mutation.

### (Supplementary Note 43)

The production method according to any one of Supplementary Notes 34 to 42,

wherein the fish is a red seabream (Pagrus major), and

the red seabream includes, as a loss-of-function nt5e gene, a mutant gene having mutation in bases from position 1014 to position 1036 of a base sequence of SEQ ID NO: 1.

### (Supplementary Note 44)

The production method according to any one of Supplementary Notes 34 to 39,

wherein the fish is a tiger puffer (Takifugu rubripes), and

a normal nt5e gene of the tiger puffer is a gene that includes a polynucleotide (Pt) below:
(Pt) any of polynucleotides (Pt1) to (Pt7) below:
(Pt1) a polynucleotide having any base sequence of SEQ ID NO: 3;
(Pt2) a polynucleotide that encodes a protein having an activity of degrading inosinic acid and that has a base sequence consisting of the base sequence of (Pt1) above with a deletion, a substitution, an insertion, and/or an addition of one or several bases;
(Pt3) a polynucleotide that encodes a protein having an activity of degrading inosinic acid and that has a base sequence having 80% or more identity to the base sequence of (Pt1) above;
(Pt4) a polynucleotide that encodes a protein having an activity of degrading inosinic acid and that has a base sequence complementary to a polynucleotide capable of hybridizing to the polynucleotide having the base sequence of (Pt1) above under a stringent condition;
(Pt5) a polynucleotide encoding a protein having an amino acid sequence of SEQ ID NO: 4;
(Pt6) a polynucleotide encoding a protein that has an activity of degrading inosinic acid and that has an amino acid sequence consisting of the amino acid sequence of SEQ ID NO: 4 with a deletion, a substitution, an insertion, and/or an addition of one or several amino acids; and
(Pt7) a polynucleotide encoding a protein that has an activity of degrading inosinic acid and that has an amino acid sequence having 80% or more identity to the amino acid sequence of SEQ ID NO: 4.

### (Supplementary Note 45)

The production method according to any one of Supplementary Notes 34 to 39 and 44,

wherein the fish is a tiger puffer (Takifugu rubripes), and

the tiger puffer includes, as a loss-of-function nt5e gene, a mutant gene having mutation in exon 1 of a normal gene of a normal nt5e gene.

### (Supplementary Note 46)

The production method according to Supplementary Note 45,

wherein the tiger puffer includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 47)

The production method according to any one of Supplementary Notes 34 to 39 and 44 to 46,

wherein the fish is a tiger puffer (Takifugu rubripes), and

the tiger puffer has, as a loss-of-function nt5e gene, mutation in bases from position 131 to position 153 of a base sequence of SEQ ID NO: 3.

### (Supplementary Note 43)

The production method according to any one of Supplementary Notes 34 to 39,

wherein the fish is a tilapia (Oreochromis niloticus), and

a normal nt5e gene of the tilapia is a gene that includes a polynucleotide (Po) below:
(Po) any of polynucleotides (Po1) to (Po7) below:
(Po1) a polynucleotide having any base sequence of SEQ ID NO: 5;
(Po2) a polynucleotide that encodes a protein having an activity of degrading inosinic acid and that has a base sequence consisting of the base sequence of (Po1) above with a deletion, a substitution, an insertion, and/or an addition of one or several bases;
(Po3) a polynucleotide that encodes a protein having an activity of degrading inosinic acid and that has a base sequence having 80% or more identity to the base sequence of (Po1) above;
(Po4) a polynucleotide that encodes a protein having an activity of degrading inosinic acid and that has a base sequence complementary to a polynucleotide capable of hybridizing to the polynucleotide having the base sequence of (Po1) above under a stringent condition;
(Po5) a polynucleotide encoding a protein having an amino acid sequence of SEQ ID NO: 6;
(Po6) a polynucleotide encoding a protein that has an activity of degrading inosinic acid and that has an amino acid sequence consisting of the amino acid sequence of SEQ ID NO: 6 with a deletion, a substitution, an insertion, and/or an addition of one or several amino acids; and
(Po7) a polynucleotide encoding a protein that has an activity of degrading inosinic acid and that has an amino acid sequence having 80% or more identity to the amino acid sequence of SEQ ID NO: 6.

### (Supplementary Note 49)

The production method according to any one of Supplementary Notes 34 to 39 and 48,

wherein the fish is a tilapia (Oreochromis niloticus), and

the tilapia includes, as a loss-of-function nt5e gene, a mutant gene having mutation in exon 1 of a normal gene of an nt5e gene.

### (Supplementary Note 50)

The production method according to Supplementary Note 49,

wherein the tilapia includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 51)

The fish according to any one of Supplementary Notes 34 to 39 and 48 to 50,

wherein the fish is a tilapia (Oreochromis niloticus), and

the tilapia has, as a loss-of-function nt5e gene, mutation in bases from position 217 to position 239 of a base sequence of SEQ ID NO: 5.

### (Supplementary Note 52)

The fish according to any one of Supplementary Notes 34 to 39,

wherein the fish is a bastard halibut (Paralichthys olivaceus), and

a normal nt5e gene of the bastard halibut is a gene that includes a polynucleotide (Pp) below:
(Pp) any of polynucleotides (Pp1) to (Pp7) below:
(Pp1) a polynucleotide having a base sequence of SEQ ID NO: 11;
(Pp2) a polynucleotide that encodes a protein having an activity of degrading inosinic acid and that has a base sequence consisting of the base sequence of (Pp1) above with a deletion, a substitution, an insertion, and/or an addition of one or several bases;
(Pp3) a polynucleotide that encodes a protein having an activity of degrading inosinic acid and that has a base sequence having 90% or more identity to the base sequence of (Pp1) above;
(Pp4) a polynucleotide that encodes a protein having an activity of degrading inosinic acid and that has a base sequence complementary to a polynucleotide capable of hybridizing to the polynucleotide having the base sequence of (Pp1) above under a stringent condition;
(Pp5) a polynucleotide encoding a protein having an amino acid sequence of SEQ ID NO: 12;
(Pp6) a polynucleotide encoding a protein that has an activity of degrading inosinic acid and that has an amino acid sequence consisting of the amino acid sequence of SEQ ID NO: 12 with a deletion, a substitution, an insertion, and/or an addition of one or several amino acids; and
(Pp7) a polynucleotide encoding a protein that has an activity of degrading inosinic acid and that has an amino acid sequence having 90% or more identity to the amino acid sequence of SEQ ID NO: 12.

### (Supplementary Note 53)

The fish according to any one of Supplementary Notes 34 to 39 and 52,

wherein the fish is a bastard halibut (Paralichthys olivaceus), and

the bastard halibut includes, as a loss-of-function nt5e gene, a mutant gene having mutation in exon 4 of a normal gene of an nt5e gene.

### (Supplementary Note 54)

The fish according to Supplementary Note 53,

wherein the bastard halibut includes, as a loss-of-function nt5e gene, a mutant gene that has mutation in the exon 4 of the normal nt5e gene and that has a base sequence consisting of a base sequence of the normal nt5e gene with frameshift mutation or nonsense mutation.

### (Supplementary Note 55)

The fish according to any one of Supplementary Notes 34 to 39 and 52 to 54,

wherein the fish is a bastard halibut (Paralichthys olivaceus), and

the bastard halibut has, as a loss-of-function nt5e gene, mutation in bases from position 605 to position 627 of a base sequence of SEQ ID NO: 11.

### (Supplementary Note 56)

The fish according to any one of claims 34 to 39,

wherein the fish is a whitespotted clarias (Clarias garienpinus), and

a normal nt5e gene of the whitespotted clarias is a gene that includes a polynucleotide (Pq) below:
(Pq) any of polynucleotides (Pq1) to (Pq7) below:
(Pq1) a polynucleotide having a base sequence of SEQ ID NO: 25;
(Pq2) a polynucleotide that encodes a protein having an activity of degrading inosinic acid and that has a base sequence consisting of the base sequence of (Pq1) above with a deletion, a substitution, an insertion, and/or an addition of one or several bases;
(Pq3) a polynucleotide that encodes a protein having an activity of degrading inosinic acid and that has a base sequence having 90% or more identity to the base sequence of (Pq1) above;
(Pq4) a polynucleotide that encodes a protein having an activity of degrading inosinic acid and that has a base sequence complementary to a polynucleotide capable of hybridizing to the polynucleotide having the base sequence of (Pq1) above under a stringent condition;
(Pq5) a polynucleotide encoding a protein having an amino acid sequence of SEQ ID NO: 26;
(Pq6) a polynucleotide encoding a protein that has an activity of degrading inosinic acid and that has an amino acid sequence consisting of the amino acid sequence of SEQ ID NO: 26 with a deletion, a substitution, an insertion, and/or an addition of one or several amino acids; and
(Pq7) a polynucleotide encoding a protein that has an activity of degrading inosinic acid and that has an amino acid sequence having 90% or more identity to the amino acid sequence of SEQ ID NO: 26.

### (Supplementary Note 57)

The production method according to any one of Supplementary Notes 24 to 29, 41 to 43, 45 to 47, 49 to 51, and 53 to 55,

wherein the loss-of-function nt5e gene encodes a mutant NT5E protein with a reduced activity of degrading inosinic acid.

### (Supplementary Note 58)

The production method according to any one of Supplementary Notes 34 to 57,

wherein the breeding step includes:
an introduction step of introducing mutation into an nt5e gene of target fish; and
a selection step of selecting, as an accelerated ripened fish, a fish that includes a loss-of-function nt5e gene from the target fish into which the mutation has been introduced.

### <Method for Enhancing Inosinic Acid Content During Ripening of Fish Meat>

### (Supplementary Note 59)

A method for enhancing an inosinic acid content during ripening of fish meat, comprising a ripening step of ripening fish meat,

wherein the fish meat is fish meat of the fish according to any one of Supplementary Notes 1 to 25, and/or fish meat in the edible portion of a fish according to Supplementary Note 26 or 27.

### (Supplementary Note 60)

The enhancing method according to Supplementary Note 59,

wherein the fish meat is ripened for 1 to 31 days in the ripening step.

### <Method for Screening Accelerated Ripened Fish>

### (Supplementary Note 61)

A method for screening an accelerated ripened fish, comprising a selection step of selecting, from test fish, a test fish in which a function of an ecto-5′-nucleotidase (nt5e) gene is lost as an accelerated ripened fish.

### (Supplementary Note 62)

The screening method according to Supplementary Note 61,

wherein a test fish that includes a loss-of-function nt5e gene is selected as an accelerated ripened fish in the selection step, and

the loss-of-function nt5e gene is a mutant gene having a base sequence consisting of a base sequence of a normal nt5e gene with a deletion, a substitution, an insertion, and/or an addition of one or several bases.

### (Supplementary Note 63)

The screening method according to Supplementary Note 62,

wherein the loss-of-function nt5e gene is a mutant gene having a base sequence consisting of the base sequence of the normal nt5e gene with deletion of at least some bases.

### (Supplementary Note 64)

The screening method according to Supplementary Note 62 or 63,

wherein a test fish that includes a loss-of-function nt5e gene is selected as an accelerated ripened fish in the selection step, and

the loss-of-function nt5e gene is a mutant gene having a base sequence consisting of a base sequence of a normal nt5e gene with frameshift mutation.

### (Supplementary Note 65)

The screening method according to any one of Supplementary Notes 62 to 64,

wherein a test fish that includes a loss-of-function nt5e gene is selected as an accelerated ripened fish in the selection step, and

the loss-of-function nt5e gene is a mutant gene having a base sequence consisting of a base sequence of a normal nt5e gene with nonsense mutation.

### (Supplementary Note 66)

The screening method according to any one of Supplementary Notes 62 to 65,

wherein a test fish that includes a loss-of-function nt5e gene is selected as an accelerated ripened fish in the selection step, and

the loss-of-function nt5e gene is a mutant gene having a base sequence consisting of a base sequence of a normal nt5e gene with mutation in a first exon.

### (Supplementary Note 67)

The screening method according to any one of Supplementary Notes 62 to 66,

wherein the fish is a red seabream (Pagrus major), and

a normal nt5e gene of the red seabream is a gene that includes a polynucleotide (Pn) below:
(Pn) any of polynucleotides (Pn1) to (Pn7) below:
(Pn1) a polynucleotide having any base sequence of SEQ ID NO: 1;
(Pn2) a polynucleotide that encodes a protein having an activity of degrading inosinic acid and that has a base sequence consisting of the base sequence of (Pn1) above with a deletion, a substitution, an insertion, and/or an addition of one or several bases;
(Pn3) a polynucleotide that encodes a protein having an activity of degrading inosinic acid and that has a base sequence having 80% or more identity to the base sequence of (Pn1) above;
(Pn4) a polynucleotide that encodes a protein having an activity of degrading inosinic acid and that has a base sequence complementary to a polynucleotide capable of hybridizing to the polynucleotide having the base sequence of (Pn1) above under a stringent condition;
(Pn5) a polynucleotide encoding a protein having an amino acid sequence of SEQ ID NO: 2;
(Pn6) a polynucleotide encoding a protein that has an activity of degrading inosinic acid and that has an amino acid sequence consisting of the amino acid sequence of SEQ ID NO: 2 with a deletion, a substitution, an insertion, and/or an addition of one or several amino acids; and
(Pn7) a polynucleotide encoding a protein that has an activity of degrading inosinic acid and that has an amino acid sequence having 80% or more identity to the amino acid sequence of SEQ ID NO: 2.

### (Supplementary Note 68)

The screening method according to any one of Supplementary Notes 62 to 67,

wherein the fish is a red seabream (Pagrus major), and

the red seabream includes, as a loss-of-function nt5e gene, a mutant gene having mutation in exon 6 of a normal nt5e gene.

### (Supplementary Note 69)

The screening method according to Supplementary Note 68,

wherein the red seabream includes, as a loss-of-function nt5e gene, a mutant gene that has mutation in the exon 6 of the normal nt5e gene and that has a base sequence consisting of a base sequence of the normal nt5e gene with frameshift mutation or nonsense mutation.

### (Supplementary Note 70)

The screening method according to any one of Supplementary Notes 62 to 69,

wherein the fish is a red seabream (Pagrus major), and

the red seabream includes, as a loss-of-function nt5e gene, a mutant gene having mutation in bases from position 1014 to position 1036 of a base sequence of SEQ ID NO: 1.

### (Supplementary Note 71)

The screening method according to any one of Supplementary Notes 62 to 66,

wherein the fish is a tiger puffer (Takifugu rubripes), and

a normal nt5e gene of the tiger puffer is a gene that includes a polynucleotide (Pt) below:
(Pt) any of polynucleotides (Pt1) to (Pt7) below:
(Pt1) a polynucleotide having any base sequence of SEQ ID NO: 3;
(Pt2) a polynucleotide that encodes a protein having an activity of degrading inosinic acid and that has a base sequence consisting of the base sequence of (Pt1) above with a deletion, a substitution, an insertion, and/or an addition of one or several bases;
(Pt3) a polynucleotide that encodes a protein having an activity of degrading inosinic acid and that has a base sequence having 80% or more identity to the base sequence of (Pt1) above;
(Pt4) a polynucleotide that encodes a protein having an activity of degrading inosinic acid and that has a base sequence complementary to a polynucleotide capable of hybridizing to the polynucleotide having the base sequence of (Pt1) above under a stringent condition;
(Pt5) a polynucleotide encoding a protein having an amino acid sequence of SEQ ID NO: 4;
(Pt6) a polynucleotide encoding a protein that has an activity of degrading inosinic acid and that has an amino acid sequence consisting of the amino acid sequence of SEQ ID NO: 4 with a deletion, a substitution, an insertion, and/or an addition of one or several amino acids; and
(Pt7) a polynucleotide encoding a protein that has an activity of degrading inosinic acid and that has an amino acid sequence having 80% or more identity to the amino acid sequence of SEQ ID NO: 4.

### (Supplementary Note 72)

The screening method according to any one of Supplementary Notes 62 to 66 and 71,

wherein the fish is a tiger puffer (Takifugu rubripes), and

the tiger puffer includes, as a loss-of-function nt5e gene, a mutant gene having mutation in exon 1 of a normal gene of a normal nt5e gene.

### (Supplementary Note 73)

The screening method according to Supplementary Note 72,

wherein the tiger puffer includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 74)

The screening method according to any one of Supplementary Notes 62 to 66 and 71 to 73,

wherein the fish is a tiger puffer (Takifugu rubripes), and

the tiger puffer has, as a loss-of-function nt5e gene, mutation in bases from position 131 to position 153 of a base sequence of SEQ ID NO: 3.

### (Supplementary Note 75)

The screening method according to any one of Supplementary Notes 62 to 66,

wherein the fish is a tilapia (Oreochromis niloticus), and

a normal nt5e gene of the tilapia is a gene that includes a polynucleotide (Po) below:
(Po) any of polynucleotides (Po1) to (Po7) below:
(Po1) a polynucleotide having any base sequence of SEQ ID NO: 5;
(Po2) a polynucleotide that encodes a protein having an activity of degrading inosinic acid and that has a base sequence consisting of the base sequence of (Po1) above with a deletion, a substitution, an insertion, and/or an addition of one or several bases;
(Po3) a polynucleotide that encodes a protein having an activity of degrading inosinic acid and that has a base sequence having 80% or more identity to the base sequence of (Po1) above;
(Po4) a polynucleotide that encodes a protein having an activity of degrading inosinic acid and that has a base sequence complementary to a polynucleotide capable of hybridizing to the polynucleotide having the base sequence of (Po1) above under a stringent condition;
(Po5) a polynucleotide encoding a protein having an amino acid sequence of SEQ ID NO: 6;
(Po6) a polynucleotide encoding a protein that has an activity of degrading inosinic acid and that has an amino acid sequence consisting of the amino acid sequence of SEQ ID NO: 6 with a deletion, a substitution, an insertion, and/or an addition of one or several amino acids; and
(Po7) a polynucleotide encoding a protein that has an activity of degrading inosinic acid and that has an amino acid sequence having 80% or more identity to the amino acid sequence of SEQ ID NO: 6.

### (Supplementary Note 76)

The screening method according to any one of Supplementary Notes 62 to 66 and 75,

wherein the fish is a tilapia (Oreochromis niloticus), and

the tilapia includes, as a loss-of-function nt5e gene, a mutant gene having mutation in exon 1 of a normal gene of an nt5e gene.

### (Supplementary Note 77)

The screening method according to Supplementary Note 76,

wherein the tilapia includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 78)

The screening method according to any one of Supplementary Notes 62 to 66 and 75 to 77,

wherein the fish is a tilapia (Oreochromis niloticus), and

the tilapia has, as a loss-of-function nt5e gene, mutation in bases from position 217 to position 239 of a base sequence of SEQ ID NO: 5.

### (Supplementary Note 79)

The screening method according to any one of Supplementary Notes 62 to 66,

wherein the fish is a bastard halibut (Paralichthys olivaceus), and

a normal nt5e gene of the bastard halibut is a gene that includes a polynucleotide (Pp) below:
(Pp) any of polynucleotides (Pp1) to (Pp7) below:
(Pp1) a polynucleotide having any base sequence of SEQ ID NO: 11;
(Pp2) a polynucleotide that encodes a protein having an activity of degrading inosinic acid and that has a base sequence consisting of the base sequence of (Pp1) above with a deletion, a substitution, an insertion, and/or an addition of one or several bases;
(Pp3) a polynucleotide that encodes a protein having an activity of degrading inosinic acid and that has a base sequence having 80% or more identity to the base sequence of (Pp1) above;
(Pp4) a polynucleotide that encodes a protein having an activity of degrading inosinic acid and that has a base sequence complementary to a polynucleotide capable of hybridizing to the polynucleotide having the base sequence of (Pp1) above under a stringent condition;
(Pp5) a polynucleotide encoding a protein having an amino acid sequence of SEQ ID NO: 12;
(Pp6) a polynucleotide encoding a protein that has an activity of degrading inosinic acid and that has an amino acid sequence consisting of the amino acid sequence of SEQ ID NO: 12 with a deletion, a substitution, an insertion, and/or an addition of one or several amino acids; and
(Pp7) a polynucleotide encoding a protein that has an activity of degrading inosinic acid and that has an amino acid sequence having 80% or more identity to the amino acid sequence of SEQ ID NO: 12.

### (Supplementary Note 80)

The screening method according to any one of Supplementary Notes 62 to 66 and 79,

wherein the fish is a bastard halibut (Paralichthys olivaceus), and

the bastard halibut includes, as a loss-of-function nt5e gene, a mutant gene having mutation in exon 4 of a normal gene of an nt5e gene.

### (Supplementary Note 81)

The screening method according to Supplementary Note 80,

wherein the bastard halibut includes, as a loss-of-function nt5e gene, a mutant gene that has mutation in the exon 4 of the normal nt5e gene and that has a base sequence consisting of a base sequence of the normal nt5e gene with frameshift mutation or nonsense mutation.

### (Supplementary Note 82)

The screening method according to any one of Supplementary Notes 62 to 66 and 79 to 81,

wherein the fish is a bastard halibut (Paralichthys olivaceus), and

the bastard halibut has, as a loss-of-function nt5e gene, mutation in bases from position 605 to position 627 of a base sequence of SEQ ID NO: 11.

### (Supplementary Note 83)

The screening method according to any one of Supplementary Notes 62 to 66,

wherein the fish is a whitespotted clarias (Clarias garienpinus), and

a normal nt5e gene of the whitespotted clarias is a gene that includes a polynucleotide (Pq) below:
(Pq) any of polynucleotides (Pq1) to (Pq7) below:
(Pq1) a polynucleotide having a base sequence of SEQ ID NO: 25;
(Pq2) a polynucleotide that encodes a protein having an activity of degrading inosinic acid and that has a base sequence consisting of the base sequence of (Pq1) above with a deletion, a substitution, an insertion, and/or an addition of one or several bases;
(Pq3) a polynucleotide that encodes a protein having an activity of degrading inosinic acid and that has a base sequence having 90% or more identity to the base sequence of (Pq1) above;
(Pq4) a polynucleotide that encodes a protein having an activity of degrading inosinic acid and that has a base sequence complementary to a polynucleotide capable of hybridizing to the polynucleotide having the base sequence of (Pq1) above under a stringent condition;
(Pq5) a polynucleotide encoding a protein having an amino acid sequence of SEQ ID NO: 26;
(Pq6) a polynucleotide encoding a protein that has an activity of degrading inosinic acid and that has an amino acid sequence consisting of the amino acid sequence of SEQ ID NO: 26 with a deletion, a substitution, an insertion, and/or an addition of one or several amino acids; and
(Pq7) a polynucleotide encoding a protein that has an activity of degrading inosinic acid and that has an amino acid sequence having 90% or more identity to the amino acid sequence of SEQ ID NO: 26.

### <Fish Production Method>

### (Supplementary Note 84)

A fish production method comprising a screening step of screening, from test fish, a test fish in which a function of an ecto-5′-nucleotidase (nt5e) gene is lost,

wherein the screening step is conducted in accordance with the screening method according to any one of Supplementary Notes 62 to 78.

### <Fish>

### (Supplementary Note 85)

A fish obtained through the production method according to any one of Supplementary Notes 28 to 58 and 84.

### <Detection Method>

### (Supplementary Note 86)

A method for detecting an ability of a fish to be ripened with acceleration, comprising a detection step of detecting if a function of an ecto-5′-nucleotidase (nt5e) gene is lost in a test fish.

### (Supplementary Note 87)

The detection method according to Supplementary Note 86,

wherein the detection step includes a detection step of detecting if a loss-of-function nt5e gene is present in a test fish.

### (Supplementary Note 88)

The detection method according to Supplementary Note 87,

wherein the loss-of-function nt5e gene is a mutant gene having a base sequence consisting of a base sequence of a normal nt5e gene with a deletion, a substitution, an insertion, and/or an addition of one or several bases.

### (Supplementary Note 89)

The detection method according to Supplementary Note 88,

wherein the loss-of-function nt5e gene is a mutant gene having a base sequence consisting of the base sequence of the normal nt5e gene with deletion of at least some bases.

### (Supplementary Note 90)

The detection method according to any one of Supplementary Notes 87 to 89,

wherein the loss-of-function nt5e gene is a mutant gene having a base sequence consisting of a base sequence of a normal nt5e gene with frameshift mutation.

### (Supplementary Note 91)

The detection method according to any one of Supplementary Notes 87 to 90,

wherein the loss-of-function nt5e gene is a mutant gene having a base sequence consisting of a base sequence of a normal nt5e gene with nonsense mutation.

### (Supplementary Note 92)

The detection method according to any one of Supplementary Notes 87 to 91,

wherein the loss-of-function nt5e gene is a mutant gene having a base sequence consisting of a base sequence of a normal nt5e gene with mutation in a first exon.

### (Supplementary Note 93)

The detection method according to any one of Supplementary Notes 87 to 92,

wherein the fish is a red seabream (Pagrus major), and

a normal nt5e gene of the red seabream is a gene that includes a polynucleotide (Pn) below:
(Pn) any of polynucleotides (Pn1) to (Pn7) below:
(Pn1) a polynucleotide having any base sequence of SEQ ID NO: 1;
(Pn2) a polynucleotide that encodes a protein having an activity of degrading inosinic acid and that has a base sequence consisting of the base sequence of (Pn1) above with a deletion, a substitution, an insertion, and/or an addition of one or several bases;
(Pn3) a polynucleotide that encodes a protein having an activity of degrading inosinic acid and that has a base sequence having 80% or more identity to the base sequence of (Pn1) above;
(Pn4) a polynucleotide that encodes a protein having an activity of degrading inosinic acid and that has a base sequence complementary to a polynucleotide capable of hybridizing to the polynucleotide having the base sequence of (Pn1) above under a stringent condition;
(Pn5) a polynucleotide encoding a protein having an amino acid sequence of SEQ ID NO: 2;
(Pn6) a polynucleotide encoding a protein that has an activity of degrading inosinic acid and that has an amino acid sequence consisting of the amino acid sequence of SEQ ID NO: 2 with a deletion, a substitution, an insertion, and/or an addition of one or several amino acids; and
(Pn7) a polynucleotide encoding a protein that has an activity of degrading inosinic acid and that has an amino acid sequence having 80% or more identity to the amino acid sequence of SEQ ID NO: 2.

### (Supplementary Note 94)

The detection method according to any one of Supplementary Notes 87 to 93,

wherein the fish is a red seabream (Pagrus major), and

the red seabream includes, as a loss-of-function nt5e gene, a mutant gene having mutation in exon 6 of a normal nt5e gene.

### (Supplementary Note 95)

The detection method according to Supplementary Note 94,

wherein the red seabream includes, as a loss-of-function nt5e gene, a mutant gene that has mutation in the exon 6 of the normal nt5e gene and that has a base sequence consisting of a base sequence of the normal nt5e gene with frameshift mutation or nonsense mutation.

### (Supplementary Note 96)

The detection method according to any one of Supplementary Notes 87 to 95,

wherein the fish is a red seabream (Pagrus major), and

the red seabream includes, as a loss-of-function nt5e gene, a mutant gene having mutation in bases from position 1014 to position 1036 of a base sequence of SEQ ID NO: 1.

### (Supplementary Note 97)

The detection method according to any one of Supplementary Notes 87 to 92,

wherein the fish is a tiger puffer (Takifugu rubripes), and

a normal nt5e gene of the tiger puffer is a gene that includes a polynucleotide (Pt) below:
(Pt) any of polynucleotides (Pt1) to (Pt7) below:
(Pt1) a polynucleotide having any base sequence of SEQ ID NO: 3;
(Pt2) a polynucleotide that encodes a protein having an activity of degrading inosinic acid and that has a base sequence consisting of the base sequence of (Pt1) above with a deletion, a substitution, an insertion, and/or an addition of one or several bases;
(Pt3) a polynucleotide that encodes a protein having an activity of degrading inosinic acid and that has a base sequence having 80% or more identity to the base sequence of (Pt1) above;
(Pt4) a polynucleotide that encodes a protein having an activity of degrading inosinic acid and that has a base sequence complementary to a polynucleotide capable of hybridizing to the polynucleotide having the base sequence of (Pt1) above under a stringent condition;
(Pt5) a polynucleotide encoding a protein having an amino acid sequence of SEQ ID NO: 4;
(Pt6) a polynucleotide encoding a protein that has an activity of degrading inosinic acid and that has an amino acid sequence consisting of the amino acid sequence of SEQ ID NO: 4 with a deletion, a substitution, an insertion, and/or an addition of one or several amino acids; and
(Pt7) a polynucleotide encoding a protein that has an activity of degrading inosinic acid and that has an amino acid sequence having 80% or more identity to the amino acid sequence of SEQ ID NO: 4.

### (Supplementary Note 98)

The detection method according to any one of Supplementary Notes 87 to 92 and 97,

wherein the fish is a tiger puffer (Takifugu rubripes), and

the tiger puffer includes, as a loss-of-function nt5e gene, a mutant gene having mutation in exon 1 of a normal gene of a normal nt5e gene.

### (Supplementary Note 99)

The detection method according to Supplementary Note 98,

wherein the tiger puffer includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 100)

The detection method according to any one of Supplementary Notes 87 to 92 and 97 to 99,

wherein the fish is a tiger puffer (Takifugu rubripes), and

the tiger puffer has, as a loss-of-function nt5e gene, mutation in bases from position 131 to position 153 of a base sequence of SEQ ID NO: 3.

### (Supplementary Note 101)

The detection method according to any one of Supplementary Notes 87 to 92,

wherein the fish is a tilapia (Oreochromis niloticus), and

a normal nt5e gene of the tilapia is a gene that includes a polynucleotide (Po) below:
(Po) any of polynucleotides (Po1) to (Po7) below:
(Po1) a polynucleotide having any base sequence of SEQ ID NO: 5;
(Po2) a polynucleotide that encodes a protein having an activity of degrading inosinic acid and that has a base sequence consisting of the base sequence of (Po1) above with a deletion, a substitution, an insertion, and/or an addition of one or several bases;
(Po3) a polynucleotide that encodes a protein having an activity of degrading inosinic acid and that has a base sequence having 80% or more identity to the base sequence of (Po1) above;
(Po4) a polynucleotide that encodes a protein having an activity of degrading inosinic acid and that has a base sequence complementary to a polynucleotide capable of hybridizing to the polynucleotide having the base sequence of (Po1) above under a stringent condition;
(Po5) a polynucleotide encoding a protein having an amino acid sequence of SEQ ID NO: 6;
(Po6) a polynucleotide encoding a protein that has an activity of degrading inosinic acid and that has an amino acid sequence consisting of the amino acid sequence of SEQ ID NO: 6 with a deletion, a substitution, an insertion, and/or an addition of one or several amino acids; and
(Po7) a polynucleotide encoding a protein that has an activity of degrading inosinic acid and that has an amino acid sequence having 80% or more identity to the amino acid sequence of SEQ ID NO: 6.

### (Supplementary Note 102)

The detection method according to any one of Supplementary Notes 87 to 92 and 101,

wherein the fish is a tilapia (Oreochromis niloticus), and

the tilapia includes, as a loss-of-function nt5e gene, a mutant gene having mutation in exon 1 of a normal gene of an nt5e gene.

### (Supplementary Note 103)

The detection method according to Supplementary Note 102,

wherein the tilapia includes, as a loss-of-function nt5e gene, a mutant gene that has mutation in the exon 1 of the normal nt5e gene and that has a base sequence consisting of a base sequence of the normal nt5e gene with frameshift mutation or nonsense mutation.

### (Supplementary Note 104)

The detection method according to any one of Supplementary Notes 87 to 92 and 101 to 103,

wherein the fish is a tilapia (Oreochromis niloticus), and

the tilapia has, as a loss-of-function nt5e gene, mutation in bases from position 217 to position 239 of a base sequence of SEQ ID NO: 5.

### (Supplementary Note 105)

The detection method according to any one of Supplementary Notes 87 to 92,

wherein the fish is a bastard halibut (Paralichthys olivaceus), and

a normal nt5e gene of the bastard halibut is a gene that includes a polynucleotide (Pp) below:
(Pp) any of polynucleotides (Pp1) to (Pp7) below:
(Pp1) a polynucleotide having any base sequence of SEQ ID NO: 11;
(Pp2) a polynucleotide that encodes a protein having an activity of degrading inosinic acid and that has a base sequence consisting of the base sequence of (Pp1) above with a deletion, a substitution, an insertion, and/or an addition of one or several bases;
(Pp3) a polynucleotide that encodes a protein having an activity of degrading inosinic acid and that has a base sequence having 80% or more identity to the base sequence of (Pp1) above;
(Pp4) a polynucleotide that encodes a protein having an activity of degrading inosinic acid and that has a base sequence complementary to a polynucleotide capable of hybridizing to the polynucleotide having the base sequence of (Pp1) above under a stringent condition;
(Pp5) a polynucleotide encoding a protein having an amino acid sequence of SEQ ID NO: 12;
(Pp6) a polynucleotide encoding a protein that has an activity of degrading inosinic acid and that has an amino acid sequence consisting of the amino acid sequence of SEQ ID NO: 12 with a deletion, a substitution, an insertion, and/or an addition of one or several amino acids; and
(Pp7) a polynucleotide encoding a protein that has an activity of degrading inosinic acid and that has an amino acid sequence having 80% or more identity to the amino acid sequence of SEQ ID NO: 12.

### (Supplementary Note 106)

The detection method according to any one of Supplementary Notes 87 to 92 and 105,

wherein the fish is a bastard halibut (Paralichthys olivaceus), and

the bastard halibut includes, as a loss-of-function nt5e gene, a mutant gene having mutation in exon 4 of a normal gene of an nt5e gene.

### (Supplementary Note 107)

The detection method according to Supplementary Note 106,

wherein the bastard halibut includes, as a loss-of-function nt5e gene, a mutant gene that has mutation in the exon 4 of the normal nt5e gene and that has a base sequence consisting of a base sequence of the normal nt5e gene with frameshift mutation or nonsense mutation.

### (Supplementary Note 108)

The detection method according to any one of Supplementary Notes 87 to 92 and 105 to 107,

wherein the fish is a bastard halibut (Paralichthys olivaceus), and

the bastard halibut has, as a loss-of-function nt5e gene, mutation in bases from position 605 to position 627 of a base sequence of SEQ ID NO: 11.

### (Supplementary Note 109)

The detection method according to any one of Supplementary Notes 87 to 92,

wherein the fish is a whitespotted clarias (Clarias garienpinus), and

a normal nt5e gene of the whitespotted clarias is a gene that includes a polynucleotide (Pq) below:
(Pq) any of polynucleotides (Pq1) to (Pq7) below:
(Pq1) a polynucleotide having a base sequence of SEQ ID NO: 25;
(Pq2) a polynucleotide that encodes a protein having an activity of degrading inosinic acid and that has a base sequence consisting of the base sequence of (Pq1) above with a deletion, a substitution, an insertion, and/or an addition of one or several bases;
(Pq3) a polynucleotide that encodes a protein having an activity of degrading inosinic acid and that has a base sequence having 90% or more identity to the base sequence of (Pq1) above;
(Pq4) a polynucleotide that encodes a protein having an activity of degrading inosinic acid and that has a base sequence complementary to a polynucleotide capable of hybridizing to the polynucleotide having the base sequence of (Pq1) above under a stringent condition;
(Pq5) a polynucleotide encoding a protein having an amino acid sequence of SEQ ID NO: 26;
(Pq6) a polynucleotide encoding a protein that has an activity of degrading inosinic acid and that has an amino acid sequence consisting of the amino acid sequence of SEQ ID NO: 26 with a deletion, a substitution, an insertion, and/or an addition of one or several amino acids; and
(Pq7) a polynucleotide encoding a protein that has an activity of degrading inosinic acid and that has an amino acid sequence having 90% or more identity to the amino acid sequence of SEQ ID NO: 26.

### (Supplementary Note 110)

The detection method according to any one of Supplementary Notes 87 to 92, 94 to 96, 98 to 100, 102 to 104, and 106 to 108,

wherein the loss-of-function nt5e gene encodes a mutant NT5E protein with a reduced activity of degrading inosinic acid.

### <Processed Food>

### (Supplementary Note 111)

Fish processed food containing the fish according to any one of Supplementary Notes 1 to 25 and 85.

### INDUSTRIAL APPLICABILITY

As described above, the fish of the present disclosure is ripened with acceleration. Accordingly, the present invention is very useful, for example, in the fields of fish breeding, fishery industry, and the like.

### SEQUENCE LISTING

### P22122WO.xml

1. A genetically modified fish in which a function of an ecto-5′-nucleotidase (nt5e) gene is lost.

2. The genetically modified fish according to claim 1, comprising an inactivated nt5e gene,
wherein the inactivated nt5e gene is a mutant gene having a base sequence of a normal nt5e gene having at least one mutation selected from the group consisting of a deletion, a substitution, an insertion, and an addition of one or more bases.

3. The genetically modified fish according to claim 2,
wherein the inactivated nt5e gene is a mutant gene having a base sequence of the normal nt5e gene having a deletion of at least a plurality of bases.

4. The genetically modified fish according to claim 1, comprising an inactivated nt5e gene,
wherein the inactivated nt5e gene is a mutant gene having a base sequence of a normal nt5e gene with frameshift mutation.

5. The genetically modified fish according to claim 1, comprising an inactivated nt5e gene,
wherein the inactivated nt5e gene is a mutant gene having a base sequence of a normal nt5e gene with a nonsense mutation.

6. The genetically modified fish according to claim 1, comprising an inactivated nt5e gene,
wherein the inactivated nt5e gene is a gene having a base sequence of a normal nt5e gene with a mutation in a first exon.

7. The genetically modified fish according to claim 2,
wherein the fish is a red seabream, and
a normal nt5e gene of the red seabream is a gene that comprises a polynucleotide (Pn) below:
(Pn) any polynucleotide selected from the group consisting of polynucleotides (Pn1) to (Pn7):
(Pn1) a polynucleotide having a base sequence of SEQ ID NO: 1;
(Pn2) a polynucleotide that encodes a protein having an activity of degrading inosinic acid and that has a base sequence consisting of the base sequence of (Pn1) above with at least one mutation selected from the group consisting of a deletion, a substitution, an insertion, and an addition of one or more bases:
(Pn3) a polynucleotide that encodes a protein having the activity of degrading inosinic acid and that has a base sequence having 90% or more identity to the base sequence of (Pn1) above;
(Pn4) a polynucleotide that encodes a protein having the activity of degrading inosinic acid and that has a base sequence complementary to a polynucleotide capable of hybridizing to the polynucleotide having the base sequence of (Pn1) above under a stringent condition:
(Pn5) a polynucleotide encoding a protein having an amino acid sequence of SEQ ID NO: 2;
(Pn6) a polynucleotide encoding a protein that has the activity of degrading inosinic acid and that has an amino acid sequence consisting of the amino acid sequence of SEQ ID NO: 2 with at least one mutation selected from the group consisting of a deletion, a substitution, an insertion, and an addition of one or more amino acids; and
(Pn7) a polynucleotide encoding a protein that has the activity of degrading inosinic acid and that has an amino acid sequence having 90% or more identity to an amino acid sequence of SEQ ID NO: 2.

8. The genetically modified fish according to claim 2,
wherein the fish is a red seabream, and
the red seabream comprises, as the inactivated nt5e gene, a mutant gene having a mutation in at least one exon selected from the group consisting of exon 1 and exon 6 of the normal nt5e gene.

9. (canceled)

10. (canceled)

11. The genetically modified fish according to claim 2,
wherein the fish is a tiger puffer, and
the normal nt5e gene of the tiger puffer is a gene that comprises a polynucleotide (Pt) below:
(Pt) any polynucleotide selected from the group consisting of polynucleotides (Pt1) to (Pt7):
(Pt1) a polynucleotide having a base sequence of SEQ ID NO: 3;
(Pt2) a polynucleotide that encodes a protein having an activity of degrading inosinic acid and that has a base sequence consisting of the base sequence of (Pt1) above with at least one mutation selected from the group consisting of a deletion, a substitution, an insertion, and an addition of one or more bases;
(Pt3) a polynucleotide that encodes a protein having the activity of degrading inosinic acid and that has a base sequence having 90% or more identity to the base sequence of (Pt1) above;
(Pt4) a polynucleotide that encodes a protein having the activity of degrading inosinic acid and that has a base sequence complementary to a polynucleotide capable of hybridizing to the polynucleotide having the base sequence of (Pt1) above under a stringent condition;
(Pt5) a polynucleotide encoding a protein having an amino acid sequence of SEQ ID NO: 4;
(Pt6) a polynucleotide encoding a protein that has the activity of degrading inosinic acid and that has an amino acid sequence consisting of the amino acid sequence of SEQ ID NO: 4 with at least one mutation selected from the group consisting of a deletion, a substitution, an insertion, and an addition of one or more amino acids; and
(Pt7) a polynucleotide encoding a protein that has the activity of degrading inosinic acid and that has an amino acid sequence having 90% or more identity to the amino acid sequence of SEQ ID NO: 4.

12. The genetically modified fish according to claim 2,
wherein the fish is a tiger puffer, and
the tiger puffer comprises, as the inactivated nt5e gene, a mutant gene having a mutation in exon 1 of the normal nt5e gene.

13. (canceled)

14. (canceled)

15. The genetically modified fish according to claim 2,
wherein the fish is a tilapia, and
the normal nt5e gene of the tilapia is a gene that comprises a polynucleotide (Po) below:
(Po) any polynucleotide selected from the group consisting of polynucleotides (Po1) to (Po7):
(Po1) a polynucleotide having a base sequence of SEQ ID NO: 5;
(Po2) a polynucleotide that encodes a protein having an activity of degrading inosinic acid and that has a base sequence consisting of the base sequence of (Po1) above with at least one mutation selected from the group consisting of a deletion, a substitution, an insertion, and an addition of one or more bases;
(Po3) a polynucleotide that encodes a protein having the activity of degrading inosinic acid and that has a base sequence having 90% or more identity to the base sequence of (Po1) above;
(Po4) a polynucleotide that encodes a protein having the activity of degrading inosinic acid and that has a base sequence complementary to a polynucleotide capable of hybridizing to the polynucleotide having the base sequence of (Po1) above under a stringent condition;
(Po5) a polynucleotide encoding a protein having an amino acid sequence of SEQ ID NO: 6;
(Po6) a polynucleotide encoding a protein that has the activity of degrading inosinic acid and that has an amino acid sequence consisting of the amino acid sequence of SEQ ID NO: 6 with at least one selected from the group consisting of a deletion, a substitution, an insertion, and an addition of one or more amino acids; and
(Po7) a polynucleotide encoding a protein that has the activity of degrading inosinic acid and that has an amino acid sequence having 90% or more identity to the amino acid sequence of SEQ ID NO: 6.

16. The genetically modified fish according to claim 2,
wherein the fish is a tilapia, and
the tilapia comprises, as the inactivated nt5e gene, a mutant gene having a mutation in exon 1 of the normal nt5e gene.

17. (canceled)

18. (canceled)

19. The genetically modified fish according to claim 2,
wherein the fish is a bastard halibut, and
the normal nt5e gene of the bastard halibut is a gene that comprises a polynucleotide (Pp) below:
(Pp) any polynucleotide selected from the group consisting of polynucleotides (Pp1) to (Pp7):
(Pp1) a polynucleotide having a base sequence of SEQ ID NO: 11;
(Pp2) a polynucleotide that encodes a protein having an activity of degrading inosinic acid and that has a base sequence consisting of the base sequence of (Pp1) above with at least one mutation selected from the group consisting of a deletion, a substitution, an insertion, and an addition of one or more bases;
(Pp3) a polynucleotide that encodes a protein having the activity of degrading inosinic acid and that has a base sequence having 90% or more identity to the base sequence of (Pp1) above;
(Pp4) a polynucleotide that encodes a protein having the activity of degrading inosinic acid and that has a base sequence complementary to a polynucleotide capable of hybridizing to the polynucleotide having the base sequence of (Pp1) above under a stringent condition;
(Pp5) a polynucleotide encoding a protein having an amino acid sequence of SEQ ID NO: 12;
(Pp6) a polynucleotide encoding a protein that has the activity of degrading inosinic acid and that has an amino acid sequence consisting of the amino acid sequence of SEQ ID NO: 12 with at least one mutation selected from the group consisting of a deletion, a substitution, an insertion, and an addition of one or more amino acids; and
(Pp7) a polynucleotide encoding a protein that has the activity of degrading inosinic acid and that has an amino acid sequence having 90% or more identity to the amino acid sequence of SEQ ID NO: 12.

20. The genetically modified fish according to claim 2,
wherein the fish is a bastard halibut, and
the bastard halibut comprises, as the inactivated nt5e gene, a mutant gene having a mutation in exon 4 of the normal nt5e gene.

21. (canceled)

22. (canceled)

23. The genetically modified fish according to claim 2,
wherein the fish is a whitespotted clarias, and
the normal nt5e gene of the whitespotted clarias is a gene that comprises a polynucleotide (Pq) below:
(Pq) any polynucleotide selected from the group consisting of polynucleotides (Pq1) to (Pq7):
(Pq1) a polynucleotide having a base sequence of SEQ ID NO: 25;
(Pq2) a polynucleotide that encodes a protein having an activity of degrading inosinic acid and that has a base sequence consisting of the base sequence of (Pq1) above with at least one mutation selected from the group consisting of a deletion a substitution an insertion, and an addition of one or more bases;
(Pq3) a polynucleotide that encodes a protein having the activity of degrading inosinic acid and that has a base sequence having 90% or more identity to the base sequence of (Pq1) above;
(Pq4) a polynucleotide that encodes a protein having the activity of degrading inosinic acid and that has a base sequence complementary to a polynucleotide capable of hybridizing to the polynucleotide having the base sequence of (Pq1) above under a stringent condition;
(Pq5) a polynucleotide encoding a protein having an amino acid sequence of SEQ ID NO: 26;
(Pq6) a polynucleotide encoding a protein that has the activity of degrading inosinic acid and that has an amino acid sequence consisting of the amino acid sequence of SEQ ID NO: 26 with at least one mutation selected from the group consisting of a deletion, a substitution, an insertion, and an addition of one or some amino acids; and
(Pq7) a polynucleotide encoding a protein that has the activity of degrading inosinic acid and that has an amino acid sequence having 90% or more identity to the amino acid sequence of SEQ ID NO: 26.

24. The genetically modified fish according to claim 2,
wherein the inactivated nt5e gene encodes a mutant NT5E protein with an activity of degrading inosinic acid that is reduced as compared to that of the normal nt5e gene.

25. The genetically modified fish according to claim 1,
wherein the fish is a fish selected from the group consisting of the family Tetraodontidae, the family Sparidae, the family Salmonidae, the family Cyprinidae, the family Serranidae, the family Paralichthyidae, the family Clariidae, and the family Cichlidae.

26. (canceled)

27. A fish production method comprising a step (a) below:
(a) mating the genetically modified fish according to claim 1 with another fish.

28. (canceled)

29. (canceled)

30. A method for screening an accelerated genetically modified ripened fish, comprising selecting, from a test sample of fish, a genetically modified test fish in which a function of an ecto-5′-nucleotidase (nt5e) gene is lost as an accelerated ripened fish.

31. (canceled)

32. (canceled)

33. (canceled)
